(19) United States
(12) Patent Application Publication
(10) Pub. No.: US 2010/0234379 A1
(43) Pub. Date: Sep. 16, 2010

(54) Dual modulation of endocannabinoid transport and fatty-acid amide hydrolase for treatment of excitotoxicity

(76) Inventors: Ben A. Bahr, Hampton, CT (US); Alexandros Makriyannis, Watertown, MA (US); David A. Karanian, Wethersfield, CT (US)

Correspondence Address: ALIX YALE & RISTAS LLP, 750 MAIN STREET, SUITE 1400, HARTFORD, CT, 06103, US

(21) Appl. No.: 11/494,852

(22) Filed: Jul. 28, 2006

Related U.S. Application Data
(60) Provisional application No. 60/703,368, filed on Jul. 28, 2005.

(51) Int. Cl. A61K 31/5377; A61K 31/10; A61K 31/255; A61K 31/12; A61K 31/27; A61K 31/4402; A61K 31/4245; A61K 31/428; A61K 31/167; A61K 31/16; A61K 31/454; A61P 25/00

(52) U.S. Cl. 514/236.5; 514/709; 514/711; 514/517; 514/678; 514/679; 514/479; 514/357; 514/352; 514/364; 514/373; 514/627; 514/326

(57) ABSTRACT

The endocannabinoid transporter and FAAH are sites of modulation that allow pharmacological enhancement of protective endocannabinergic signals. Selective inhibitors of the transporter and inhibitors of FAAH caused additive augmentation of endogenous signaling events mediated by the cannabinoid CB1 receptor. Disruption of such signals has been shown to prevent neuronal maintenance processes and increase vulnerability to brain damage. Here, blocking endocannabinoid inactivation enhanced cannabinergic activity and ameliorated cellular disturbances associated with excitotoxicity. Modulating the endocannabinoid system in this way also prevented excitotoxic behavioral abnormalities including memory impairment. Collectively, these results indicate that increasing endocannabinoid responses by inhibiting the endocannabinoid transported and/or the inhibiting FAAH leads to molecular, cellular, and functional protection against excitotoxic insults like stroke and traumatic brain injury.

### CROSS REFERENCE TO RELATED APPLICATIONS

This application is a regular utility application which claims the benefit of United States Provisional Application No. 60/703,368, filed Jul. 28, 2005, the contents of which are incorporated by reference in their entirety.

### STATEMENT REGARDING FEDERALLY SPONSORED RESEARCH OR DEVELOPMENT

This invention was made with Government support under U.S. Army Medical Research grant DAMD17-99-C9090, and NIH grants NIH1R3NS38404-1, DA07312, DA07215, and DA09158. The Government may have certain rights in the invention.

### FIELD OF THE INVENTION

The present invention relates generally to a method of preventing or treating excitotoxicity.

### SUMMARY

The classical cannabinoid Δ⁹-Tetrahydrocannabinol (Δ⁹-THC) is the major active constituent extracted from Cannabis sativa. The effects of such cannabinoids are due to an interaction with specific high-affinity receptors. Presently, two cannabinoid receptors have been characterized: CB1, a central receptor found in the mammalian brain and a number of other sites in peripheral tissues; and CB2, a peripheral receptor found principally in cells related to the immune system.

The effects of cannabinoids, including naturally occurring endocannabinoids, are affected by the direct activation of CB1 receptors as well as 1) a specific transporter system and 2) a fatty-acid amide hydrolase (FAAH). Together, these make up the endocannabinoid system. The transporter system and FAAH are part of a two-step process for inactivation of cannabinoid compounds.

Anandamide released by depolarized neurons is believed to be subject to rapid cellular uptake followed by enzymatic degradation. Indeed, rat brain neurons and astrocytes in primary culture avidly take up radioactively labeled anandamide through a mechanism that meets four key criteria of a carrier-mediated transport; temperature dependence, high affinity, substrate selectivity, and saturation. In that other lipids including polyunsaturated fatty acids and prostaglandin E₂ (PGE₂) enter cells by carrier-mediated transport, it is possible that anandamide uses a similar mechanism. This accumulation may result from the activity of a transmembrane carrier or transporter, which may thus participate in termination of the biological actions of anandamide. This carrier or anandamide transporter is believed to be involved in the inactivation of anandamide. Thus, anandamide released from neurons on depolarization may be rapidly transported back into the cells and subsequently hydrolyzed by an amidase thereby terminating its biological actions. Internalization of endocannabinoids is facilitated by the highly selective carrier-mediated transport system. Inhibitors of this transport system have been shown to enhance the effects of exogenous cannabinoid ligands. Thus, inhibitors of the endocannabinoid/anandamide transport system have the effect of indirectly stimulating the CB1 receptors by increasing the time that cannabinoids are available in vivo. It should be understood that the present disclosure encompasses inhibitors of endocannabinoid transport and/or anandamide transport and that the words transport, endocannabinoid transport and anandamide transport are used interchangeably in this application.

Certain analogs of anandamide are potent inhibitors of transport of anandamide across cell membranes. The transport inhibitor does not activate the cannabinoid receptors or inhibit anandamide hydrolysis per se but instead prevents anandamide reuptake thereby prolonging the level of the undegraded anandamide. The anandamide transport inhibitors suitable for use in accordance with the disclosed methods target the activity of the anandamide transporter.

Fatty acid amide hydrolase (FAAH) belongs to the amidase signature (AS) super family of serine hydrolases and is an intracellular membrane-bound enzyme that degrades and inactivates members of the endocannabinoid class of signaling lipids such as anandamide, N-arachidonoyl ethanolamine (AEA, anandamide) and other related compounds.

As such, FAAH activity is the second step of endocannabinoid inactivation. The hydrolase is distributed throughout the brain, and is thought to be the primary mediator for the hydrolysis of released endocannabinoids. As with transport inhibition, disrupting FAAH activity also enhances endocannabinoid signaling. Thus, inhibitors of FAAH will slow down the hydrolysis of endocannabinoids and thereby have the effect of indirectly stimulating the CB1 receptors by increasing the time cannabinoids are available in vivo. See Makriyannis et al U.S. Pat. Nos. 5,688,825 and 5,874,459, the disclosures of which are incorporated by reference in their entirety.

Thus, cannabinoid signals can be enhanced by increasing the time that cannabinoids are available to interact with or stimulate receptors by using inhibitors of the endocannabinoid and/or anandamide transport system and/or inhibitors of FAAH.

Glutamate, an amino acid and a prominent excitatory neurotransmitter, is involved in the normal activation of neurons to develop their essential role in the functional activity of the brain. However, high concentrations of glutamate, or neurotoxins acting at the same receptors, cause cell death through the excessive activation of these receptors. Excitotoxicity involves this process in which excessive activation of neuron glutamate receptors leads to death of the neurons. Excitotoxicicity and excitotoxic events are found in a number of diseases.

The inventors believe that modulating the endocannabinoid and/or anandamide transporter system and/or FAAH activity, and thereby endocannabinoid inactivation mechanisms, leads to a level of CB1 signaling that is sufficient to protect against excitotoxicity in vitro and in vivo. By modulating inactivation processes with inhibitors of the endocannabinoid/anandamide transporter and/or inhibitors of the FAAH, endogenous CB1 signaling was found to be enhanced. The selective transporter inhibitor and FAAH inhibitor were combined in order to produce additive modulation of endocannabinoid tone. The efficient dual modulation of the endocannabinoid system was evaluated for neuroprotection against i) molecular indicators of pathology in the excitotoxic hippocampal slice model, ii) pathogenic indicators in vivo following intrahippocampal injection of excitotoxin, and iii) functional deficits induced in the hippocampal lesion model.

In one embodiment a novel method for preventing or treating excitotoxicity in a subject comprising administering a therapeutically effective amount of a material selected from one of an anandamide transport inhibitor and a fatty-acid amide hydrolase inhibitor, or a physiologically acceptable salt thereof, to the subject is disclosed.

In another embodiment a novel method for preventing or treating excitotoxicity in a subject comprising administering a therapeutically effective amount of a material selected from a combination of an anandamide transport inhibitor and a fatty-acid amide hydrolase inhibitor, or a physiologically acceptable salt thereof, to the subject is disclosed.

Exemplary transporter inhibitors and their preparation for use in the novel methods are disclosed in U.S. Patent Publications 2003/0149082; 2003/0120094; 2005/0020679; and International Publication WO 99/64389, the contents of each of which are incorporated by reference in their entirety. Exemplary FAAH inhibitors and their preparation are for use in the novel methods are disclosed in U.S. Pat. Nos. 5,688,825; 5,874,459; 6,391,909; and 6,579,900, the contents of each of which are incorporated by reference herein in their entirety. Some exemplary endocannabinoid and/or anandamide transporter inhibitors and some exemplary FAAH inhibitors will also be described later in this application.

The compounds suitable for use with the disclosed method include any and all isomers and stereoisomers. In general, the compositions of the invention may be alternately formulated to comprise, consist of, or consist essentially of, any appropriate components herein disclosed. The compositions of the invention may additionally, or alternatively, be formulated so as to be devoid, or substantially free, of any components, materials, ingredients, adjuvants or species used in the prior art compositions or that are otherwise not necessary to the achievement of the function and/or objectives of the present invention.

### BRIEF DESCRIPTION OF THE DRAWINGS

Referring now to the drawings:

FIG. 1. CB₁ signaling by direct receptor activation (FIGS. 1A, 1B) and disruption of endocannabinoid inactivation (FIGS. 1C, 1D). Hippocampal slice cultures were treated with selective agents for 30 min in groups of 6-8 slices each, then rapidly homogenized in the presence of phosphatase inhibitors for parallel assessment of pFAK, pERK2, and actin on single immunoblots. A: Untreated slices were used to determine basal antigen levels (lane 1), while other slices were exposed to the CB₁ agonist R-methanandamide (A) in the absence (lane 2) or presence of the antagonist H (lane 3). B: Integrated optical densities for pERK2 (mean±SEM) were determined by image analysis (ANOVA: P=0.014). C: Treatment groups include control slices (lane 1) and slices treated with the FAAH inhibitor B (lane 2), the transporter inhibitor C (lane 3), the B/C combination (lane 4), or B/C with 20 μM F (lane 5). D: Image analysis data for pERK2 are shown (ANOVA: P=0.015). Asterisks represent significant post-hoc tests compared to control and H-treated slices. As used in the specification and the accompanying drawings, B/C indicates a combination of two compounds B and C.

FIG. 2. Excitotoxic protection through direct CB₁ activation (FIGS. 2A, 2B) and disruption of endocannabinoid inactivation (FIGS. 2C, 2D) in vitro. Hippocampal slice cultures were infused with an excitotoxic level of AMPA for 20 min, followed by a 10-min washout period. A subset of cultures then received either the CB₁ agonist A (10 μM) or the combination of FAAH inhibitor B and transport inhibitor C (0.5 μM and 50 μM, respectively). The slices were harvested 24 h post-insult in groups of 6-8 each, along with untreated sister cultures, and assessed by immunoblotting for calpain-mediated spectrin breakdown product BDP_{N} (A, C) and the postsynaptic marker GluR1 (B, D). Representative antigen staining is shown with a protein load control (con) from the same blots. Graphs contain integrated optical densities (mean±SEM) as determined by image analysis.

FIG. 3. Direct CB₁ activation protects against hippocampal excitotoxicity in vivo. Groups of adult rats received a 2.5-μl unilateral injection into the dorsal hippocampus, containing vehicle only (veh; n=10), 63 nmol AMPA (insult; n=19), or AMPA co-administered with 250 nmol of the CB₁ agonist A (n=12). At 4-7 days post-injection, brains were rapidly removed under ice-cold conditions containing protease inhibitors, and the ipsilateral dorsal hippocampus dissected for immunoblotting. Spectrin breakdown product BDP_{N}, postsynaptic marker GluR1, presynaptic marker synapsin II, and actin were assessed on single immunoblots (FIG. 3A). Mean integrated optical densities ±SEM are shown for BDP_{N} (FIG. 3B; ANOVA: P<0.0001) and GluR1 (FIG. 3C; P<0.001). Post-hoc tests compared to insult only data: *P<0.05, **P<0.001.

FIG. 4. Disruption of endocannabinoid inactivation protects against hippocampal excitotoxicity in vivo. Adult rats received a 2.5-μl unilateral injection into the dorsal hippocampus, containing vehicle only (group 1; n=13), a 63-nmol AMPA insult (group 2; n=20), the AMPA insult co-administered with 0.75 nmol B and 75 nmol C (group 3; n=12), AMPA co-administered with both the B/C combination and 43 nmol of the CB₁ antagonist G (group 4; n=5), or G alone (see lane 5; n=3). At 4-7 days post-injection, ipsilateral dorsal hippocampal tissue was rapidly dissected using ice-cold buffers with protease inhibitors. FIGS. 4A, 4B: Protection by the endocannabinoid transport and FAAH inhibitors is evident in blot samples from the five treatment groups (lanes 1-5, respectively), and the protection was blocked by G. G alone had no effect on the six antigens stained. Syn, synaptophysin.

Mean integrated optical densities ±SEM are shown for BDP_{N} (C; ANOVA: P<0.0001) and GluR1 (D; P<0.0001). Post-hoc tests compared to insult only data: *P<0.05, **P<0.001.

FIG. 5. Disruption of endocannabinoid inactivation promotes cell survival. Rats subjected to dorsal hippocampal injections as in FIG. 4 were sacrificed 7 days post-insult, and brains were rapidly fixed for histology or dissected for immunoblot analyses. FIG. 5A: Nissl-stained coronal sections from 9 animals confirmed lesion size and location as depicted by black circles (mm values are posterior to bregma). FIG. 5B: Ipsilateral dorsal hippocampus from rats injected with vehicle only (lane 1) or the 63-nmol AMPA insult (lane 2) were assessed for BDP_{N} and GluR1 along side the contralateral sample from the AMPA-treated animal (lane 3). Photomicrographs of the ipsilateral CA1 field are shown for animals injected with vehicle (FIG. 5C), AMPA (FIG. 5D), or AMPA co-administered with 0.75 nmol B and 75 nmol C (FIG. 5E). sp, stratum pyramidale; sr, stratum radiatum. Size bar: 30 μm.

FIG. 6 Disruption of endocannabinoid inactivation provides functional protection in the excitotoxic rat. FIG. 6A: In 5-8 rats per treatment group, a 2.5-μl unilateral injection was administered into the dorsal hippocampus, containing vehicle only, a 63-nmol AMPA insult, or the AMPA insult co-administered with 0.75 nmol B and 75 nmol C. At 4-7 days post-injection the animals were tested for turning behavior. Administration of B/C during the insult reduced AMPA-induced turning (left bars; ANOVA: p<0.01), while mean move time remained unchanged (right bars). FIG. 6B: Using a fear conditioning paradigm, different animals (n=13-14) were conditioned to 7 pairings of a tone that co-terminated with foot shock, then were subjected to the intrahippocampal injections. At 4-7 days post-injection, the animals were presented with tone in the absence of shock and assessed for freezing behavior. The AMPA insult alone caused memory impairment (left bars). Inhibiting endocannabinoid hydrolysis and transport with B/C, applied during the insult, reduced the memory impairment measured 4-7 days post-injection (ANOVA: p<0.0001). The baseline activity measured prior to the onset of the tone was unchanged across treatment groups (right bars). Post-hoc tests compared to insult alone: *P<0.05.

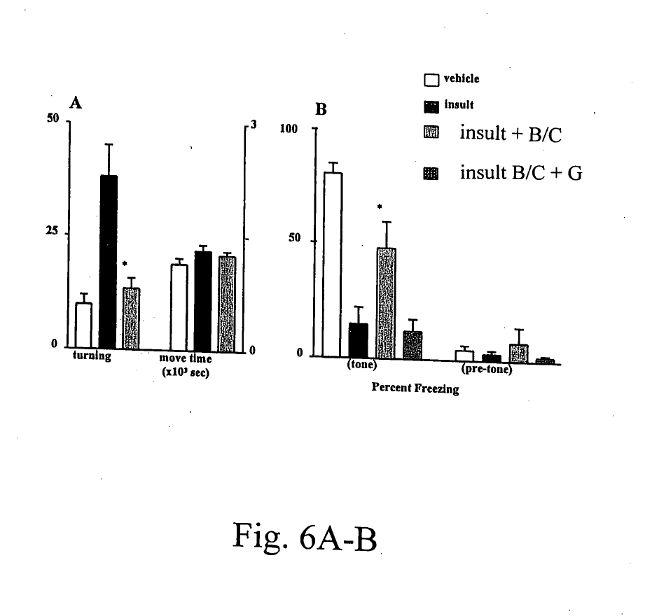

FIG. 7 Systemic administration of inhibitors of anandamide transport and FAAH reduces excitotoxin-induced seizure severity. Young rats were systemically administered control injections only (con) or, in order to induce seizures were injected with 10 mg/kg of the excitotoxin kainic acid. The excitotoxin-exposed animals were immediately given an intraperitoneal injection of vehicle (KA) or a drug combination consisting of the transport inhibitor D (0.5 mg/kg) and FAAH inhibitor E (1.0 mg/kg). Seizures were scored for 4 hours following excitotoxin exposure and are shown as mean±SEM. The D/E drug combination significantly reduced seizure severity throughout the 4 h rating period (*p<0.03 compared to KA alone).

FIG. 8 Systemic administration of inhibitors of anandamide transport and FAAH protects against excitotoxicity. Groups of young rats received intraperitoneal injections containing control only (con), kainic acid (KA), and KA followed by the D/E drug combination as described in FIG. 7. At 48 hours post-insult, brains were rapidly removed under ice-cold conditions containing protease inhibitors, and the hippocampus dissected for immunoblotting. The immunoblots stained with selective antibodies show that KA induced cytoskeletal break down (BDP_{N}), and caused a loss of synaptic markers (GluR1, Synapsin II). The inhibitors of anandamide transport and FAAH protect against the KA-induced neuronal damage.

FIG. 9 Systemic administration of inhibitors of anandamide transport and FAAH promotes cell survival. Rats were subjected to KA and drug injections as described in FIG. 7 and were sacrificed 48 hrs post-insult. The hippocampal tissue was rapidly fixed and sections were stained with Hematoxylin and Eosin to label neurons. KA-induced excitotoxicity caused a dramatic reduction in neuronal density as compared to control tissue. The drug combination of 0.5 mg/kg D and 1.0 mg/kg E shows clear evidence of neuroprotection. sp, stratum pyramidale; sr, stratum radiatum. Size bar: 30 μm.

FIG. 10 shows the structures of compounds A-H.

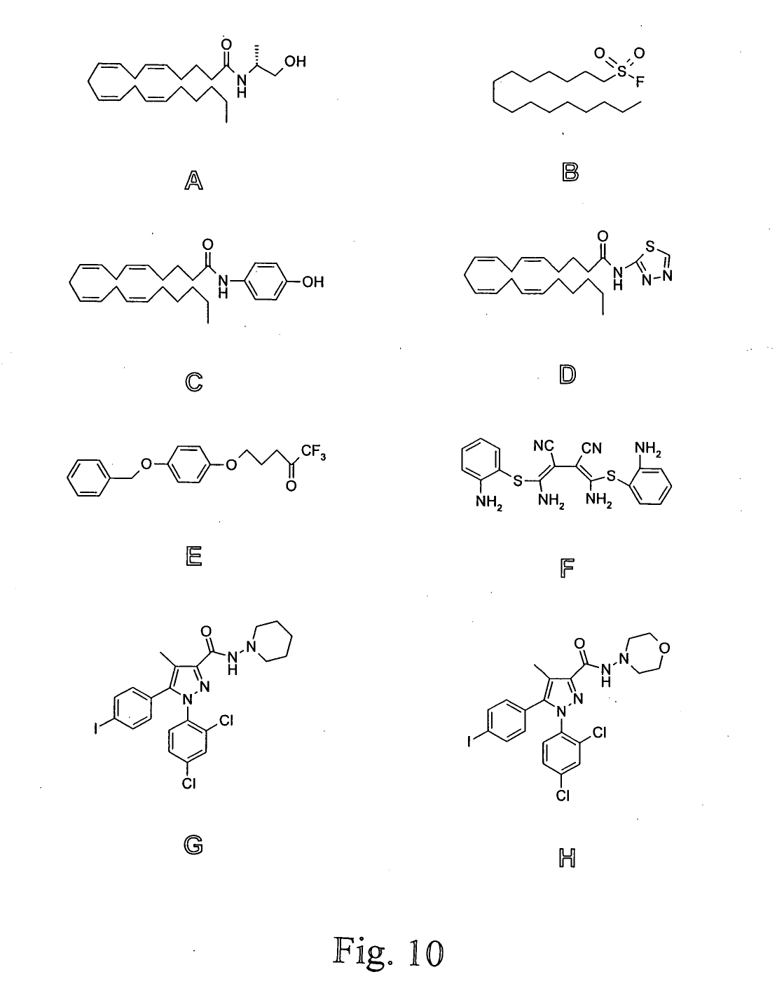

### DETAILED DESCRIPTION

This disclosure indicates that the endocannabinoid/anandamide transporter and FAAH are sites of modulation that allow pharmacological enhancement of protective endocannabinergic signals. Selective inhibitors of the transporter and inhibitors of the FAAH caused additive augmentation of endogenous signaling events mediated by the cannabinoid CB1 receptor. Such signals are believed to be necessary for neuronal maintenance processes and to decrease vulnerability to brain damage. The inventors have found that blocking endocannabinoid inactivation enhanced cannabinergic activity and ameliorated cellular disturbances associated with excitotoxicity. Modulating the endocannabinoid system in this way also prevented excitotoxic behavioral abnormalities including memory impairment. Collectively, these results provide a method of increasing endocannabinoid responses that leads to molecular, cellular, and functional protection against, and treatment for, excitotoxicity and excitotoxic events.

In one embodiment the dual modulation of the endocannabinoid system used a combination of the transport inhibitor C and the FAAH inhibitor B. C has been shown to enhance anandamide's action in vivo and in hippocampal slices. B also has been shown to increase the endocannabinoid's levels in neuroblastoma cells and to increase the modulatory action of low-level exogenous anandamide in hippocampus. Separately, the two drugs were found to trigger cannabinergic activation of FAK and ERK/MAPK pathways in the cultured hippocampal slice model. In combination they caused surprisingly pronounced FAK and ERK responses similar to those triggered by a CB1 direct agonist, and the ERK activation was selectively blocked by a MEK inhibitor. The ERK pathway is of particular interest since it promotes synaptic maintenance and cell survival, thus, it may be related to the excitotoxic protection elicited by endocannabinoids. Activation events elicited by B/C were also blocked by the CB1 antagonist H. As used in the specification and the accompanying drawings, B/C indicates a combination of compounds B and C. These data indicate that disruption of two distinct mechanisms of endocannabinoid inactivation—transport and hydrolysis—causes potentiation of endocannabinoid tone.

The hippocampus is abundant in cannabinoid CB1 receptors, which are found in many brain regions. A second type of cannabinoid receptor, the CB2 class, is only present in the periphery. The relationship between CB1 receptors and pro-survival FAK and ERK signaling appears to support the maintenance of hippocampal neurons and may explain the protective properties linked to the CB1 receptors. In addition, the CB1 receptors are involved in other repair systems including brain-derived neurotrophic factor that is dependent on the ERK/MAPK pathway, and phosphatidylinositol 3-kinase (PI3K) that makes up a potential FAK/PI3K/ERK repair pathway. It has also been suggested that direct activation of CB1 receptors by CB1 agonists may lead to the inhibition of voltage-sensitive calcium channels, perhaps contributing to reductions in excitotoxic progression. However, the present method “indirectly” activates the CB1 receptors by increasing endogenous signaling thereby allowing the body's own endocannabinoids to have a longer half-life (before being transported back into cells and/or being degraded by FAAH). The longer endocannabinoid half life allows greater protection when endocannabinoids are released in response to injury. The present method also lessens potential psychoactive problems stemming from chronic treatment with CB₁ agonists.

Levels of endocannabinoids are elevated after neuronal injury, indicating a potential compensatory response comprised of possibly several CB1-linked signaling events involved in cellular repair. The present document suggests that such compensatory signaling can be positively modulated through the inhibition of endocannabinoid transport and hydrolysis with the B/C drug combination. In vitro and in vivo models were used here to show that dual modulation of the endocannabinoid system protect against cellular and functional consequences ascribed to excitotoxic events, such as stroke and traumatic brain injury.

Cytoskeletal damage was assessed by measuring calpain-mediated spectrin breakdown product BDP_{N}, a sensitive precursor to neuronal pathology in animal models of excitotoxic insults and human brain injury. B/C reduced the excitotoxic damage induced in the hippocampus. The level of cytoskeletal protection produced by the drug combination was similar if not more robust than that elicited by the stable CB1 agonist A. Correspondingly, the B/C combination that promotes cytoskeletal protection also was found to promote cell survival.

Disrupting endocannabinoid inactivation with B/C also provided synaptic protection. Synaptic and dendritic compromise is often found associated with calpain-induced cytoskeletal damage. As shown here, AMPA-induced excitotoxic progression caused pre- and postsynaptic decline in the hippocampal slice model. In vivo, the pronounced level of synaptic decline evident in dorsal hippocampal tissue samples indicates global synaptopathogenesis produced by the excitotoxic insult. As with cytoskeletal protection, indirect modulation of the endocannabinoid system with B/C produced the same or better synaptic protection as compared to treatment with CB1 agonist. The protection of synaptic integrity would provide an added beneficial effect since the activity of glutamatergic synapses in hippocampus is vital for neuronal maintenance. Thus the neuroprotective effects may have extended beyond the site at which B/C was injected. In addition, the modulators of the endocannabinoid system preserved basal levels of activated FAK and ERK, two kinases involved in synaptic maintenance signaling and whose levels are dramatically reduced in the in vivo model of excitotoxicity.

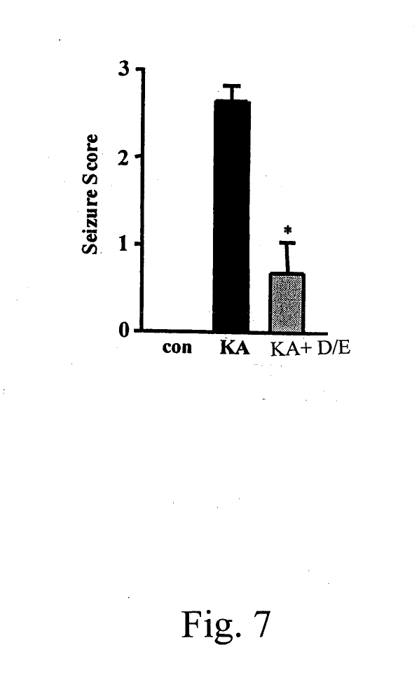

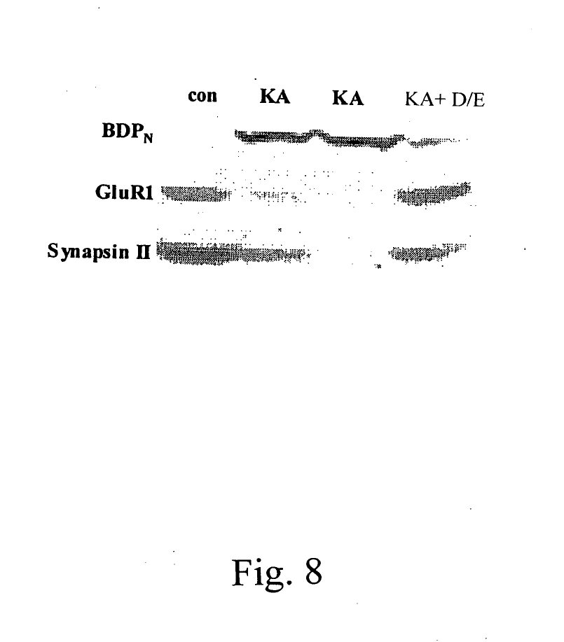

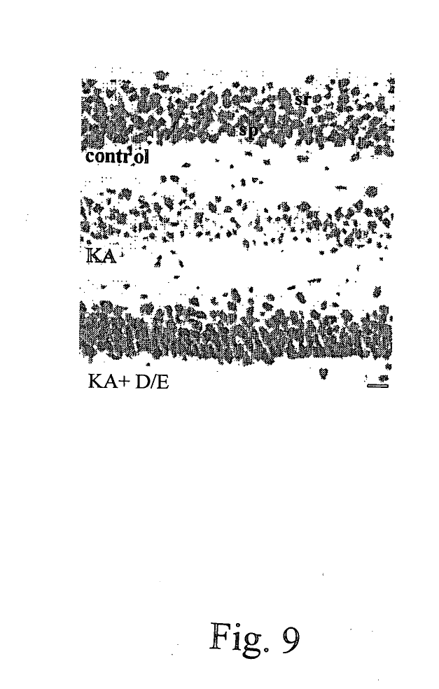

In addition to the cytoskeletal, synaptic, and cellular protection, two behavioral correlates of excitotoxic brain damage were also reduced by the B/C drug combination, implicating endocannabinoid modulation in the protection of normal brain function. The selective disturbance of perseverative turning in the unilateral excitotoxic rat model was prevented when B/C was co-administered with the insult. The excitotoxic animals also exhibited memory impairment as indicated by the lack of recall of pre-insult fear conditioning. The memory dysfunction was significantly prevented by the dual blockage of endocannabinoid inactivation mechanisms. Of particular interest is the corresponding preservation of the glutamate receptor subunit GluR1, synaptic vesicle markers, and basal levels of active ERK. Note that GluR1, the synaptic vesicle protein synaptotagmin IV, and the ERK/MAPK pathway within hippocampal neurons have been shown to play important roles in memory functions including fear conditioning. The results described here show that indirect enhancement of endocannabinoid responses protects against excitotoxic hippocampal damage and preserves mechanisms necessary for memory encoding. It should be noted that the data shown in FIGS. 7-9 involves systemic administration of the identified compounds as opposed to directly injecting the compounds into the brain.

Blocking endocannabinoid inactivation with the drug combination of B (an FAAH inhibitor) and C (a transporter inhibitor) protects against excitotoxicity both in vitro and in vivo. Such enhancement of cannabinergic responses provides a method with which to reduce potential problems stemming from chronic treatment with CB1 agonists. The results indicate that administration of compounds that inhibit the transporter and/or inhibit the FAAH result in an indirect enhancement of neuroprotective endocannabinoid signaling. This represents a novel method for dual modulation of the endocannabinoid system for therapeutic purposes such as to prevent or treat excitotoxicity and diseases in which excitotoxic events are found. Diseases in which excitotoxic events are found include stroke, brain injury, brain trauma, epilepsy, hypoxia, ischemia, toxin exposure, tumor growth and excitotoxicity linked to dementia such as in Alzheimer's Disease.

As used herein a “therapeutically effective amount” of a compound, is the quantity of a compound which, when administered to an individual or animal, results in a sufficiently high level of that compound in the individual or animal to cause a physiological response. The inventive compounds described herein and incorporated by reference, and physiologically acceptable salts thereof, have pharmacological properties when administered in therapeutically effective amounts for providing a physiological response useful to prevent or treat excitotoxicity

Typically, a “therapeutically effective amount” of an inventive compound is believed to range from about 5 mg/day to about 1,000 mg/day.

As used herein, an “individual” refers to a human. An “animal” refers to, for example, veterinary animals, such as dogs, cats, horses and the like, and farm animals, such as cows, pigs and the like.

As used herein, “purified” or “substantially pure” refers to the process(es) whereby an end product is purified to a desired degree for a particular purpose. A person of ordinary skill in the art would know to what degree of purity is required for a particular purpose and method to achieve that purity without undue effort. The purified compound may be used in any disclosed embodiment. The exemplary compounds should be understood to include all stereoisomers (geometric isomers, diastereomers and enantiomers).

The compounds suitable for use in accordance with the disclosed methods can be administered by a variety of known methods, including, for example, orally, rectally, or by parenteral routes (e.g., intramuscular, intravenous, subcutaneous, nasal or topical). The form in which the compounds are administered will be determined by the route of administration. Such forms include, but are not limited to, capsular and tablet formulations (for oral and rectal administration), liquid formulations (for oral, intravenous, intramuscular, subcutaneous, ocular, intranasal, inhalation-based and transdermal administration) and slow releasing microcarriers (for rectal, intramuscular or intravenous administration). The formulations can also contain a physiologically acceptable vehicle and optional adjuvants, excipients, stabilizers, flavorings, colorants, and preservatives. Suitable physiologically acceptable vehicles include, but are not limited to, saline, sterile water, Ringer's solution and isotonic sodium chloride solutions. The specific dosage level of active ingredient will depend upon a number of factors, including, for example, biological activity of the particular preparation, age, body weight, sex and general health of the individual being treated.

Some exemplary compounds that may be purified and used in accordance with the disclosed methods include any of the following structure types and/or physiologically acceptable salts thereof:

In one embodiment, compounds that can be used to inhibit endocannabinoid and/or anandamide transport have a structure represented by Structural Formula I

X—Y—Z. (I)

The tail portion X is a fatty acid chain remnant, or an aliphatic hydrocarbon as defined later, or a biphenyl group with an alkyl chain.

Generally speaking, anandamide transport inhibitors may include, for example, amide, reverse amide or carbonyl amine, urea, carbamate and ester analogs of anandamide having the three pharmacophores of the Structural Formula I wherein the tail portion X is a fatty acid hydrophobic carbon chain having one or more nonconjugated cis double bonds in the middle portion of the aliphatic hydrocarbon chain or a biphenyl group having an alkyl or branched alkyl distal moiety of about 1 to about 10 carbon atoms. The biphenyl group may be substituted with 1-6 substituents including OH, CH₃, halogen, SCH₃, NH₂, NHCOR, SO₂NHR, NO₂. The fatty acid chain may contain four to thirty carbon atoms but preferably the chain length is about 10 to 28 carbon atoms and more preferably contains from about 17 to about 22 carbon atoms. The aliphatic hydrocarbon chain may terminate with an aryl or alkyl aryl group. By contrast, analogs with fully saturated chains or with a trans or terminal double bond fail to compete successfully with [³H]anandamide for transport and thus are ineffective as inhibitors. The central pharmacophore Y is selected from the groups as set forth below. However, compounds containing a free carboxylic acid, carboxyethyl and carboxymethyl groups, or a primary alcohol are inactive.

As used herein, “aliphatic hydrocarbon” includes, unless otherwise stated, one or more polyalkylene groups connected by one or more cis-alkenyl linkages such that the total number of methylene carbon atoms is within the ranges set forth herein. The structure of some preferred tail portions have the formula II

CR′₃—(CR₂)ₐ-(cis-CH═CHCR₂)_{b}—(CR₂)_{c}— (II)

wherein R is selected from the groups consisting of hydrogen and lower alkyl groups, and R′ may be selected from hydrogen, lower alkyl groups as well as phenyl and biphenyl groups that are unsubstituted or substituted with a member selected from the group consisting of hydroxyl, halogen, —NO₂, —NH₂, —SCH₃, —CH₃ and —OCH₃, where “a” and “c” are each independently selected from 0 and an integer from 1 through 10 and b is an integer from 1 through 6. Specific examples include structures where X is CH₃—(CH₂)₄-(cis-CH═CHCH₂—)₄—(CH₂)₂—, CH₃—(CH₂)₄-(cis-CH═CHCH₂)₃—(CH₂)₅, —CH₃—(CH₂)₆-(cis-CH═CHCH₂)₂— (CH₂)₆—, CH₃— (CH₂)₆-(cis-CH═CHCH₂)₂— (CH₂)₅—CH₃—(CH₂)₇-cis CH═CH—(CH₂)₉, CH₃—(CH₂)₇-cis-CH═CH—(CH₂)₇— and CH₃—(CH₂)₄—(CH═CHCH₂)₄—CH₂—C(CH₃)₂—. A lower alkyl group is a straight or branched chain alkyl group having 1 to 5 carbon atoms, unless otherwise stated.

The central portion Y is a member selected from the group consisting of —NH—C(O)—, —NH—, —NH—C(O)—NH—, —NH—C(O)—O—, —O—C(O)—NH—, —C(O)—C(O)—NH, —NH—C(O)—C(O)—, —O—C(O)—O—, —C(O)—NH, —O—C(O)—, —C(O)—O—, —O—, —S— and —H. It should be noted that the X and Z portions may be connected to the Y portion at either of the Y portion connecting atoms. Thus, for example, the Y portion —C(O)—NH— will lead to analogs X—C(O)—NH—Z and Z—C(O)—NH—X.

The head portion Z is selected from the group consisting of hydrogen, aryl, substituted aryl, alkyl, hydroxy alkyl, alkyl aryl, hydroxy aryl, halogen substituted alkyl aryl, heterocyclics, hydroxy heterocyclic, cyclic glycerols and substituted cyclic glycerols, COCF₃, C(O)-alcohol, —(CH₂)ₘ—(C(CH₃)₂)ₚ—(CH₂)ₙ-T₂-T₃, —(CH₂)ₘ—(CH(CH₃))_{q}—(CH₂)ₙ-T₂-T₃ (where m and n are each independently selected from 0 to 6 integer, p and q are each independently 0 or 1, T₂ is optionally present and comprises aryl, a cyclic ring, a bicyclic ring, a tricyclic ring, a heterocyclic ring, a heterobicyclic ring, a heterotricyclic ring, a heteroaromatic ring, 1- or 2-glycerol, 1- or 2-cyclic glycerol, alkyl, alkenyl, alkynyl, T₃ comprises H, OH, SH, halogen, C(halogen)₃, CH(halogen)₂, O-alkyl, N₃, CN, NCS, NH₂, alkylamino, dialkylamino or a substituent group as defined later).

For this embodiment, an “aryl” group is a carbocyclic aromatic ring system such as phenyl, biphenyl 1-naphthyl or 2-naphthyl.

For this embodiment, “cyclic glycerols” include members selected from the group consisting of

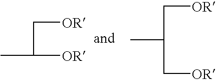

wherein R′ is a member selected form the group consisting of hydrogen, lower alkyl, aryl and substituted aryl radicals.

In some variations of endocannabinoid/anandamide transport inhibitors, the following provisos may apply.

When Y is —C(O)—N(H)— and X is the tail remnant of arachidonyl acid, Z excludes 4-hydroxyphenyl.

When Y is —O—C(O)—NH— and X is the tail remnant of arachidonyl acid, Z excludes ethyl, iso-propyl and propyl.

When Y is —NH—C(O)—NH— and X is the tail remnant of arachidonyl acid, Z excludes methyl, iso-propyl, propyl, iso-butyl, CH₂CH₂F, CH₂CH₂OH, and CH₂CH₂OCH₃.

When Y is —NH—C(O)—O— and X is the tail remnant of arachidonyl acid, Z excludes ethyl, iso-propyl and CH₂CH₂F.

When Y is —NH—C(S)—NH— and X is the tail remnant of arachidonyl acid, Z excludes 4-methyl-2-methoxy-phenol, and 4-methyl-2-chloro-phenol.

In another embodiment, some exemplary compounds that can be used to inhibit FAAH have a structure represented by Structural Formula III

R—X—Y (III)

and physiologically acceptable salts thereof.

R is selected from the group consisting of an alkyl group, a substituted alkyl group, an aryl group, a substituted aryl group, a heteroaryl group, a substituted heteroaryl group, a heterocyclic group and a substituted heterocyclic group.

X is a straight chain hydrocarbyl group or a substituted straight chain hydrocarbyl group containing from about 4 to about 18 carbon atoms if R is an aryl group, a substituted aryl group, a heteroaryl group, a substituted heteroaryl group, a heterocyclic group or a substituted heterocyclic group.

X is a hydrocarbyl group or a substituted hydrocarbyl group containing from about 10 to about 24 carbon atoms if R is a methyl group.

Y is a moiety capable of irreversibly binding with a nucleophilic group at the active site of an amidase enzyme.

“Y” in Structural Formula III is a moiety capable of irreversibly binding with a nucleophilic group at the active site of an amidase enzyme. Thus, Y is capable of forming a stable covalent bond with the nucleophilic group at the active site of an amidase enzyme. Suitable structures for Y therefore do not encompass moieties, such as trifluoromethyl ketones, which are capable of acting as a transition state analog of an amidase enzyme and which bind reversibly to these enzymes. As used herein, an “amidase” is an enzyme involved in the hydrolysis of an amide bond.

A nucleophilic group at the active site of an amidase enzyme is a heteroatom-containing functional group on the side chain of an amino acid found at the enzyme active site and includes the hydroxyl group of serine or threonine, the thiol group of cysteine, the phenol group of tyrosine and the amino group of lysine, ornithine or arginine or the imidazole group of histidine.

Examples of suitable structures for Y include:

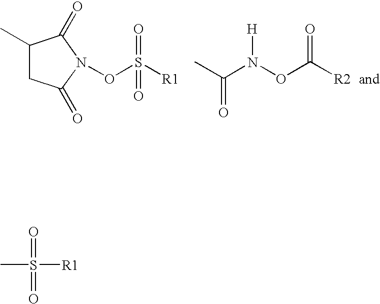

R1 is selected from the group consisting of —F and —O(C1 to C4 straight or branched chain alkyl group). R2 is a C1 to C4 straight or branched chain alkyl group.

As used herein, “a straight chain hydrocarbyl group” includes a polyalkylene, i.e., —(CH₂)ₙ—. “n” is a positive integer from about 10 to about 24, when R is methyl, and from about 4 to about 18, when R is aryl, substituted aryl, heteroaryl, substituted heteroaryl, heterocyclic or substituted heterocyclic. A straight chain hydrocarbyl group also includes two or more polyalkylene groups connected by one or more ether, thioether ether, cis-alkenyl, trans-alkenyl or alkynyl linkage such that the total number of methylene carbon atoms is from about 10 to about 24 when R is methyl and from about 4 to 18 when R is aryl, substituted aryl, heteroaryl, substituted heteroaryl, heterocyclic or substituted heterocyclic. Examples include —(CH₂)ₘ-0-(CH₂)ₒ—, —(CH₂)ₘ—S—(CH₂)ₒ—, —(CH₂)ₘ—CH═CH—(CH₂)ₒ—, —(CH₂)ₘ—C≡C—(CH₂)ₒ—, wherein m and o are each a positive integer such that the sum of m and o is equal to n. Some specific examples include where X is —(CH₂)₄—(cis-CH═CHCH₂—)₄—CH₂CH₂—, —(CH₂)₄-(cis-CH═CHCH₂)₃—(CH₂)₅— and where R—X— is a docosatetraenyl or a homo-γ-linolenyl moiety.

In one aspect, R in the compound being administered to inhibit anandamide amidase/FAAH is methyl and Y is sulfonyl fluoride or a C1 to C4 straight or branched chain sulfonyl ester. Preferably, Y is a sulfonyl fluoride. Specific examples of sulfonyl fluorides and sulfonyl esters include where R—X— is archidyl, Δ⁸, Δ¹¹, Δ¹⁴-eicosatrienyl, docosatetraenyl, homo-γ-linolenyl and CH₃—(CH₂)ₙ—, wherein n is 10 (lauryl), 11, 12 (myristyl), 13, 14 (palmityl), 15 or 16 (stearyl).

In another embodiment, compounds that can be used to inhibit FAAH have a structure represented by Structural Formula (IV):

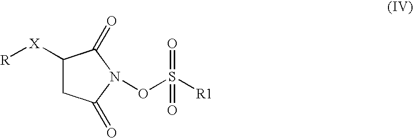

and physiologically acceptable salts thereof.
R1 is —F or (C1 to C4 alkyl)O—. R and X are as defined above for Structural Formula (III).

In another embodiment, compounds that can be used to inhibit FAAH have a structure represented by Structural Formula (V):

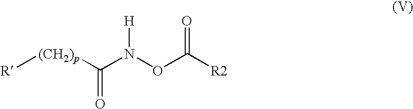

and physiologically acceptable salts thereof.

R′ is selected from the group consisting of an aryl group, a substituted aryl group, a heteroaryl group, a substituted heteroaryl group, a heterocyclic group and a substituted heterocyclic group.

R2 is a C1 to C4 straight or branched chain alkyl group and p is an integer from about 6 to about 18. In another aspect, p is an integer from about 10 to about 18.

The synthesis of the anandamide amidase/FAAH inhibitor compounds is described in the following US patents, U.S. Pat. No. 6,391,909, U.S. Pat. No. 5,688,825 and U.S. Pat. No. 5,874,459, the contents of which are incorporated in their entirety.

In another embodiment, compounds that can be used to inhibit FAAH are represented by the general formula VI and physiologically acceptable salts thereof. In the general formula VI, Y represents the inhibition subunit pharmacophore, and R—X represents the binding subunit pharmacophore. The exemplary compounds should be understood to include all stereoisomers (geometric isomers, diastereomers and enantiomers).

R—X—Y (VI)

wherein:

Y is selected from the following structures:

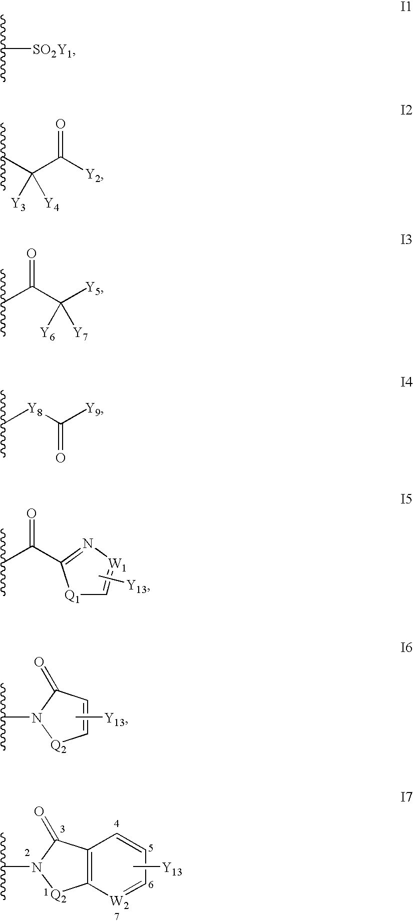

Y₁ is selected from —F, —Cl, —O-alkyl, —O-cycloalkyl, —O-heterocyclic, —O-aryl, —O-heteroaryl and —O-adamantlyl.

Y₂ is selected from —H, —OH, —NH₂, —OMe, —OEt, —CF₃, —C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄, -aryl-Y₁₄, -heteroaryl-Y₁₄, -cycloalkyl-Y₁₄, -heterocyclic-Y₁₄ and -adamantyl-Y₁₄.

Y₃ and Y₄ are each independently selected from —F, —Cl and —OH or Y₃ and Y₄ together form an oxo group, that is Y3 and Y4 together with the common carbon atom form the structure >C=0.

Y₅ is selected from —F, —CONH₂, —SO₂NH₂, —COOH, —COOMe, —COOEt, —CF₃, C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄, -aryl-Y₁₄, -heteroaryl-Y₁₄, -cycloalkyl-Y₁₄, -adamantyl-Y₁₄ and -heterocyclic-Y₁₄.

Y₆ and Y₇ are each independently selected from —F, —Cl and —OH.

Y₈ is selected from >NH and —O—.

Y_{g} is selected from —OY₁₀ and —N(Y₁₁)Y₁₂.

Y₁₀ is selected from alkyl, aryl, heteroaryl, cycloalkyl, adamantyl, heterocyclic, —C₁₋₅-alkyl-Y₁₄, -aryl-Y₁₄, -heteroaryl-Y₁₄, -cycloalkyl-Y₁₄, -adamantyl-Y₁₄ and -heterocyclic-Y₁₄.

Y₁₁ is —H.

Y₁₂ is selected from alkyl, aryl, heteroaryl, cycloalkyl, adamantyl, heterocyclic, —C₁₋₅-alkyl-Y₁₄, —C₁₋₅-alkyl-aryl, —C₁₋₅-alkyl-heteroaryl, -aryl-Y₁₄, -heteroaryl-Y₁₄, -cycloalkyl-Y₁₄, -adamantyl-Y₁₄ and -heterocyclic-Y₁₄, or Y₁₁ and Y₁₂ together comprise part of a 5 or 6 membered saturated heterocyclic ring containing up to one additional heteroatom selected from N, O and S.

Y₁₃ is selected from —H, —OH, —SH, —NH₂, —CN, —N₃, —NCS, —NCO, —CONH₂, —SO₂NH₂, —COOH, —COOMe, —COOEt, —NO₂, —CF₃, —SO₃H, —P(O)(OH)₂, —C≡CH, —CH₂C≡CH, —CH═CH₂, fluoroalkyl, —C₁₋₆-alkyl, aryl, heteroaryl, cycloalkyl, adamantyl, heterocyclic, —C₁₋₆-alkyl-Y₁₄, -aryl-Y₁₄, -heteroaryl-Y₁₄, -cycloalkyl-Y₁₄, -adamantyl-Y₁₄ and -heterocyclic-Y₁₀.

Y₁₄ is selected from —OH, —SH, —NH₂, —CN, —N₃, —NCS, —NCO, —CONH₂, —SO₂NH₂, —COOH, —COOMe, —COOEt, —NO₂, —CF₃, —SO₃H, —P(O)(OH)₂, —CH₂—C≡CH and —CH═CH₂.

W₁ is selected from CH and N if Y₁₃ is not bonded to W₁, or W₁ is C if Y₁₃ is bonded to W₁.

W₂ is selected from CH and N if W₂ is not bonded to Y₁₃, or W₂ is C if W₂ is bonded to Y₁₃. If W₂ is N then it can occupy any position selected from 4, 5, 6 and 7 in 17.

Q₁ is selected from >CH₂, >0, >S and >NH if Q₁ is not bonded to Y₁₃, or Q₁ is selected from >CH and >N if Q₁ is bonded to Y₁₃.
Q₂ is selected from >SO₂, >C(O) and >S(O).
X is selected from —(CH₂)ₙ— and —(CH₂)ⱼ-A-(CH₂)ₖ—.
   A is selected from —CH═CH—, C═O, O, S and NH.
n is an integer from 0 to about 15.
j is an integer from 0 to about 10.
k is an integer from 0 to about 10.

R is selected from the following structures:

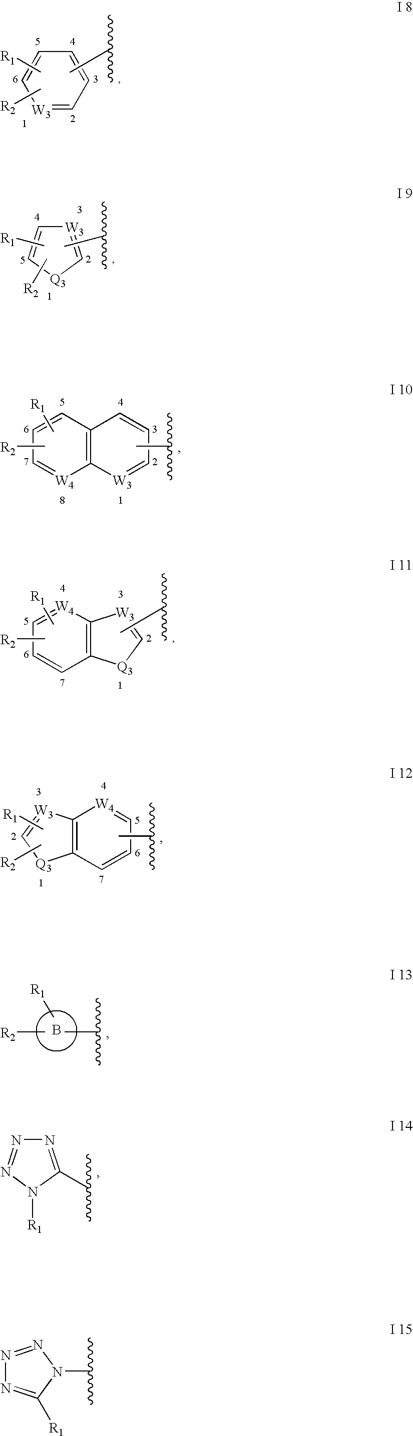

### Wherein:

W₃ is selected from CH and N if W₃ is not bonded to X or R₁ or R₂, or W₃ is C if W₃ is bonded to X or R₁ or R₂. If W₃ is N then it can occupy any position selected from 1, 2, 3, 4, 5 and 6 in I 8; 2, 3, 4 and 5 in I 9; 1, 2, 3 and 4 in I 10; 2 and 3 in I 11, I 12.

W₄ is selected from CH, N if W₄ is not bonded to X or R₁ or R₂, or W₄ is C if W₄ is bonded to X or R₁ or R₂. If W₄ is N then it can occupy any position selected from 5, 6, 7 and 8 in I 10; 4, 5, 6 and 7 in I 11, I 12.

Q₃ is selected from CH₂, O, S and NH if Q₃ is not bonded to X or R₁ or R₂, or Q₃ is selected from CH and N if Q₃ is bonded to X or R₁ or R₂.

B is an adamantyl or a heteroadamantyl ring.

R₁ and R₂ are each independently selected from —H, —F, —Cl, —Br, —I, —OH, —SH, —NH₂, —CN, —N₃, —NCS, —NCO, —CONH₂, —SO₂NH₂, —COON, —NO₂, —CHO, —CF₃, —SO₃H, —SO₂Cl, —SO₂F, —O—P(O)(OH)₂, —O—P(O)(O-alkyl)₂, —O—P(O)(OH)(O-alkyl), —P(O)(O-alkyl)₂, —P(O)(OH)(O-alkyl), —Sn(alkyl)₃, —Si(alkyl)₃, —CH₂—C≡CH, —CH═CH₂, -alkyl-R₃, -cycloalkyl-R₃, -heterocyclic-R₃, -aryl-R₃, -heteroaryl-R₃, -alkyl-cycloalkyl-R₃, -alkyl-heterocyclic-R₃, -alkyl-aryl-R₃, -alkyl-heteroaryl-R₃, —Z-alkyl-R₃, —Z-cycloalkyl-R₃, —Z-heterocyclic-R₃, —Z-aryl-R₃, —Z-heteroaryl-R₃, —Z-alkyl-cycloalkyl-R₃, —Z-alkyl-heterocyclic-R₃, —Z-alkyl-aryl-R₃, —Z-alkyl-heteroaryl-R₃, -aryl-Z-alkyl-R₃, -aryl-Z-cycloalkyl-R₃, -aryl-Z-heterocyclic-R₃, -aryl-Z-aryl-R₃, -aryl-Z-heteroaryl-R₃, -aryl-Z-alkyl-cycloalkyl-R₃, -aryl-Z-alkyl-heterocyclic-R₃, -aryl-Z-alkyl-aryl-R₃, -aryl-Z-alkyl-heteroaryl-R₃, —CH(alkyl-R₃)₂, —C(alkyl-R₃)₃, —N(alkyl-R₃)₂, —C(O)N(alkyl-R₃)₂ and —SO₂N(alkyl-R₃)₂.

Z is selected from —O, —S, —NH, —C(O), —C(O)O, —OC(O), —C(O)NH, —NHC(O), —SO, —SO₂, —SO₂NH, —NHSO₂, —SO₂O and —OSO₂.

R₃ is selected from —H, —F, —Cl, —Br, —I, -Me, -Et, —OH, —OAc, —SH, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —C≡CH, —CH₂—C≡CH and —CH═CH₂.

The following provisos may apply to some of the disclosed embodiments of formula VI.

If Y is —SO₂—Y₁ (I 1) where Y₁ is F or O-alkyl and X is —(CH₂)ₙ— where n=4-15 or —(CH₂)ⱼ-A-(CH₂)ₖ—, where A is selected from O, S, —CH═CH— and —C≡C—, j and k are each a positive integer such that the sum of j and k is equal to 4-15 and R is I 8, I 9, I 10, I 11 or I 12 where R₁ is H; then R₂ can not be H, F, Cl, Br, I, NO₂, CF₃, CN, CHO, aryl-R₃, heteroaryl-R₃, O-alkyl-R₃, O-aryl-R₃, C(O)—O-alkyl-R₃, C(O)-alkyl-R₃, C(O)NH-alkyl-R₃, C(O)N(alkyl-R₃)₂ or S-alkyl-R₃, where R₃═H.

If Y is I 3 where Y₅ is F, Y₆ is F, Y₇ is F and X is —(CH₂)ₙ— where n=5-7; then R can not be phenyl, 2-hexyl-phenyl, 3-hexyl-phenyl, 4-heptyl-phenyl or 2-octyl-phenyl.

If Y is I 3 where Y₅ is F, Y₆ is F, Y₇ is F and X is —(CH₂)ₙ— where n=3; then R can not be 2-butyl-naphthyl.

If Y is I 4 where Y₈ is NH and Y₉ is OY₁₀ where Y₁₀ is alkyl, phenyl, pyridyl or C₁₋₅-alkyl-Y₁₄ where Y₁₄═NH₂ or NO₂ and X is —(CH₂)ₙ— where n=0-3; then R can not be naphthyl, indolyl or I 8 where W₁ is CH and R₁ and R₂ are each selected from O—C₁₋₁₆-alkyl, O—C₁₋₁₆-alkyl-phenyl, O—C₁₋₁₆-alkyl-pyridyl, phenyl, O-phenyl, O-pyridyl or C(O)NH—C₁₋₁₆-alkyl.

If Y is I 5 where W₁ is CH or N, Q₁ is O or S, Y₁₃ is H, C₁₋₆-alkyl, aryl or heteroaryl and X is —(CH₂)ₙ— where n=3-9 or X is —(CH₂)ⱼ-A-(CH₂)ₖ— where A is O, S or NH and the sum of j and k is equal to 2-8; then R cannot be aryl.

If Y is I 5 where W₁ is N, Q₁ is O or S, Y₁₃ is selected from phenyl, 2-pyridyl, 3-pyridyl, 4-pyridyl and 2-furyl and X is —(CH₂)ₙ— where n=5-8; then R can not be I 8 where W₁ is CH, R₁ is H and R₂ is H.

If Y is I 5 where W₁ is CH, Q₁ is O or S, Y₁₃ is selected from phenyl, 2-pyridyl, 3-pyridazinyl, 4-pyrimidinyl, 2-pyrimidinyl, 5-pyrimidinyl, 3-pyrazinyl, 2-thiophenyl, 2-furyl, 2-thiazolyl or 2-oxazolyl and X is —(CH₂)ₙ— where n=1-10 then R can not be I 8 where W₁ is CH, R₁ is H and R₂ is H.

If Y is 14 where Y₈ is O and Y₉ is N(Y₁₁)Y₁₂ and X is —(CH₂)ₙ— where n=0-3 then R can not be selected from I 8, I 9, I 10, I 11 and I 12.

If Y is I 4 where Y₈ is NH and Y₉ is N(Y₁₁)Y₁₂ where Y₁₁ is H and Y₁₂ is cyclohexyl and X is —(CH₂)ₙ— where n=0, then R can not be naphthyl.

Unless otherwise specifically defined, “alkyl” or “lower alkyl” refers to a linear, branched or cyclic alkyl group having from 1 to about 16 carbon atoms, and advantageously about 1 to about 6 carbon atoms, including, for example, methyl, ethyl, propyl, butyl, hexyl, octyl, isopropyl, isobutyl, tert-butyl, cyclopropyl, cyclohexyl, cyclooctyl, vinyl and allyl. Unless otherwise specifically defined, an alkyl group can be saturated or unsaturated. Unless otherwise specifically limited an alkyl group can be unsubstituted, singly substituted, or multiply substituted, with substituent groups in any possible position. Unless otherwise specifically defined, a lower alkyl group is a C1 to about C5 straight or branched chain alkyl group. Unless otherwise specifically limited, a cyclic alkyl group may include monocyclic, bicyclic, tricyclic, tetracyclic and polycyclic rings, for example norbornyl, adamantyl and related terpenes.

Unless otherwise specifically defined, “alkenyl” or “lower alkenyl” refers to a linear, branched or cyclic carbon chain having from 1 to about 16 carbon atoms, and advantageously about 1 to about 6 carbon atoms, and at least one double bond between carbon atoms in the chain. Examples include, for example, ethylene, allene, butene, butadiene, hexene, hexadiene, 5,5-dimethyl-1-hexene and cyclohexene. Unless otherwise specifically limited an alkenyl group can be unsubstituted, singly substituted, or multiply substituted, with substituent groups in any possible position.

Unless otherwise specifically defined, “alkynyl” or “lower alkynyl” refers to a linear, branched or cyclic carbon chain having from 1 to about 16 carbon atoms, and advantageously about 1 to about 6 carbon atoms, and at least one triple bond between carbon atoms in the chain. Examples include, for example, ethyne, butyne, and hexyne. Unless otherwise specifically limited an alkynyl group can be unsubstituted, singly substituted, or multiply substituted, with substituent groups in any possible position.

Unless otherwise specifically defined, “alkoxy” refers to the general formula —O-alkyl.

Unless otherwise specifically defined, “alkylamino” refers to the general formula —(NH)-alkyl.

Unless otherwise specifically defined, “di-alkylamino” refers to the general formula —N—(alkyl)₂. Unless otherwise specifically limited di-alkylamino includes cyclic amine compounds such as piperidine and morpholine.

Unless otherwise specifically defined, “aryl” refers to a polyunsaturated, aromatic hydrocarbon which can be a single ring or multiple rings (preferably from 1 to 3 rings) which are fused together or linked covalently and can include “divalent radicals”. The term “divalent aryl radicals” unless otherwise specifically defined refers to the general formula: -aryl-. Examples of aryl groups include but are not limited to, phenyl, biphenyl, napthyl. Unless otherwise specifically limited an aryl moiety can be unsubstituted, singly substituted, or multiply substituted, with substituent groups in any possible position.

Unless otherwise specifically defined, a bicyclic ring structure comprises 2 fused or bridged rings that include only carbon as ring atoms. The bicyclic ring structure may be saturated or unsaturated. Unless otherwise specifically limited a bicyclic ring structure can be unsubstituted, singly substituted, or multiply substituted, with substituent groups in any possible position. The individual rings may or may not be of the same type. Examples of bicyclic ring structures include, Dimethyl-bicyclo[3,1,1] heptane, bicyclo[2,2,1]heptadiene, decahydro-naphthalene and bicyclooctane.

Unless otherwise specifically defined, a carbocyclic ring is a non-aromatic ring structure having about 3 to about 8 ring members, substituted or unsubstituted, that includes only carbon as ring atoms, for example, cyclohexadiene or cyclohexane. Unless otherwise specifically limited a carbocyclic ring structure can be unsubstituted, singly substituted, or multiply substituted, with substituent groups in any possible position.

Unless otherwise specifically defined, “cycloalkyl” or “cycloalkyl ring” refers to a saturated ring structure having about 3 to about 8 ring members that has only carbon atoms as ring atoms and can include divalent radicals. The term “divalent cycloalkyl radicals” unless otherwise specifically defined refers to the general formula: -cycloalkyl-. Examples of cycloalkyl groups include but are not limited to, cyclopropyl, cyclobutyl, cyclopentyl, cyclohexyl.

A carbonyl group may exist in the hydrate form. Therefore, hydrates of the compounds are included in this disclosure.

Unless otherwise specifically defined, a cyclic glycerol includes members wherein 2 of the 3 hydroxy groups are tied to form a 5 to 8 member ring and the third hydroxyl group is substituted, for example in the form of an ester or an ether. The cyclic glycerol ring will typically, but not always, be saturated. The cyclic glycerol may be substituted in any possible position by one or more substituent groups. Examples of cyclic glycerols include

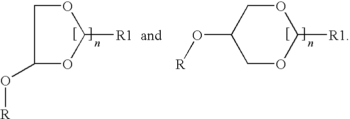

wherein n is an integer selected from 1 to 3.

Unless otherwise specifically defined, “halogen” refers to an atom selected from fluorine, chlorine, bromine and iodine.

Unless otherwise specifically defined, “heteroaryl” refers to aryl groups (or rings) that contain one or more heteroatoms selected from oxygen, nitrogen and/or sulfur as ring atoms. Heteroaryl groups (or rings) also include fused polycyclic systems in which one or more monocyclic aryl or monocyclic heteroaryl group is fused to another heteroaryl group. “Heteroaryl” can include “divalent radicals”, the term “divalent heteroaryl radicals” unless otherwise specifically defined refers to the general formula: -heteroaryl-. Examples of heteroaryl groups include but are not limited to, furanyl, thienyl, pyrrolyl, oxazolyl, thiazolyl, isoxazolyl, pyrazolyl, imidazolyl, oxadiazolyl, pyridinyl, pyrimidinyl, purinyl, benzothiazolyl, benzimibazolyl, benzofuranyl, indolyl, quinolinyl, quinoxalinyl. Unless otherwise specifically limited a heteroaromatic ring can be unsubstituted, singly substituted, or multiply substituted, with substituent groups in any possible position.

Unless otherwise specifically defined, a heterobicyclic ring structure comprises 2 fused or bridged rings that include carbon and one or more heteroatoms, including oxygen, nitrogen and/or sulfur, as ring atoms. The heterobicyclic ring structure is saturated or unsaturated. The heterobicyclic ring structure can be unsubstituted, singly substituted, or multiply substituted, with substituent groups in any possible position. The individual rings may or may not be of the same type. Examples of heterobicyclic ring structures include tropane, quinuclidine and tetrahydro-benzofuran.

Unless otherwise specifically defined, “heterocyclic” or “heterocyclic ring” refers to a saturated ring structure having about 3 to about 8 ring members that has carbon atoms and one or more heteroatoms, including oxygen, nitrogen and/or sulfur, as ring atoms. The term “heterocyclic” or “heterocyclic ring” can include “divalent radicals”. The term “divalent heterocyclic radicals” unless otherwise specifically defined refers to the general formula: -heterocyclic-. Examples of heterocyclic groups include but are not limited to, oxetane, thietane, azetidine, diazetidine, tetrahydrofuran, thiolane, pyrrolidine, dioxolane, oxathiolane, imidazolidine, dioxane, piperidine, morpholine, piperazine, and their derivatives. The heterocyclic ring can be unsubstituted, singly substituted, or multiply substituted, with substituent groups in any possible position.

Unless otherwise specifically defined, a heterotricyclic ring structure comprises 3 rings that may be fused, bridged or both, and that include carbon and one or more heteroatoms, including oxygen, nitrogen and/or sulfur, as ring atoms. The heterotricyclic ring structure can be saturated or unsaturated. The heterotricyclic ring structure can be unsubstituted, singly substituted, or multiply substituted, with substituent groups in any possible position. The individual rings may or may not be of the same type. Examples of heterotricyclic ring structures include 2,4,10-trioxaadamantane, tetradecahydro-phenanthroline.

Unless otherwise specifically defined, a polycyclic ring structure comprises more than 3 rings that may be fused, bridged or both fused and bridged and that include carbon as ring atoms. The polycyclic ring structure can be saturated or unsaturated. Unless otherwise specifically limited a polycyclic ring structure can be unsubstituted, singly substituted, or multiply substituted, with substituent groups in any possible position. The individual rings may or may not be of the same type. Examples of polycyclic ring structures include adamantine, bicyclooctane, norbornane and bicyclononanes.

Unless otherwise specifically defined, a tricyclic ring structure comprises 3 rings that may be fused, bridged or both fused and bridged and that includes carbon as ring atoms. The tricyclic ring structure can be saturated or unsaturated. The tricyclic ring structure can be unsubstituted, singly substituted, or if possible, multiply substituted, with substituent groups in any possible position. The individual rings may or may not be of the same type. Examples of tricyclic ring structures include fluorene and anthracene.

Unless otherwise specifically limited the term substituted means substituted by at least one below described substituent group in any possible position or positions. Substituent groups for the above moieties useful in the invention are those groups that do not significantly diminish the biological activity of the inventive compound. Substituent groups that do not significantly diminish the biological activity of the inventive compound include, for example, H, halogen, N₃, NCS, CN, NO₂, NX₁X₂, OX₃, C(X₃)₃, OAC, O-acyl, O-aroyl, NH-acyl, NH-aroyl, NHCOalkyl, CHO, C(halogen)₃, COOX₃, SO₃H, PO₃H₂, SO₂NX₁X₂, CONX₁X₂, COCF₃, alkyl, alcohol, alkoxy, alkylmercapto, alkylamino, di-alkylamino, sulfonamide or thioalkoxy wherein X₁ and X₂ each independently comprise H or alkyl, or X₁ and X₂ together comprise part of a heterocyclic ring having about 4 to about 7 ring members and optionally one additional heteroatom selected from O, N or S, or X₁ and X2 together comprise part of an imide ring having about 5 to about 6 members and X₃ comprises H, alkyl, loweralkylhydroxy, or alkyl-NX₁X₂. Unless otherwise specifically limited, a substituent group may be in any possible position or any possible positions if multiply substituted.

Unless otherwise specifically defined, advantageous substituents on a straight chain hydrocarbyl group include methyl, ethyl, hydroxy, hydroxymethyl, thiol, methoxy, ethoxy and hydroxy. Suitable substituents on an aryl, heteroaryl or heterocyclic group include groups such as lower alkyl, aryl, heteroaryl, (lower alkoxy)-O—, (aryl or substituted aryl)-O—, halo, —CO—O(lower alkyl), —CHO, —CO-(lower alkyl), —CO—NH(lower alkyl), —CO—N(lower alkyl)₂, —NO₂, —CF₃, —CN, and (lower alkyl)-S—.

The compounds of the present disclosure may have unnatural ratios of atomic isotopes at one or more of their atoms. For example, the compounds may be radiolabeled with isotopes, such as tritium or carbon-14. The present disclosure encompasses all isotopic variations of the described compounds, whether radioactive or not.

Having generally described the invention, the following examples are included for purposes of illustration so that the invention may be more readily understood and are in no way intended to limit the scope of the invention unless otherwise specifically indicated.

### Materials and Methods

Chemicals and antibodies. The cannabinoid compounds A, C, B, N-(morpholin-4-yl)-1-(2,4-dichlorophenyl)-5-(4-iodophenyl)-4-methyl-1H-pyrazole-3-carboxamide (H), and N-(piperidin-1-yl)-5-(4-iodophenyl)-1-(2,4-dichlorophenyl)-4-methyl-1H-pyrazole-3-carboxamide (G), were synthesized as previously described (Abadji et al., 1994; Beltramo et al., 1997; Deutsch et al., 1997; Lan et al., 1999, the contents of each of which are incorporated by reference herein). Glutamatergic ligands and antagonists were obtained from Tocris (Ellisville, Mo.). The monoclonal antibody against synaptophysin was obtained from Chemicon (Temecula, Calif.). Affinity-purified antibodies to GluR1 and to the calpain-mediated spectrin fragment BDP_{N} were also used (Bahr et al., 1995, 2002). Other antibodies utilized include those to the active form of ERK (Cell Signaling, Beverly, Mass.), active FAK (Upstate Biotechnology, Lake Placid, N.Y.), synapsin II (Cal-Biochem, San Diego, Calif.), and actin (Sigma, St. Louis, Mo.).
Hippocampal slice cultures. Sprague-Dawley rats (Charles River Laboratories; Wilmington, Mass.) were housed following guidelines from National Institutes of Health. As described previously (Bahr et al., 1995; Karanian et al., 2005, the contents of each of which are incorporated by reference herein), brains were rapidly removed at 11-12 days postnatal, and 400-μm hippocampal slices were positioned on Millicell-CM inserts (Millipore Corporation; Bedford, Mass.). The cultures were periodically supplied with fresh media consisting of basal medium Eagle (50%), Earle's balanced salts (25%), horse serum (25%), and defined supplements (Bahr et al., 1995 the contents of which are incorporated by reference herein). Slices were maintained in culture for a 15-20-day maturation period before experiments were initiated.
Activation of extracellular signal regulated-kinase (ERK) and focal adhesion kinase (FAK). Hippocampal slices were incubated for 30 min at 37° C. with 10-50 μM R-methanandamide (A), in the absence or presence of the CB1 antagonist H (10 μM). Other slices were treated with 100-500 nM B, 10-50 μM C, or a combination of the two in the absence or presence of either H or F (Cal-Biochem). The slices were then harvested in ice-cold buffer consisting of 0.32 M sucrose, 5 mM HEPES (pH 7.4), 1 mM EDTA, 1 mM EGTA, okadaic acid, 50 nM calyculin A, and a protease inhibitor cocktail containing 4-(2-aminoethyl)benzenesulfonyl fluoride, pepstatin A, E-64, bestatin, leupeptin, and aprotinin. Samples were homogenized in lysis buffer consisting of 15 mM HEPES (pH 7.4), 0.5 mM EDTA, 0.5 mM EGTA and the protease inhibitor cocktail. Protein content was determined and equal protein aliquots assessed by immunoblot for active pFAK with antibodies specific for its Tyr397 phosphorylation site, and for active pERK2 with antibodies specific for MEK-dependent phosphorylation sites in the catalytic core of ERK2 as described (Bahr et al., 2002; Karanian et al., 2005 the contents of each of which are incorporated by reference herein). Blots were routinely stained for a protein load control (e.g., actin). Anti-IgG-alkaline phosphatase conjugates were used for secondary antibody incubation and development of immunoreactive species was terminated prior to maximum intensity in order to avoid saturation. Integrated optical density of the bands was determined at high resolution with BIOQUANT software (R & M Biometrics, Nashville, Tenn.).
In vitro excitotoxicity. Cultured hippocampal slices were treated with 100 μM α-amino-3-hydroxy-5-methyl-isoxazole-4-propionic acid (AMPA) for 20 min. Immediately following the insult, AMPA was removed and the excitotoxic stimulation rapidly quenched with two 5-min washes containing glutamate receptor antagonists CNQX and MK801 as previously described (Bahr et al., 2002, the contents of which are incorporated by reference herein). The antagonists block any further activity of NMDA- and AMPA-type glutamate receptors, allowing for a controlled, reproducible excitotoxic insult. The slices were then incubated for 24 h with either 100 μM A or the drug combination of B/C (500 nM and 50 μM, respectively). At such time, slices were rapidly harvested, homogenized in lysis buffer, and analyzed by immunoblot for BDP_{N}, GluR1, and protein load control.
In vivo excitotoxicity. Adult Sprague-Dawley rats (175-225 g) were anesthetized with a solution of ketamine (10 mg/kg, IP) and xylazine HCl (0.2 mg/kg, IP). Using stereotaxic coordinates (−5.3 mm Bregma, −2.5 mm lateral), a 2.5-μl injection was administered to the right dorsal hippocampus (−2.9 mm from skull surface). Vehicle consisted of 50% DMSO in a phosphate-buffered saline solution. The insult included 63 nmol AMPA excitotoxin in the absence or presence of A (250 nmol) or the drug combination of B (0.75 nmol) and C (75 nmol). Following the injections, wounds were sutured and animals placed back in their home cage for recovery. After 4-7 days, brains were rapidly removed and either fixed in 4% paraformaldehyde or the dorsal hippocampal tissue dissected and snap frozen in dry ice. The hippocampal tissue was homogenized in lysis buffer, protein content determined, and assessed for cytoskeletal, synaptic, and signaling markers.

Histology. Brains fixed in paraformaldehyde were cryoprotected in 20% sucrose for 24 h. They were sectioned at 35-μm thickness using an American Optical AO860 precision sliding microtome (Buffalo, N.Y.), and mounted on Superfrost slides (Fisher, Pittsburgh, Pa.). Tissue was Nissl stained, dehydrated through ethanol solutions, and coverslipped.

Behavioral Testing. Animals that received intrahippocampal injections were assessed for motor changes 4-7 days after injection. Animals were placed in the center of a locomotor box during which time the rodents' gross motor movements were monitored using a photobeam activity system as described previously (Kosten et al., 2005, the contents of which are incorporated by reference herein). Total move time was assessed as the mean number of seconds in which the animal exhibits motion during, three 10-min sessions. A separate study evaluated memory function using a fear conditioning paradigm slightly modified from one previously described (Kosten et al., 2005, the contents of which are incorporated by reference herein). Briefly, the rodents were placed in a chamber for 3 min and then presented with 7 pairings, each 1 min apart, of a 10-sec tone (2.9 kHz, 82 dB) that co-terminated with a 1-sec foot shock (1 mA). Post training, animals received intrahippocampal injections of vehicle, AMPA, or AMPA with B/C. After 4-7 days, the rodents were placed back in the chamber and baseline movement assessed for 3 min. Freezing behavior (inactivity for ≧3 sec) was then monitored as tone was delivered 7 times, each 1 min apart.

Statistical analyses. Mean integrated densities for antigens and separate groups of behavioral data were evaluated using ANOVA and Tukey's post-hoc tests.

### Results

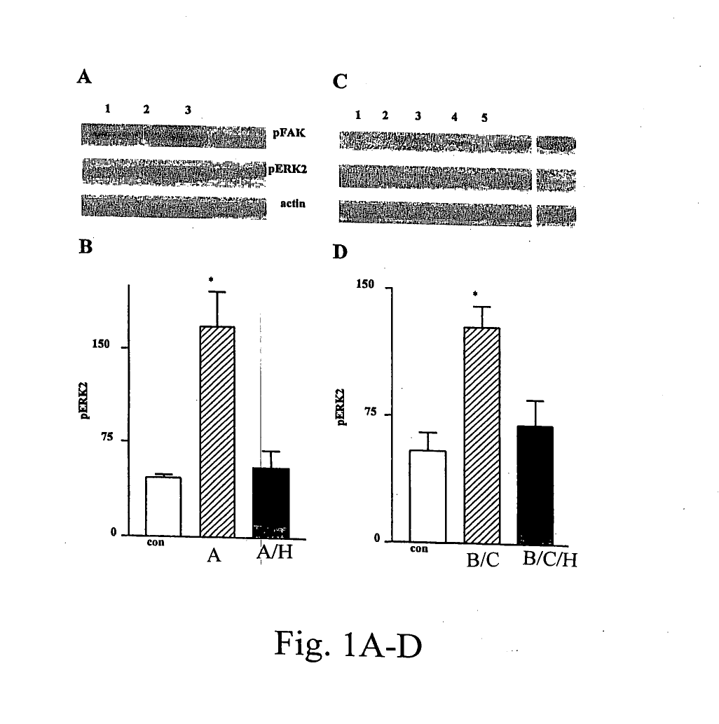

B and C Promote CB1 Signaling. To test whether inhibitors of endocannabinoid transport and FAAH enhance CB1 receptor responses, we used hippocampal slice cultures prepared from rats at postnatal day 12-13. The cultured slices express compensatory responses to injury and survival signaling pathways that are similar to those found in the adult brain (Bahr et al., 2002; Khaspekov et al., 2004; Karanian et al., 2005, the contents of each of which are incorporated by reference herein). As shown in FIGS. 1A and 1B, stimulation of CB1 receptors with the stable agonist R-methanandamide (A) results in the activation of extracellular signal regulated-kinase (ERK) as well as focal adhesion kinase (FAK), a signaling event upstream of the ERK/MAPK pathway (Derkinderen et al., 1998; Karanian et al., 2005, the contents of each of which are incorporated by reference herein). The phosphorylated active forms of FAK (pFAK) and the ERK2 isoform (pERK2) were increased over basal levels expressed by control slices.

Activation of ERK and FAK also occurred when 100 nM of the FAAH inhibitor B and 10 μM of the transport inhibitor C were applied individually to the cultures (FIG. 1C). Lower concentrations of the inhibitors were less effective at activating the signaling pathways. As a control, actin was found not to change with drug treatment, and total FAK and total ERK levels were previously shown to be unaffected. Interestingly, blocking both mechanisms of endocannabinoid inactivation with the B/C combination resulted in a surprisingly greater effect, producing a comparable level of cannabinergic signaling as that produced by the agonist A. The drug combination increased pFAK by 140-190% and pERK2 by 60-100% over those levels activated by the individual drugs. Also shown in FIG. 1C (lane 5), inhibiting MAPK's upstream activator MEK caused selective blockage of B/C-mediated ERK activation, as previously reported for agonist-induced ERK activation (Karanian et al., 2005, the contents of which are incorporated by reference herein). The effects of agonist treatment (FIG. 1B) as well as endocannabinoid potentiation by B/C (FIG. 1D) were prevented by the CB1 antagonist H. The data indicate that indirect pharmacological modulation of the endocannabinoid system is an efficient strategy to promote signaling through CB1 receptors.

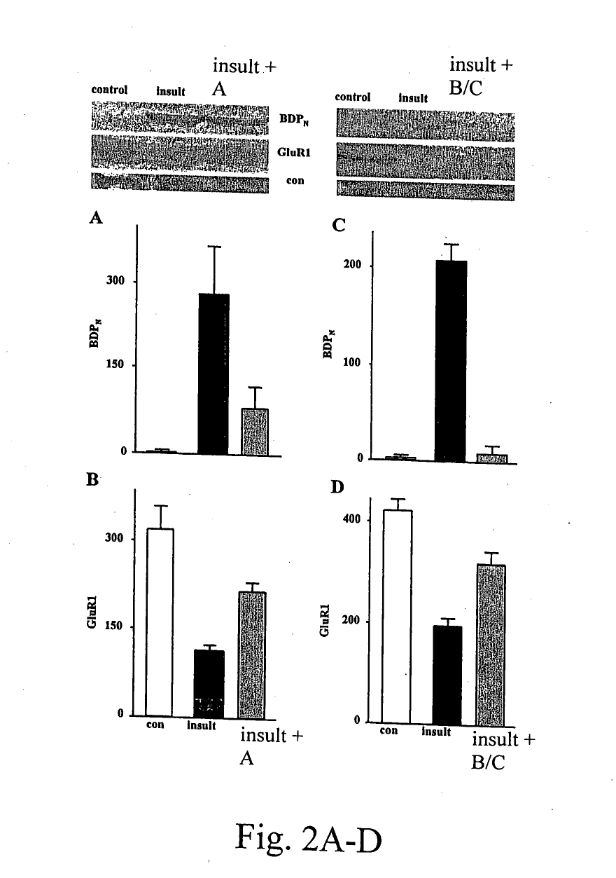

Neuroprotection In Vitro. Indirect endocannabinoid potentiation with B/C was tested for protective features in the excitotoxic hippocampal slice model, and compared to the actions of a CB1 agonist. The slice cultures were subjected to excitotoxic stimulation of AMPA-type glutamate receptors for 20 min, resulting in persistent cytoskeletal damage indicated by the calpain-mediated spectrin breakdown product BDP_{N} evident 24 h later. Post-insult activation of cannabinoid responses with the agonist A reduced the cytoskeletal damage by 72% (FIG. 2A). Note that we also found, as shown by others (Shen and Thayer, 1998, the contents of which are incorporated by reference herein), that the cannabinergic system is effective against the over-activation of NMDA receptors. Here, NMDA-induced BDP_{N} levels (231±39; mean±SEM, n=8) were significantly reduced by activating CB1 receptors (89±19; n=8, P<0.01).

In the AMPA-treated slice cultures, the cytoskeletal breakdown marker was also reduced by the B/C combination, in this case by 96% (FIG. 2C). Note that excitotoxic calpain activation is commonly associated with reductions in synaptic markers (see Riederer et al., 1992; Vanderklish and Bahr, 2000; Bahr et al., 2002; Munirathinam et al., 2002; Karanian et al., 2005, the contents of each of which are incorporated by reference herein), and across slice samples, A (FIG. 2B) and B/C (FIG. 2D) both attenuated the loss of the postsynaptic marker GluR1 by 50-70% (ANOVAs: P<0.01). Thus, dual blockage of endocannabinoid inactivation with B and C produced the same level of excitotoxic protection in vitro as that produced by the direct activation of CB1 receptors. When applied separately, B and C were surprisingly less effective with regards to cytoskeletal and synaptic protection (see Table 1).

TABLE 1

| drug treatment | reduction in BDP_{N} | recovery of GluR1 |
|---|---|---|
| B | 36 ± 12% | 25 ± 6% |
| C | 22 ± 7% | 20 ± 6% |
| B/C | 96 ± 8%* | 56 ± 5%* |

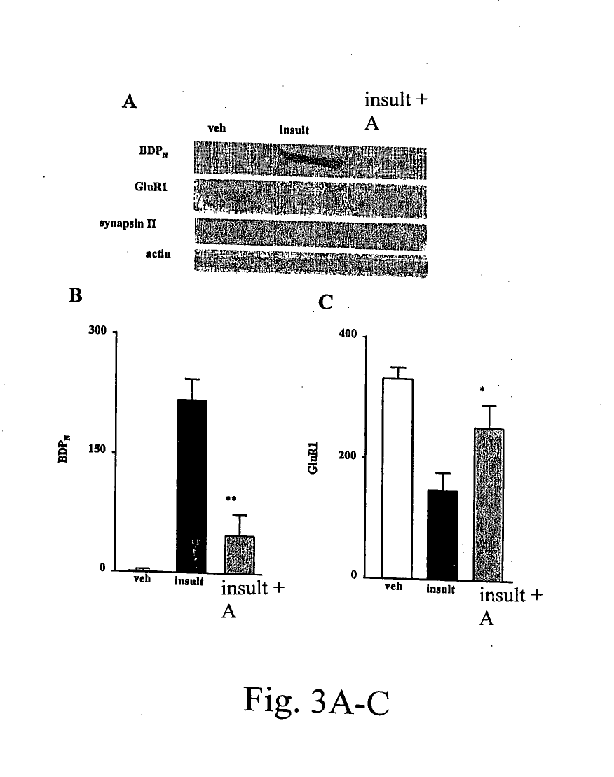

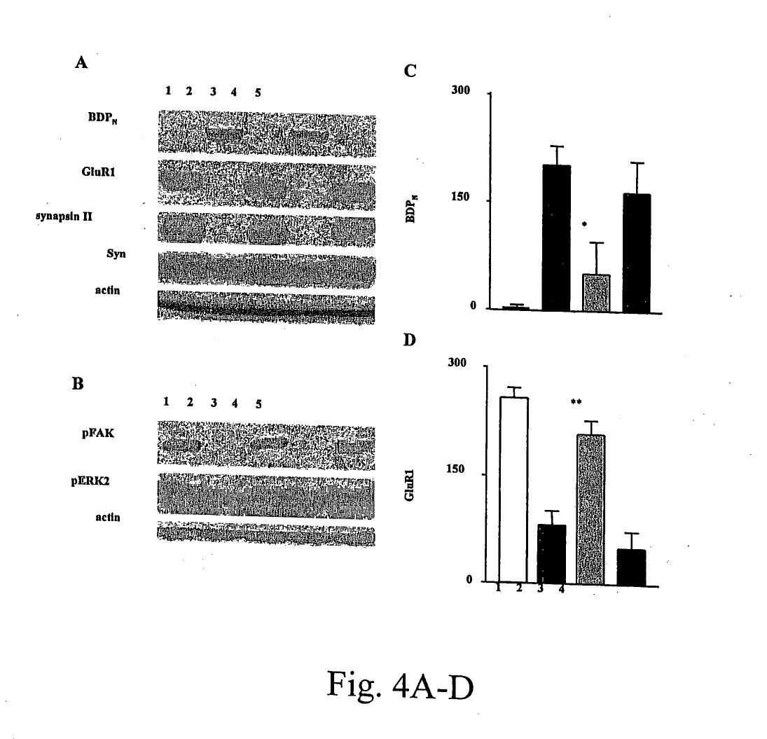

Table 1. Improved cytoskeletal and synaptic protection with dual blockage of endocannabinoid inactivation. Slice cultures were subjected to an AMPA insult as in FIG. 2, then received B (0.5 μM), C (50 μM), or a combination of the two drugs. Slices harvested 24 h post-insult were assessed for BDP_{N} and GluR1, and percent changes from insult alone slices were determined (ANOVAs: P=0.001 and 0.003, respectively). Mean percentages ±SEM are listed (n=5-8). Post-hoc tests compared to individual drug data: *P<0.01.
Neuroprotection In Vivo. The B/C drug combination and the A agonist were tested in an in vivo model of excitotoxic brain damage. In order to initiate excitotoxicity in adult rats, 63 nmol of AMPA were injected unilaterally into the dorsal hippocampus. The dorsal half of the hippocampus was dissected 4-7 days later, and the ipsilateral tissue exhibited a pronounced level of cytoskeletal breakdown as well as reductions in pre- and postsynaptic markers (FIG. 3A). As in the slice model, the excitotoxic cytoskeletal breakdown in vivo correlated with synaptic decline (r=−0.74, P<0.0001), and cannabinoid responses were protective against the pathogenic manifestations. When A was co-injected with the AMPA insult, spectrin BDP_{N} evident 4-7 days post-insult was reduced by 78% (FIG. 3B). Similar reduction in cytoskeletal damage was found when endocannabinoid inactivation was blocked with B/C during the excitotoxic insult (FIG. 4C). In fact, the drug combination provided what appeared to be complete cytoskeletal protection in 9 of the 12 animals examined.

In addition to the protective effects on cytoskeletal integrity, A reduced the postsynaptic GluR1 decline evident in the excitotoxic animals by an average of 58% (FIG. 3C). The presynaptic marker synapsin II was protected by a similar level (see FIG. 3A). As with cytoskeletal protection, blocking endocannabinoid inactivation provided a similar if not higher degree of synaptic protection as did the CB1 receptor agonist. The B/C combination protected GluR1 levels by an average of 74% (FIG. 4D). When individual animals were assessed, 8 of the 12 that received B/C exhibited >90% preservation of the postsynaptic marker. Also, the presynaptic markers synapsin II and synaptophysin were almost completely protected by the drug combination (see FIG. 4A). B/C' s effects on cytoskeletal (FIG. 4C) and synaptic protection (FIG. 4D) were blocked by the CB1 antagonist G, indicating that the effects were mediated through CB1 receptors. These results indicate that direct and indirect activation of CB1 signaling provides similar protection in vivo.

Interestingly, blocking endocannabinoid transport and hydrolysis not only produced cytoskeletal and synaptic protection against the AMPA insult, it also maintained important signaling pathways. Basal levels of activated pFAK and pERK were maintained by B/C at levels found in vehicle-treated animals (compare lane 3 to control lane 1 in FIG. 4B). In addition, the G antagonist correspondingly abolished the effects of B/C on cytoskeletal protection, synaptic protection, and the maintenance of FAK and ERK/MAPK pathways (lane 4 in FIGS. 4A and 4B). Pathogenic changes remained similar to those found with insult alone despite the co-injection of B/C, thus the antagonist eliminated any indication of protection and did so without worsening the excitotoxic damage. None of the antigens tested were altered when the single G injection was administered alone (lane 5), and the lack of cytoskeletal damage or synaptic decline confirmed that no toxicity is involved. Together, the results strongly suggest that the neuroprotection by B/C is mediated through CB1 receptor responses.

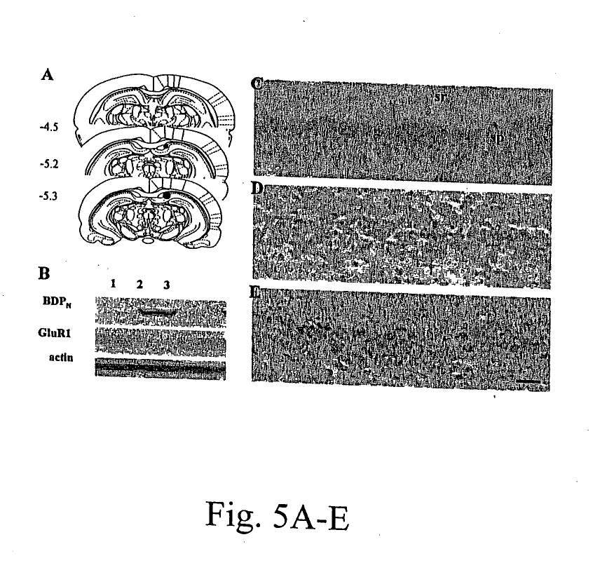

To confirm cellular protection by the B/C combination, brains from the different treatment groups were rapidly dissected at 7 days post-insult, then fixed and sectioned for staining by Nissl. Coronal sections confirmed the unilateral damage at the excitotoxin injection site in the dorsal hippocampus (FIG. 5A). In addition, the contralateral hippocampus had no evidence of spectrin BDP_{N} or associated synaptic decline as found in the ipsilateral tissue (FIG. 5B). The AMPA insult also caused a pronounced decrease in density of CA1 pyramidal neurons in the ipsilateral hippocampus, as well as an increase in pyknotic nuclei (FIG. 5D) as compared to tissue from control rats (FIG. 5C). Previous studies have reported a correspondence between calpain-mediated spectrin breakdown and subsequent cell death in the hippocampal CA1 subfield. Accordingly, the B/C drug combination that reduced excitotoxic cytoskeletal breakdown was found to prevent neuronal death and pyknotic changes (FIG. 5E).

Functional Protection. Two behavioral correlates of neuronal damage were employed to test the B/C combination for functional protection. The first involves perseverative turning shown to be induced by AMPA injections into the brain and by selective hippocampal damage (see Mickley et al., 1989; Smith et al., 1996, the contents of each of which are incorporated by reference herein). As shown in the left data set of FIG. 5A, intrahippocampal AMPA injections were found associated with steady, slow bouts of turning assessed 4-7 days post-insult. The excitotoxic brain damage caused a 4-6-fold increase over the basal turning exhibited by vehicle-injected control animals. The increased turning was reduced to near control levels when endocannabinoid inactivation was disrupted by co-injecting B and C with the excitotoxin (ANOVA: p=0.0017). The reduction in turning was more pronounced than that produced by the agonist A (not shown). Although enhancing cannabinoid responses would be expected to reduce motor behavior, this is not likely the case here since animals were assessed for exploratory movement several days after drugs were administered. In addition, total move time in the locomotor box per session was not changed in drug-treated animals or by the insult itself (right data set in FIG. 5A). Thus, assessment of a selective behavioral disturbance further supports that the B/C combination protects against excitotoxic brain damage.

The second behavioral correlate of brain damage measured was memory impairment. A fear conditioning paradigm was used since this learning task is sensitive to electrolytic and excitotoxic lesions in the dorsal hippocampus (Anagnostaras et al., 1999; Zou et al., 1999, the contents of each of which are incorporated by reference herein). Rats were trained to fear an innocuous stimulus, in this case consisting of both the context of an operant chamber and a tone. Seven pairings of a 10-sec tone with an aversive foot shock were given 2-2.5 hours before the unilateral AMPA injection into the dorsal hippocampus. When re-exposed to the conditional environment/tone 4-7 days later, control rats exhibited the adaptive fear response of freezing while AMPA-injected rats did not (FIG. 5B, left data set). Thus, the excitotoxic damage decreased fear conditioning likely by impairing consolidation processes of the hippocampus. As evident in FIG. 5B, endocannabinoid inactivation with the B/C combination protected such hippocampal processes and, as a result, significantly reduced the memory impairment causing improved fear responses (ANOVA: P<0.0001). The rats demonstrated storage of the conditioning chamber and tone, and these are the same animals that exhibited pronounced reduction in spectrin breakdown product and recovery of synaptic markers. Moreover, reduced BDP_{N} levels exhibited a significant correlation with improved performance in the fear conditioning task (r=−0.68, P<0.01) as did increased levels of the postsynaptic protein GluR1 (r=0.73, P<0.01). The CB1 antagonist G blocked the B/C-mediated functional protection (FIG. 5B), corresponding with its blockage effects on cytoskeletal and synaptic protection. As a control, baseline locomotor activity was determined in a subset of animals during a time period immediately before testing for memory storage. Baseline freezing was characteristically low and did not differ across the treatment groups (right data set in FIG. 5B). Taken together, these results demonstrate that indirect endocannabinoid potentiation protects against excitotoxic hippocampal damage that disrupts memory consolidation and/or recall.

### Synthesis

### Synthesis of Some of the Compounds of Structural Formula VI.

### 1. Synthesis of Sulfonyl Fluorides

Phenylalkylsulfonyl fluorides 4.1, 4.2, and 4.3 (shown in Scheme 1) were synthesized by a method depicted in Scheme 1 starting from commercially available phenylalkyl alcohols 1.1, 1.2, and 1.3.

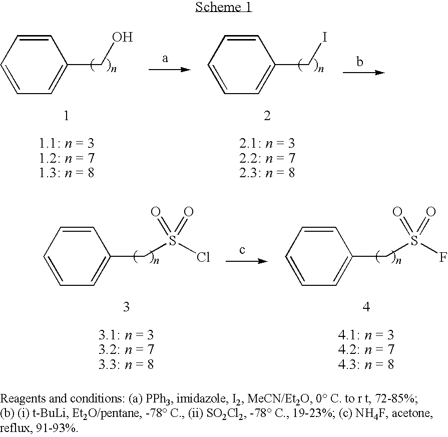

### Experimental Procedures

### Phenylalkyl Iodides (2)

A round bottom flask was charged with phenylalkyl alcohol 1 (1 equiv.), acetonitrile/diethyl ether mixture (1:2), triphenyl phosphine (1.3 equiv.), imidazole (1.3 equiv.), and iodine (1.3 equiv.). The solution was blanketed with argon and capped and the reaction stirred for 4-5 hours at room temperature. The resulting mixture diluted with diethyl ether, washed with water, aqueous sodium thiosulfate, and brine, dried (MgSO₄) and evaporated. Purification by flash column chromatography on silica gel (10% diethyl ether-hexane) gave phenylalkyl iodide 2 in 72-85% yield.

### Phenylalkylsulfonyl Chlorides (3)

A solution of phenylalkyl iodide 2 (1 equiv.) in a mixture of dry n-pentane/diethyl ether (3:2) was cooled to −78° C. under argon, and t-BuLi (2.2 equiv., using a 1.7 M solution of t-BuLi in hexane) was added dropwise over a 2-min period. The mixture was stirred for 10 min at −78° C. and then was transferred by cannula to a cooled (−78° C.) and dry solution of SO₂Cl₂ in n-pentane over a 20-min period. Following the addition, the reaction mixture was stirred for 1 hour at −78° C. and then allowed to warm to room temperature over a 3 hours period. The reaction mixture was quenched with dropwise addition of water, then diluted with diethyl ether and the organic phase was separated. The aqueous phase was extracted with diethyl ether, the combined organic layer was dried (MgSO₄) and the solvent was evaporated. Purification by flash column chromatography on silica gel gave phenylalkylsulfonyl chloride 3 in 19-23% yield.

### Phenylalkylsulfonyl Fluorides (4)

To a stirred solution of phenylalkylsulfonyl chloride 3 (1 equiv.) in dry acetone, was added anhydrous NH₄F (2 equiv.) and the mixture refluxed for 2 hours. The reaction mixture was cooled to room temperature, the solvent was evaporated, and the residue obtained was dissolved in diethyl ether. The ethereal solution was successively washed with water and brine, dried (MgSO₄) and concentrated under reduced pressure. Purification by flash column chromatography on silica gel gave phenylalkylsulfonyl fluoride 4 in 91-93% yield.

### Selected Data of Synthesized Phenylalkylsulfonyl Fluorides (4)

3-Phenyl-propanesulfonyl fluoride (4.1). ¹H NMR (200 MHz, CDCl₃) δ 7.46-7.15 (m, 5H), 3.40-3.27 (m, 2H), 2.82 (t, J=7.3 Hz, 2H), 2.40-2.21 (m, 2H); mass spectrum m/z (relative intensity) 202 (M⁺, 27), 91 (100).

7-Phenyl-heptanesulfonyl fluoride (4.2). Mass spectrum m/z (relative intensity) 258 (M⁺, 10), 105 (9), 91 (100).

8-Phenyl-octanesulfonyl fluoride (4.6). ¹H NMR (200 MHz, CDCl₃) δ 7.45-7.05 (m, 5H), 3.40-3.25 (m, 2H), 2.60 (t, J=7.1 Hz, 2H), 2.10-1.20 (m, 12H).

Sulfonyl fluorides 13.1, 13.2, 13.3, 13.4, 14.1, 14.2, 14.3, 14.4 (shown in Scheme 2) were synthesized by a method depicted in Scheme 2 starting from commercially available 2- or 3- or 4-anisaldehyde and the appropriate phenoxyalkyl bromide.

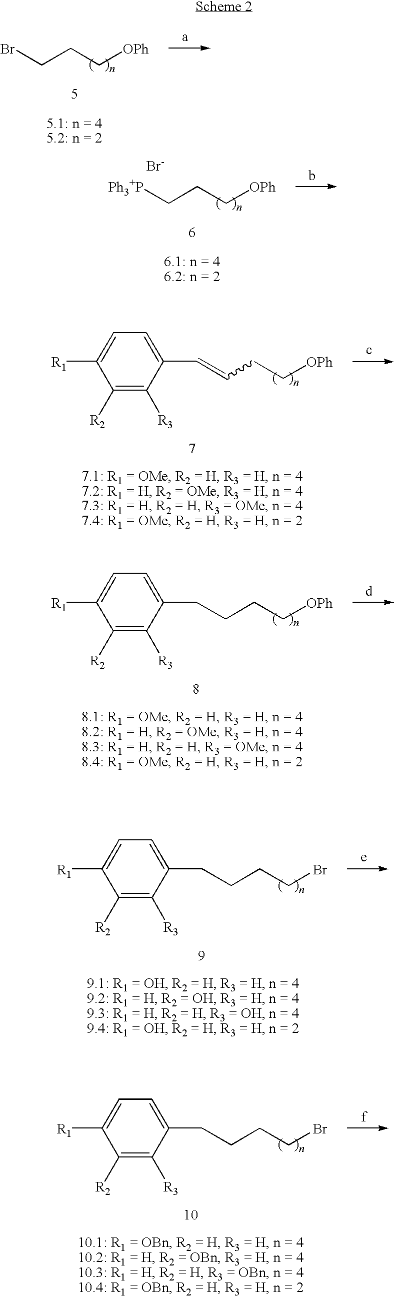

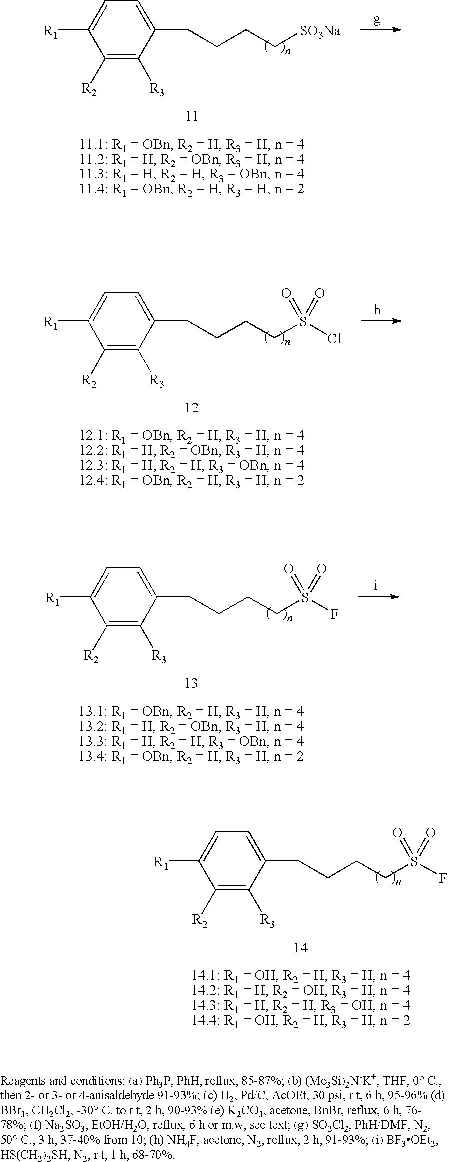

### Experimental Procedures

### 6-Phenoxyhexyltriphenylphosphonium bromide 6.1

A mixture of 6-phenoxyhexyl bromide 5.1 (2.8 g, 10.9 mmol) and triphenylphosphine (314 g, 12 mmol) in anhydrous benzene (100 mL), under an argon atmosphere, was refluxed for two days. The reaction mixture was allowed to cool to room temperature and the precipitating product (6.1) was isolated by filtration under reduced pressure and washed with anhydrous diethyl ether (4.75 g, 84% yield). White solid, m p 143-145° C. ¹H NMR (500 MHz, CDCl₃) δ 7.89-7.85 (m as dd, 6H), 7.81-7.75 (m as td, 3H), 7.71-7.67 (m as td, 6H), 7.25 (t, J=7.7 Hz, 2H), 6.91 (t, J=7.7 Hz, 1H), 6.84 (d, J=7.7 Hz, 2H) 3.95-3.85 (m and t overlapping, especially 3.90, t, J=6.3 Hz, 4H), 1.79-1.65 (m, 6H), 1.49 (quintet, J=7.7 Hz, 2H).

### 4-Phenoxybutyltriphenylphosphonium bromide 6.2

The title compound was synthesized as in 6.1 using 4-phenoxybutyl bromide (5.2) (22.0 g, 95.9 mmol) and triphenylphosphine (27.6 g, 105.5 mmol) in anhydrous benzene (50 mL), to give 6.1 (40.0 g, 85% yield). White solid, m p 185-186° C.

¹H NMR (500 MHz, CDCl₃) δ 7.88-7.84 (m as dd, 6H), 7.78-7.76 (m as td, 3H), 7.68-7.65 (m, 6H), 7.25 (t, J=7.7 Hz, 2H), 6.92 (t, J=7.7 Hz, 1H), 6.82 (d, J=7.7 Hz, 2H), 4.09 (t, J=4.5 Hz, 2H), 4.04-3.98 (m, 2H), 2.25 (quintet, J=6.4 Hz, 2H), 1.92-1.86 (m, 2H).

### 1-(4-Methoxyphenyl)-7-phenoxy-1-heptene (7.1)

To a suspension of 6-phenoxyhexyltriphenylphosphonium bromide (6.1) (4.60 g, 8.86 mmol) in dry THF (80 mL) at 0° C., under an argon atmosphere was added potassium bis(trimethylsilyl)amide (1.76 g, 8.86 mmol). The resulting slurry was stirred for 5 min at the same temperature and then a solution of 4-methoxybenzaldehyde (0.61 g, 4.46 mmol) in dry THF (10 mL) was added. The reaction mixture was stirred for an additional 10 min and quenched with saturated aqueous NH₄Cl (20 mL). The resulting mixture was warmed to room temperature, diluted with Et₂O (100 mL), the organic phase was separated and the aqueous phase extracted with Et₂O. The combined organic layer was washed with brine, dried over MgSO₄ and the solvent evaporated under reduced pressure. The residue obtained was purified through a short column of silica gel, eluting with 5% Et₂O-hexane, to give the product 7.1 (1.21 g, 92% yield, predominantly cis, cis:trans=96:4) as a colorless liquid.

¹H NMR (500 MHz, CDCl₃) δ 7.27 (t, J=7.5 Hz, 2H), 7.21 (d, J=8.7 Hz, 2H), 6.92 (t, J=7.5 Hz, 1H), 6.91-6.86 (m, overlapping signals, 4H), 6.35 (d, J=11.5 Hz, 1H), 5.57 (dt, J=11.5 Hz, J=7.5 Hz, 1H), 3.94 (t, J=6.0 Hz, 2H), 3.81 (s, 3H), 2.41-2.20 (m, 2H), 1.78 (quintet, J=6.7 Hz, 2H), 1.58-1.48 (m, 4H).

1-(3-Methoxyphenyl)-7-phenoxy-1-heptene (7.2) was synthesized as described in 7.1 using 6.1 (3.20 g 6.16 mmol), dry THF (30 mL), potassium bis(trimethylsilyl)amide (1.23 g, 6.16 mmol), and 3-methoxybenzaldehyde (0.28 g, 2.05 mmol). The title compound (7.2) was isolated as a colorless liquid after purification by flash column chromatography (0.564 g, 93% yield, predominantly cis, cis:trans=95:5).

¹H NMR (500 MHz, CDCl₃) δ 7.27-7.21 (m, 3H), 6.92 (t, J=7.0 Hz, 1H), 6.90-6.86 (m, 3H), 6.81 (t, J=1.5 Hz, 1H), 6.78 (dd, J=8.5 Hz, J=1.5 Hz, 1H), 6.39 (d, J=11.7 Hz, 1H), 5.67 (dt, J=11.7 Hz, J=7.5 Hz, 1H), 3.94 (t, J=6.5 Hz, 2H), 3.80 (s, 3H), 2.37 (q, J=6.5, 2H), 1.78 (quintet, J=6.5 Hz, 2H), 1.56-1.48 (m, 4H).

1-(2-Methoxyphenyl)-7-phenoxy-1-heptene (7.3) was synthesized as described in 7.1 using 6.1 (2.0 g, 3.85 mmol), dry THF (30 mL), potassium bis(trimethylsilyl)amide (0.77 g, 3.85 mmol), and 2-methoxybenzaldehyde (0.20 g, 1.47 mmol). The title compound (7.3) was isolated as a colorless liquid after purification by flash column chromatography (0.396 g, 91% yield, predominantly cis, cis:trans=93:7).

¹H NMR (500 MHz, CDCl₃) δ 7.29-7.21 (m, 4H), 6.94-6.87 (m, 5H), 6.52 (d, J=11.2 Hz, 1H), 5.73 (dt, J=11.2 Hz, J=7.5 Hz, 1H), 3.93 (t, J=6.7 Hz, 2H), 3.83 (s, 3H), 2.28 (m as q, J=7.2 Hz, 2H), 1.76 (quintet, J=7.2 Hz, 2H), 1.53-1.46 (m, 4H).

1-(4-Methoxyphenyl)-7-phenoxy-1-pentene (7.4) was synthesized as described in 7.1 using 6.2 (29.0 g, 58.8 mmol), dry THF (200 mL), potassium bis(trimethylsilyl)amide (11.7 g, 58.8 mmol) and 4-methoxybenzaldehyde (2.9 g, 14.7 mmol). The title compound (7.4) was isolated as a colorless liquid after purification by flash column chromatography (3.69 g, 93% yield, predominantly cis, cis:trans=96:4).

¹H NMR (500 MHz, CDCl₃) δ 7.26 (t, J=7.5 Hz, 2H), 7.22 (d, J=8.7 Hz, 2H), 6.92 (t, J=7.5 Hz, 1H), 6.87 (d, J=7.5 Hz, 2H), 6.85 (d, J=8.7 Hz, 2H), 6.39 (d, J=11.5 Hz, 1H), 5.60 (dt, J=11.5 Hz, J=7.0 Hz, 1H), 3.98 (t, J=6.0 Hz, 2H), 3.80 (s, 3H), 2.51 (m as qd, J=7.5 Hz, J=2.1 Hz, 2H), 1.94 (quintet, J=6.7 Hz 2H).

### 1-(4-Methoxyphenyl)-7-phenoxy-heptane (8.1)

To a stirred solution of 7.1 (1.19 g, 4.03 mmol) in AcOEt (40 mL) at room temperature was added 10% Pd/C (0.18 g, 15% w/w) and the resulting suspension was hydrogenated (30 psi, 6 h). The catalyst was removed by filtration through celite and the filtrate was evaporated under reduced pressure to give the title compound (8.1) (1.14 g, 95% yield) as a white solid (m p 32-34° C.).

¹H NMR (500 MHz, CDCl₃) δ 7.30 (t, J=8.5 Hz, 2H), 7.11 (d, J=8.2 Hz, 2H), 6.95 (t, J=8.5 Hz, 1H), 6.92 (d, J=8.5 Hz 2H), 6.84 (d, J=8.2 Hz, 2H), 3.97 (t, J=6.7 Hz, 2H), 3.81, (s, 3H) 2.57 (t, J=7.5 Hz, 2H), 1.78 (quintet, J=6.7 Hz, 2H), 1.62 (quintet, J=7.5 Hz, 2H), 1.48 (quintet, J=7.5 Hz, 2H), 1.44-1.34 (m, 4H).

1-(3-Methoxyphenyl)-7-phenoxy-heptane (8.2) was synthesized as described in 8.1 using 7.2 (0.55 g, 1.86 mmol), AcOEt (20 mL), and 10% Pd/C (0.080 g, 15% w/w). The title compound (8.2) was isolated as a colorless viscous liquid (0.53 g, 96% yield).

¹H NMR (500 MHz, CDCl₃) δ 7.27 (t, J=_{7.0} Hz, 2H), 7.19 (t, J=7.4 Hz, 1H), 6.92 (t, J=7.0 Hz, 1H), 6.89 (d, J=7.0 Hz, 2H), 6.77 (d, J=7.4 Hz, 1H), 6.73-6.71 (m, 2H), 3.94 (t, J=6.5 Hz, 2H), 3.79 (s, 3H), 2.58 (t, J=7.5 Hz, 2H), 1.77 (quintet, J=6.7 Hz, 2H), 1.62 (quintet, J=7.2 Hz, 2H), 1.50-1.42 (m, 2H), 1.42-1.34 (m, 4H).

1-(2-Methoxyphenyl)-7-phenoxy-heptane (8.3) was synthesized as described in 8.1 using 7.3 (0.35 g, 1.18 mmol), AcOEt (20 mL), and 10% Pd/C (0.050 g, 14% w/w). The title compound (8.3) was isolated as a colorless viscous liquid (0.33 g, 95% yield).

¹H NMR (500 MHz, CDCl₃) δ 7.27 (t, J=7.5 Hz, 2H), 7.16 (t; J=7.5 Hz, 1H), 7.12 (d, J=7.5 Hz, 1H), 6.94-6.83 (m, 5H), 3.95 (t, J=6.5 Hz, 2H), 3.81 (s, 3H), 2.60 (t, J=7.7, 2H), 1.78 (quintet, J=7.0 Hz, 2H), 1.59 (quintet, J=7.0 Hz, 2H), 1.48-1.43 (m, 2H), 1.42-1.38 (m, 4H).

1-(4-Methoxyphenyl)-5-phenoxy-pentane (8.4) was synthesized as described in 8.1 using 7.4 (3.67 g, 13.69 mmol), AcOEt (100 mL), and 10% Pd/C (0.550 g, 15% w/w). The title compound (8.3) was isolated as a white solid (m p 32-34° C.) in 95% yield (3.52 g).

¹H NMR (500 MHz, CDCl₃) δ 7.27 (t, J=7.5 Hz, 2H), 7.09 (d, J=8.5 Hz, 2H), 6.92 (t, J=7.5 Hz, 1H), 6.88 (d, J=7.5 Hz, 2H), 6.82 (d, J=8.5 Hz, 2H), 3.94 (t, J=6.5 Hz, 2H), 3.78 (s, 3H), 2.58 (t, J=7.7 Hz, 2H), 1.80 (quintet, J=6.7 Hz, 2H), 1.66 (quintet, J=7.0 Hz, 2H), 1.49 (quintet, J=7.5 Hz, 2H).

### 7-Bromo-1-(4-hydroxy-phenyl)-heptane (9.1)

To a stirred solution of 8.1 (1.1 g, 3.69 mmol) in anhydrous CH₂Cl₂, (40 mL), at −30° C., under an argon atmosphere was added BBr₃ (8 mL, 8 mmol, using an 1M solution in CH₂Cl₂) and the mixture gradually warmed to room temperature (2 h). Unreacted boron tribromide was destroyed by addition of aqueous saturated NaHCO₃ solution (10 mL) to the reaction mixture at 0° C. The resulting mixture was warmed to room temperature and diluted with Et₂O (40 mL). The organic layer was separated and the aqueous phase extracted with Et₂O. The combined organic layer was washed with brine, dried over MgSO₄ and the solvent evaporated under reduced pressure. The residue obtained was chromatographed through a short column of silica gel, eluting with 20% Et₂O-hexane to give 8.1 (0.930 g, 93% yield) as a viscous liquid.

¹H NMR (500 MHz, CDCl₃) δ 7.03 (d, J=8.5 Hz, 2H), 6.74 (d, J=8.5 Hz, 2H), 4.59 (br s, 1H), 3.34 (t, J=6.7 Hz, 2H), 2.53 (t, J=7.7 Hz, 2H), 1.84 (quintet, J=7.0 Hz, 2H), 1.57 (quintet, J=7.5 Hz, 2H), 1.46-1.38 (m, 2H), 1.36-1.31 (m, 4H).

7-Bromo-1-(3-hydroxy-phenyl)-heptane (9.2) was synthesized as in 9.1 using 8.2 (0.50 g, 1.68 mmol), in anhydrous CH₂Cl₂(16 mL), and BBr₃ (1M solution in CH₂Cl₂, 3.7 mL, 3.7 mmol). The title compound (9.2) was isolated as a viscous liquid after purification by flash column chromatography (0.420 g, 92% yield).

¹H NMR (500 MHz, CDCl₃) δ 7.14 (t, J=8.0 Hz, 1H), 6.75 (d, J=8.0 Hz, 1H), 6.66-6.63 (d and dd overlapping, 2H), 4.67 (br s, 1H), 3.40 (t, J=6.7 Hz, 2H), 2.56 (t, J=7.7 Hz, 2H), 1.85 (quintet, J=7.0 Hz, 2H), 1.62 (quintet, J=7.5 Hz, 2H), 1.46-1.38 (m, 2H), 1.36-1.32 (m, 4H).

7-Bromo-1-(2-hydroxy-phenyl)-heptane (9.3) was synthesized as in 9.1 using 8.3 (0.30 g, 1.01 mmol) in anhydrous CH₂Cl₂ (10 mL), and BBr₃ (1M solution in CH₂Cl₂, 2.2 mL, 2.2 mmol). The title compound (9.3) was isolated as a viscous liquid after purification by flash column chromatography (0.247 g, 90% yield).

¹H NMR (500 MHz, CDCl₃) δ 7.11 (dd, J=7.5 Hz, J=1.5 Hz, 1H), 7.07 (td, J=7.5 Hz, J=1.5 Hz, 1H), 6.87 (td, J=7.5 Hz, J=1.5 Hz, 1H), 6.75 (dd, J=7.5 Hz, J=1.5 Hz, 1H), 4.62 (br s, 1H), 3.40 (t, J=7.0 Hz, 2H), 2.60 (t, J=8.0 Hz, 2H), 1.85 (quintet, J=6.7 Hz, 2H), 1.62 (quintet, J=7.2 Hz, 2H), 1.4 (quintet, J=7.5 Hz, 2H), 1.40-1.35 (m, 4H).

5-Bromo-1-(4-hydroxy-phenyl)-pentane (9.4) was synthesized as in 9.1 using 8.4 (3.43 g, 12.7 mmol) in anhydrous CH₂Cl₂ (120 mL), and BBr₃ (1M solution in CH₂Cl₂, 32 mL, 32 mmol). The title compound (9.4) was isolated as a viscous liquid after purification by flash column chromatography (2.84 g, 92% yield).

¹H NMR (500 MHz, CDCl₃) δ 7.04 (d, J=8.7 Hz, 2H), 6.75 (d, J=8.7 Hz, 2H), 4.68 (br s, 1H), 3.34 (t, J=6.7 Hz, 2H), 2.55 (t, J=7.7 Hz, 2H), 1.88 (quintet, J=7.7 Hz, 2H), 1.60 (quintet, J=7.7 Hz, 2H), 1.46 (quintet, J=7.5 Hz, 2H).

### 7-Bromo-1-(4-benzyloxy-phenyl)-heptane (10.1)

To a stirred solution of 9.1 (0.9 g, 3.32 mmol) in anhydrous acetone (40 mL), was added anhydrous K₂CO₃ (1.38 g, 10 mmol) and benzyl bromide (0.624 g, 3.65 mmol) and the mixture was refluxed for 6 h. The reaction mixture was cooled to room temperature, diluted with acetone and solid materials were filtered off. The filtrate was evaporated under reduced pressure and the residue obtained was dissolved in diethyl ether (50 mL). The ethereal solution was washed with water and brine, dried (MgSO₄) and evaporated. Purification by flash column chromatography on silica gel (5% Et₂O-hexane) afforded 10.1 (0.938 g, 78% yield) as a white solid (m p 32-34° C.).

¹H NMR (500 MHz, CDCl₃) δ 7.43 (d, J=7.0 Hz, 2H), 7.38 (t, J=7.0 Hz, 2H), 7.32 (t, J=7.0 Hz 1H), 7.08 (d, J=8.7 Hz, 2H) 6.90 (d, J=8.7 Hz 2H), 5.04 (s, 2H), 3.34 (t, J=7.0 Hz, 2H), 2.54 (t, J=7.7 Hz, 2H), 1.85 (quintet, H=7.5 Hz, 2H), 1.58 (quintet, J=7.5 Hz, 2H), 1.46-1.38 (m, 2H), 1.37-1.30 (m, 4H).

7-Bromo-1-(3-benzyloxy-phenyl)-heptane (10.2) was prepared as in 10.1 using 9.2 (0.4 g, 1.48 mmol), K₂CO₃ (0.612 g, 4.44 mmol) and benzyl bromide (0.278 g, 1.63 mmol). The title compound (10.2) was isolated as a viscous liquid after purification by flash column chromatography (0.411 g, 77% yield).

¹H NMR (500 MHz, CDCl₃) δ 7.44 (d, J=7.5 Hz, 2H), 7.39 (t, J=7.5 Hz, 2H), 7.32 (t, J=7.5 Hz 1H), 7.19 (t, J=7.2 Hz, 1H) 6.83-6.77 (m, 3H), 5.05 (s, 2H), 3.40 (t, J=6.77 Hz, 2H), 2.56 (t, J=7.7 Hz, 2H), 1.84 (quintet, J=7.0 Hz, 2H), 1.60 (quintet, J=7.7 Hz, 2H), 1.42 (quintet, J=7.0 Hz, 2H), 1.35-1.32 (m, 4H).

7-Bromo-1-(2-benzyloxy-phenyl)-heptane (10.3) was prepared as in 10.1 using 9.3 (0.23 g, 0.85 mmol), K₂CO₃ (0.352 g, 2.55 mmol) and benzyl bromide (0.16 g, 0.935 mmol). The title compound (10.3) was isolated as a viscous liquid after purification by flash column chromatography (0.24 g, 78% yield).

¹H NMR (500 MHz, CDCl₃) δ 7.44 (d, J=7.5 Hz, 2H), 7.39 (t, J=7.5 Hz, 2H), 7.32 (t, J=7.5 Hz 1H), 7.18-7.13 (m, 2H), 6.92-6.88 (m, 2H), 5.08 (s, 2H), 3.37 (t, J=7.0 Hz, 2H), 2.67 (t, J=7.7 Hz, 2H), 1.82 (quintet, J=7.2 Hz, 2H), 1.62 (quintet, J=7.5 Hz, 2H), 1.39 (quintet, J=7.7 Hz, 2H), 1.36-1.32 (m, 4H).

5-Bromo-1-(4-benzyloxy-phenyl)-pentane (10.4) was prepared as in 10.1 using 9.4 (2.99 g, 12.3 mmol), K₂CO₃ (4.24 g, 30.75 mmol) and benzyl bromide (2.31 g, 13.53 mmol). The title compound (10.4) was isolated as a white semi-solid after purification by flash column chromatography (3.11 g, 76% yield).

¹H NMR (500 MHz, CDCl₃) δ 7.42 (d, J=7.5 Hz, 2H), 7.37 (t, J=7.5 Hz, 2H), 7.31 (t, J=7.5 Hz 1H), 7.08 (d, J=8.5 Hz, 2H), 6.90 (d, J=8.5 Hz, 2H), 5.03 (s, 2H), 3.39 (t, J=6.7 Hz, 2H), 2.56 (t, J=7.7 Hz, 2H), 1.87 (quintet, J=6.7 Hz, 2H), 1.61 (quintet J=7.7 Hz, 2H), 1.46 (quintet J=6.7 Hz, 2H).

### 7-(4-Benzyloxy-phenyl)-heptanesulfonic acid sodium salt (11.1)

A stirred mixture of 10.1 (0.9 g, 2.50 mmol) and anhydrous Na₂SO₃ (0.423 g, 3.36 mmol) in EtOH (20 mL)/H₂O (10 ml) was heated under reflux (6 h) or microwaved using a CEM-discover system (ram time: 2 min, hold time: 5 min, temperature: 150° C., pressure: 250 psi, power: 250 W). The reaction mixture was cooled to room temperature and the solvent evaporated under reduced pressure. The residue obtained was scrupulously dried under high vacuum and the crude product (10.1, pale yellow solid) was used in the next step without further purification.

7-(3-Benzyloxy-phenyl)-heptanesulfonic acid sodium salt (11.2). Following the procedure described for 11.1 using 10.2 (0.4 g, 1.1 mmol), Na₂SO₃ (0.19 g, 1.5 mmol) and EtOH (8 mL)/H₂O (4 ml) mixture, the crude 11.2 was obtained and used in the next step without further purification.

7-(2-Benzyloxy-phenyl)-heptanesulfonic acid Sodium salt (11.3). Following the procedure described for 11.1 using 10.3 (0.231 g, 0.64 mmol), Na₂SO₃ (0.11 g, 0.89 mmol) and EtOH (8 mL)/H₂O (4 ml) mixture, the crude 11.3 was obtained and used in the next step without further purification.

5-(4-Benzyloxy-phenyl)-pentanesulfonic acid Sodium salt (11.4). Following the procedure described for 11.1 using 10.4 (0.95 g, 2.85 mmol), Na₂SO₃ (0.50 g, 4.0 mmol) and EtOH (25 mL)/H₂O (7 ml) mixture, the crude 11.4 was obtained and used in the next step without further purification.

### 7-(4-Benzyloxy-phenyl)-heptanesulfonyl chloride (12.1)

To a stirred suspension of 11.1 (0.96 g, 2.50 mmol) in anhydrous benzene (20 mL)/DMF (2 ml), was added thionyl chloride (0.89 g, 7.5 mmol) and the resulting mixture was heated at 50° C. for 3 h under argon. The reaction mixture was quenched by dropwise addition of water (10 mL) at room temperature and extracted with diethyl ether. The organic layer was washed with brine, dried (MgSO₄) and the solvent was evaporated under reduced pressure. Purification by flash column chromatography on silica gel (20% diethyl ether-hexane) afforded 12.1 in 40% yield from 10.1 (0.38 g). White solid. m p 33-35° C.

¹H NMR (500 MHz, CDCl₃) δ 7.44 (d, J=7.5 Hz, 2H), 7.38 (t, J=7.5 Hz, 2H), 7.32 (t, J=7.5 Hz 1H), 7.08 (d, J=8.5 Hz, 2H), 6.90 (d, J=8.5 Hz, 2H), 5.04 (s, 2H), 3.64 (m as t, half of an AA′XX′ system, 2H), 2.55 (t, J=7.5 Hz, 2H), 2.03 (quintet, J=7.7 Hz, 2H), 1.62-1.54 (m, 2H), 1.52-1.46 (m, 2H), 1.40-1.30 (m, 4H).

7-(3-Benzyloxy-phenyl)-heptanesulfonyl chloride (12.2) was synthesized as described in 12.1 using 11.2 (0.42 g, 1.1 mmol) and thionyl chloride (0.36 g, 3 mmol) in benzene (9 mL)/DMF (1 mL). Purification by flash column chromatography on silica gel gave the title compound (0.163 g, 39% yield from 10.2) as a viscous liquid.

¹H NMR (500 MHz, CDCl₃) δ 7.44 (d, J=7.5 Hz, 2H), 7.39 (t, J=7.5 Hz, 2H), 7.32 (t, J=7.5 Hz, 1H), 7.19 (t, J=7.2 Hz, 1H), 6.82-6.77 (m, 3H), 5.05 (s, 2H), 3.64 (m as t, half of an AA′XX′ system, 2H), 2.58 (t, J=7.5 Hz, 2H), 2.02 (quintet, J=7.5 Hz, 2H), 1.62 (quintet, J=7.5 Hz, 2H), 1.48 (quintet, J=7.5 Hz, 2H), 1.42-1.32 (m, 4H).

7-(2-Benzyloxy-phenyl)-heptanesulfonyl chloride (12.3) was synthesized as described in 12.1 using 11.3 (0.46 g, 0.64 mmol) and thionyl chloride (0.228 g, 1.92 mmol) in benzene (9 mL)/DMF (1 mL). Purification by flash column chromatography on silica gel gave the title compound (0.092 g, 38% yield from 10.3) as a viscous liquid.

¹H NMR (500 MHz, CDCl₃) δ 7.44 (d, J=7.5 Hz, 2H), 7.39 (t, J=7.5 Hz, 2H), 7.33 (t, J=7.5 Hz 1H), 7.18-7.33 (m, 2H), 6.92-6.88 (m, 2H), 5.08 (s, 2H), 3.58 (m as t, half of an AA′XX′ system, 2H), 2.67 (t, J=7.7 Hz, 2H), 1.99 (quintet, J=7.5 Hz, 2H), 1.62 (quintet, J=7.5 Hz, 2H), 1.46-1.4 (m, 2H), 1.36-1.32 (m, 4H).

5-(4-Benzyloxy-phenyl)-pentanesulfonyl chloride (12.4) was synthesized as described in 12.1 using 11.4 (0.96 g, 2.85 mmol) and thionyl chloride (1.00 g, 8.55 mmol) in benzene (27 mL)/DMF (3 mL). Purification by flash column chromatography on silica gel gave the title compound (0.36 g, 37% yield from 10.4) as a white solid (m p 58-60° C.).

¹H NMR (500 MHz, CDCl₃) δ 7.43 (d, J=7.5 Hz, 2H), 7.38 (t, J=7.5 Hz, 2H), 7.32 (t, J=7.5 Hz 1H), 7.07 (d, J=8.7 Hz, 2H), 6.90 (d, J=8.7 Hz, 2H), 5.04 (s, 2H), 3.64 (m as t, half of an AA′XX′ system, 2H), 2.56 (t, J=7.2 Hz, 2H), 2.06 (quintet, J=7.7 Hz, 2H), 1.66 (quintet, J=7.5 Hz, 2H), 1.46 (quintet, J=7.7 Hz, 2H).

### 7-(4-Benzyloxy-phenyl)-heptanesulfonyl fluoride (13.1)

To a stirred solution of 12.1 (0.345 g, 0.9 mmol) in dry acetone (20 mL), was added anhydrous NH₄F (0.066 g, 1.8 mmol) and the mixture refluxed for 2 hours. The reaction mixture was cooled to room temperature, the solvent was evaporated, and the residue obtained was dissolved in diethyl ether (20 mL). The ethereal solution was successively washed with water and brine, dried (MgSO₄) and concentrated under reduced pressure. Purification by flash column chromatography on silica gel (20% diethyl ether hexane) afforded 13.1 (0.306 g, 93% yield) as a white solid (m p 35-38° C.).

¹H NMR (500 MHz, CDCl₃) δ 7.43 (d, J=7.5 Hz, 2H), 7.38 (t, J=7.5 Hz, 2H), 7.32 (t, J=7.5 Hz 1H), 7.08 (d, J=8.7 Hz, 2H), 6.90 (d, J=8.7 Hz, 2H), 5.04 (s, 2H), 3.36-3.32 (m, 2H), 2.54 (t, J=7.5 Hz, 2H), 1.94 (quintet, J=7.5 Hz, 2H), 1.62-1.54 (m, 2H), 1.52-1.44 (m, 2H), 1.40-1.30 (m, 4H).

7-(3-Benzyloxy-phenyl)-heptanesulfonyl fluoride (13.2) was prepared as in 13.1 using 12.2 (0.149 g, 0.39 mmol) and NH₄F (0.029 g, 0.78 mmol) in dry acetone (10 mL). Purification by flash column chromatography on silica gel gave the title compound (0.128 g, 91% yield) as a viscous liquid.

¹H NMR (500 MHz, CDCl₃) δ 7.43 (d, J=7.5 Hz, 2H), 7.39 (t, J=7.5 Hz, 2H), 7.32 (t, J=7.5 Hz, 1H), 7.19 (t, J=7.2 Hz, 1H), 6.82-6.77 (m, 3H), 5.05 (s, 2H), 3.36-3.32 (m, 2H), 2.58 (t, J=7.5 Hz, 2H), 1.93 (quintet, J=7.7 Hz, 2H), 1.61 (quintet, J=7.5 Hz, 2H), 1.48 (quintet, J=7.2 Hz, 2H), 1.42-1.32 (m, 4H).

7-(2-Benzyloxy-phenyl)-heptanesulfonyl fluoride (13.3) was prepared as in 13.1 using 12.3 (0.09 g, 0.236 mmol) and NH₄F (0.018 g, 0.486 mmol) in dry acetone (10 mL). Purification by flash column chromatography gave the title compound (0.079 g, 92% yield) as a viscous liquid.

¹H NMR (500 MHz, CDCl₃) δ 7.43 (d, J=7.2 Hz, 2H), 7.39 (t, J=7.2 Hz, 2H), 7.33 (t, J=7.2 Hz 1H), 7.17-7.14 (m, 2H), 6.92-6.89 (m, 2H), 5.08 (s, 2H), 3.35-3.32 (m, 2H), 2.67 (t, J=7.5 Hz, 2H), 1.89 (quintet, J=7.7 Hz, 2H), 1.62 (quintet, J=7.5 Hz, 2H), 1.46-1.4 (m, 2H), 1.36-1.32 (m, 4H).

5-(4-Benzyloxy-phenyl)-pentanesulfonyl fluoride (13.4) was synthesized as described in 13.1 using, 12.4 (0.3 g, 0.87 mmol) and NH₄F (0.06 g, 1.64 mmol) in dry acetone (40 mL). Purification by flash column chromatography on silica gel gave the title compound (0.266 g, 91% yield) as a white solid (m p 66-68° C.).

¹H NMR (500 MHz, CDCl₃) δ 7.43 (d, J=7.5 Hz, 2H), 7.38 (t, J=7.5 Hz, 2H), 7.32 (t, J=7.5 Hz 1H), 7.08 (d, J=8.0 Hz, 2H), 6.90 (d, J=8.0 Hz, 2H), 5.04 (s, 2H), 3.35-3.32 (m, 2H), 2.58 (t, J=7.5 Hz, 2H), 1.96 (quintet, J=7.7 Hz, 2H), 1.65 (quintet J=7.5 Hz, 2H), 1.50 (quintet, J=7.5 Hz, 2H).

### 7-(4-Hydroxy-phenyl)-heptanesulfonyl fluoride (14.1)

To a solution of 13.1 (0.182 g, 0.5 mmol) in ethanedithiol (10 mL), at room temperature, under an argon atmosphere was added BF₃.Et₂O (0.282 g, 2.0 mmol). The reaction mixture was stirred at room temperature for 1 hour and then diluted with diethyl ether (20 mL) and water (10 mL). The organic layer was separated and the aqueous phase extracted with diethyl ether. The combined organic layer was washed with brine, dried over MgSO₄ and concentrated under reduced pressure. The residue obtained was chromatographed through a column of silica gel eluting with 50% diethyl ether-hexane to give 14.1 (0.096 g, 70% yield) as a white solid (m p 47-51° C.).

¹H NMR (500 MHz, CDCl₃) δ 7.08 (d, J=9.0 Hz, 2H), 6.90 (d, J=9.0 Hz, 2H), 4.08 (br s, 2H), 3.36-3.32 (m, 2H), 2.55 (t, J=8.0 Hz, 2H), 1.98-1.90 (m, 2H), 1.62-1.54 (m, 2H), 1.52-1.44 (m, 2H) 1.38-1.34 (m, 4H).

7-(3-Hydroxy-phenyl)-heptanesulfonyl fluoride (14.2) was synthesized as described in 14.1 using 13.2 (0.1 g, 0.26 mmol) in ethanedithiol (5 mL) and BF₃.Et₂O (0.14 g, 1.0 mmol). Purification by flash column chromatography on silica gel gave 14.2 (0.049 g, 69% yield) as a viscous liquid.

¹H NMR (500 MHz, CDCl₃) δ 7.14 (t, J=7.5 Hz, 1H), 6.74 (d, J=7.5 Hz, 1H), 6.66-6.64 (m, 2H), 4.70 (br s 1H), 3.36-3.32 (m, 2H), 2.56 (t, J=7.7 Hz, 2H), 1.94 (quintet, J=7.7 Hz, 2H), 1.61 (quintet, J=7.5 Hz, 2H), 1.49 (quintet, J=7.2 Hz, 2H), 1.42-1.32 (m, 4H).

7-(2-Hydroxy-phenyl)-heptanesulfonyl fluoride (14.3) was synthesized as described in 14.1 using 13.3 (0.065 g, 0.17 mmol) in ethanedithiol (5 mL) and BF₃.Et₂O (0.092 g, 0.65 mmol). Purification by flash column chromatography gave 14.3 (0.033 g, 70% yield) as a viscous liquid.

¹H NMR (500 MHz, CDCl₃) δ 7.11-7.06 (m, 2H), 6.87 (dt, J=7.7 Hz, J=1.0 Hz, 1H), 6.75 (dd, J=7.7 Hz, J=1.0 Hz, 1H), 4.70 (br s, 1H), 3.35-3.32 (m, 2H), 2.61 (t, J=7.2 Hz, 2H), 1.94 (quintet, J=7.7 Hz, 2H), 1.66-1.58 (m, 2H), 1.52-1.46 (m, 2H), 1.42-1.34 (m, 4H).

5-(4-Hydroxy-phenyl)-pentanesulfonyl fluoride (14.4) was synthesized as described in 14.1 using, 13.4 (0.28 g, 0.83 mmol) in ethanedithiol (10 mL) and BF₃.Et₂O (0.47 g, 3.32 mmol). Purification by flash column chromatography on silica gel gave 14.4 (0.139 g, 68% yield) as a white solid (m p 32-35° C.).

¹H NMR (500 MHz, CDCl₃) δ 7.02 (d, J=8.2 Hz, 2H), 6.76 (d, J=8.2 Hz, 2H), 4.65 (br s, 1H), 3.36-3.32 (m, 2H), 2.58 (t, J=7.2 Hz, 2H), 1.96 (quintet, J=7.7 Hz, 2H), 1.64 (quintet, J=7.5 Hz, 2H), 1.50 (quintet, J=7.5 Hz, 2H).

Sulfonyl fluoride 17 (shown in Scheme 3) was synthesized by a method depicted in Scheme 3 starting from commercially available 4-phenoxybutyl bromide (5.2).

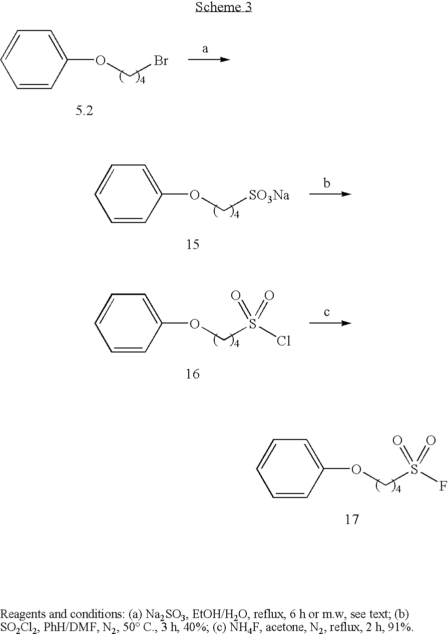

### Experimental Procedure

4-Phenoxybutyl sulfonic acid sodium salt (15). Following the procedure described for 11.1 using 5.2 (1.0 g, 4.37 mmol), Na₂SO₃ (0.77 g, 6.11 mmol), and EtOH (30 mL)/H₂O (10 mL) mixture, the crude 15 was obtained and used in the next step without further purification.

4-Phenoxybutyl sulfonyl chloride (16) was synthesized as described in 12.1 using 15 (1.0 g, 4.37 mmol) and thionyl chloride (1.55 g, 13.0 mmol) in benzene (40 mL)/DMF (4 mL). Purification by flash column chromatography on silica gel afforded 15 (0.434 g, 40% yield) as a white solid (m p 65-67° C.).

¹H NMR (500 MHz, CDCl₃) δ 7.29 (t, J=8.2 Hz, 2H), 6.97 (t, J=8.2 Hz, 1H), 6.89 (d, J=8.2 Hz, 2H), 4.04 (t, J=5.7 Hz, 2H), 3.80 (m as t, half of an AA′XX′ system, 2H), 2.29 (quintet, J=7.7 Hz, 2H), 2.01 (quintet, J=7.7 Hz, 2H).

4-Phenoxybutylsulfonyl fluoride (17) was synthesized as in 13.1 using 16 (0.4 g, 1.6 mmol) and NH₄F (0.118 g, 3.2 mmol) in dry acetone (20 mL). Purification by flash column chromatography on silica gel gave 17 (0.338 g, 91% yield) as a white solid (m p 74-76° C.).

¹H NMR (500 MHz, CDCl₃) δ 7.29 (t, J=7.5 Hz, 2H), 6.97 (t, J=7.5 Hz, 1H), 6.89 (d, J=7.5 Hz, 2H), 4.03 (t, J=5.5 Hz, 2H), 3.52-3.48 (m, 2H), 2.20 (quintet, J=7.7 Hz, 2H), 2.00 (quintet, J=8.0 Hz, 2H).

### 2. Synthesis of Sulfonyl Esters

Sulfonyl ester 18 (shown in Scheme 4) was synthesized by a method depicted in Scheme 4 starting from 12.1.

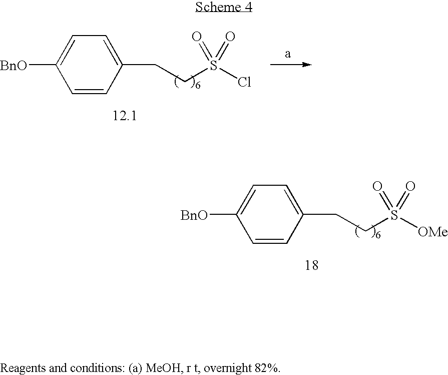

### Experimental Procedure

### 7-(4-Benzyloxy-phenyl)-heptane-1-sulfonic acid methyl ester (18)

A solution of 12.1 (0.050 g, 0.13 mmol) in MeOH (5 mL) was stirred at room temperature overnight.

The solvent was evaporated under reduced pressure and the residue obtained was dissolved in diethyl ether (20 mL). The ethereal solution was washed with water and brine, dried (MgSO₄) and evaporated under reduced pressure. Purification by flash column chromatography on silica gel (20% diethyl ether-hexane) gave the pure compound 18 (0.046 g, 82% yield), as a white solid (m p 57-59° C.).

¹H NMR. (500 MHz, CDCl₃) δ 7.43 (d, J=7.5 Hz, 2H), 7.38 (t, J=7.5 Hz, 2H), 7.32 (t, J=7.5 Hz 1H), 7.08 (d, J=8.7 Hz, 2H), 6.90 (d, J=8.7 Hz, 2H), 5.04 (s, 2H), 3.88 (s, 3H), 3.08 (m as t, half of an AA′XX′ system, J=7.7 Hz, 2H), 2.54 (t, J=7.7 Hz, 2H), 1.85 (quintet, J=7.7 Hz, 2H), 1.56 (quintet, J=7.0 Hz, 2H), 1.46-1.39 (m, 2H), 1.38-1.30 (m, 4H).

### 3. Synthesis of Trifluoromethyl Ketones

Trifluoromethyl ketones 23.1-12 and 24.1-10 (shown in Scheme 5) were synthesized by a method depicted in Scheme 5 starting from commercially available 2- or 3- or 4-(benzyloxy)phenol (19) and the appropriate ω-bromo-n-alkyl acid ethyl ester. 4-Phenoxy-butanoic acid (21.11) and 5-phenoxy-pentanoic acid (21.12) were also commercially available materials. Compound 24.5 was isolated in its hydrate form.

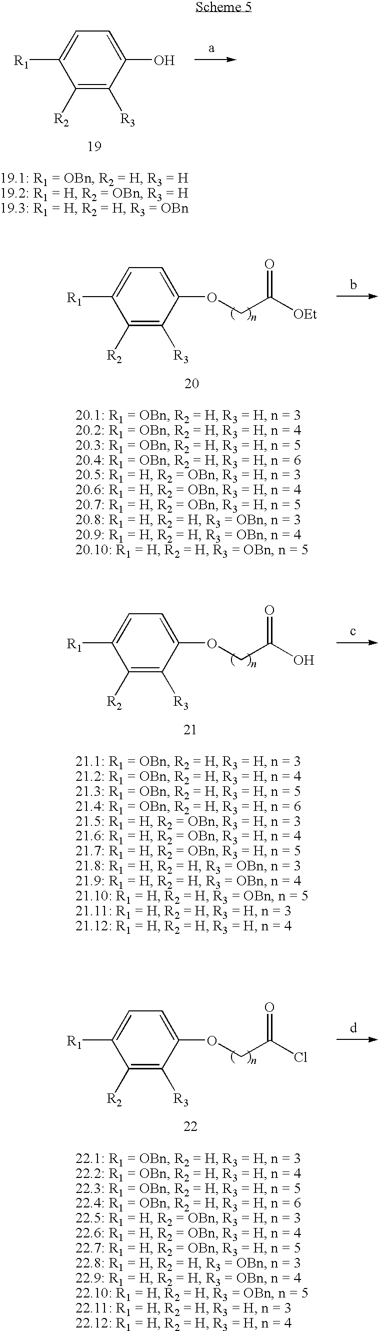

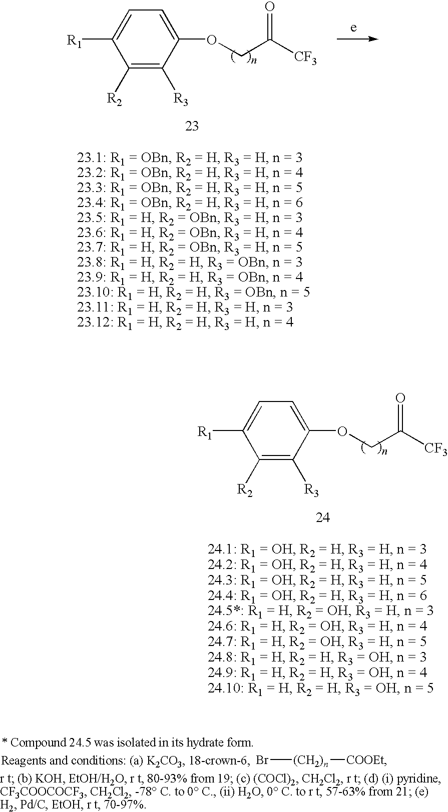

### Experimental Procedures

### Esters 20

A mixture of benzyloxyphenol (19) (1 equiv.), ω-bromo-n-alkyl acid ethyl ester (1.2 equiv.), potassium carbonate (1.2 equiv.), and 18-crown-6 (1 equiv.) in anhydrous acetonitrile was stirred overnight at room temperature under an argon atmosphere. The reaction mixture was evaporated and the residue was partitioned between water and diethyl ether. The organic phase was separated, washed with brine, dried (MgSO₄), and the solvent was removed under reduced pressure to leave the crude product (20). This product contains small amounts of unreacted ω-bromo-n-alkyl acid ethyl ester and it was used in the next step without purification. For analytical purposes 20.7 and 20.4 were further purified by flash column chromatography (20% diethyl ether-hexane) on silica gel. For a ¹H NMR spectrum and an alternative method for the preparation of 20.4 see experimental given for the synthesis of α-keto-heterocycles.

6-[3-(Benzyloxy)phenoxy]hexanoic acid ethyl ester (20.7). Colorless oil.

¹H NMR (500 MHz, CDCl₃) δ 7.41 (d, J=7.3 Hz, 2H), 7.36 (t, J=7.3 Hz, 2H), 7.30 (t, J=7.3 Hz, 1H), 7.14 (t, J=8.2 Hz, 1H), 6.57-6.52 (m, 2H), 6.49 (dd, J=8.2 Hz, J=1.8 Hz, 1H), 5.01 (s, 2H), 4.11 (q, J=7.2 Hz, 2H), 3.91 (t, J=6.5 Hz, 2H), 2.31 (t, J=7.5 Hz, 2H), 1.80-1.73 (m, 2H), 1.72-1.64 (m, 2H), 1.51-1.43 (m, 2H), 1.24 (t, J=7.2 Hz, 3H).

### Acids 21

A mixture of the crude ester (20) and potassium hydroxide (1.3 equiv.) in EtOH/H₂O (10:1 mixture) was heated under reflux for 3-4 hours. The reaction mixture was cooled to room temperature and the solvent was removed under reduced pressure. The residue obtained was dissolved in water, and the pH was adjusted to 1 using concentrated HCl solution. The precipitated crude acid was isolated by filtration and dissolved in ethyl acetate. The resulting solution was washed with brine, dried (MgSO₄), and the solvent was evaporated to give the product 21 in 80-93% yield (from 19).

### Selected Data of Synthesized Acids (21)

4-[4-(Benzyloxy)phenoxy]butanoic acid (21.1). White solid. m p 125-126° C.

¹H NMR (500 MHz, CDCl₃) δ 10.95 (br s, 1H), 7.41 (d, J=7.3 Hz, 2H), 7.37 (t, J=7.3 Hz, 2H), 7.31 (t, J=7.3 Hz, 1H), 6.90 (m as d, J=9.0 Hz, 2H), 6.81 (m as d, J=9.0 Hz, 2H), 5.01 (s, 2H), 3.97 (t, J=6.3 Hz, 2H), 2.58 (t, J=7.5 Hz, 2H), 2.09 (quintet, J=6.7 Hz, 2H); IR (neat) 2904, 2865, 1704, 1509 cm⁻¹.

5-[4-(Benzyloxy)phenoxy]pentanoic acid (21.2). White solid. m p 127-128° C.

¹H NMR (500 MHz, CDCl₃) δ 11.04 (br s, 1H), 7.42 (d, J=7.3 Hz, 2H), 7.37 (t, J=7.3 Hz, 2H), 7.31 (t, J=7.3 Hz, 1H), 6.89 (d, J=8.9 Hz, 2H), 6.81 (d, J=8.9 Hz, 2H), 5.01 (s, 2H), 3.92 (t, J=6.4 Hz, 2H), 2.44 (t, J=7.1 Hz, 2H), 1.85-1.79 (m, 4H); IR (neat) 2954, 2864, 1694, 1509 cm⁻¹.

6-[4-(Benzyloxy)phenoxy]hexanoic acid (21.3). White solid. m p 100-101° C.

¹H NMR (500 MHz, CDCl₃) δ 11.00 (br s, 1H), 7.42 (d, J=7.3 Hz, 2H), 7.37 (t, J=7.3 Hz, 2H), 7.31 (t, J=7.3 Hz, 1H), 6.89 (d, J=9.0 Hz, 2H), 6.81 (d, J=9.0 Hz, 2H), 5.01 (s, 2H), 3.90 (t, J=6.4 Hz, 2H), 2.39 (t, J=7.4 Hz, 2H), 1.78 (quintet, J=6.8 Hz, 2H), 1.71 (quintet, J=7.5 Hz, 2H), 1.60-1.45 (m, 2H); IR (neat) 2945, 2863, 1693, 1508 cm⁻¹.

7-[4-(Benzyloxy)phenoxy]heptanoic acid (21.4). White solid. m p 118-119° C.

¹H NMR (500 MHz, CDCl₃) δ 11.20 (br s, 1H), 7.42 (d, J=7.3 Hz, 2H), 7.37 (t, J=7.3 Hz, 2H), 7.31 (t, J=7.3 Hz, 1H), 6.89 (d, J=9.0 Hz, 2H), 6.81 (d, J=9.0 Hz, 2H), 5.01 (s, 2H), 3.89 (t, J=6.4 Hz, 2H), 2.36 (t, J=7.4 Hz, 2H), 1.79-1.72 (m, 2H), 1.70-1.63 (m, 2H), 1.51-1.37 (m, 4H).

4-[3-(Benzyloxy)phenoxy]butanoic acid (21.5). White solid. m p 76-77° C.

5-[3-(Benzyloxy)phenoxy]pentanoic acid (21.6). White solid. m p 71-72° C.

¹H NMR (500 MHz, CDCl₃) δ 10.82 (br s, 1H), 7.45 (d, J=7.3 Hz, 2H), 7.38 (t, J=7.3

Hz, 2H), 7.32 (t, J=7.3 Hz, 1H), 7.17 (t, J=8.2 Hz, 1H), 6.57 (dd, J=8.2 Hz, J=2.0 Hz, 1H), 6.54 (t, J=2.0 Hz, 1H), 6.50 (dd, J=8.2 Hz, J=2.0 Hz, 1H), 5.04 (s, 2H), 3.95 (t, J=5.7 Hz, 2H), 2.44 (t, J=6.7 Hz, 2H), 1.87-1.80 (m, 4H).

6-[3-(Benzyloxy)phenoxy]hexanoic acid (21.7). White solid. m p 72-73° C.

¹H NMR (500 MHz, CDCl₃) δ 11.31 (br s, 1H), 7.42 (d, J=7.3 Hz, 2H), 7.38 (t, J=7.3 Hz, 2H), 7.32 (t, J=7.3 Hz, 1H), 7.16 (t, J=8.2 Hz, 1H), 6.56 (dd, J=8.2 Hz, J=1.8 Hz, 1H), 6.54 (t, J=1.8 Hz, 1H), 6.50 (dd, J=8.2 Hz, J=1.8 Hz, 1H), 5.04 (s, 2H), 3.93 (t, J=6.5 Hz, 2H), 2.39 (t, J=7.5 Hz, 2H), 1.83-1.75 (m, 2H), 1.74-1.67 (m, 2H), 1.56-1.48 (m, 2H).

4-[2-(Benzyloxy)phenoxy]butanoic acid (21.8). White solid. m p 75-76° C.

1H NMR (500 MHz, CDCl₃) δ 9.50 (br s, 1H), 7.44 (d, J=7.4 Hz, 2H), 7.37 (t, J=7.4 Hz, 2H), 7.30 (t, J=7.4 Hz, 1H), 6.95-6.86 (m, 4H), 5.12 (s, 2H), 4.09 (t, J=5.9 Hz, 2H), 2.61 (t, J=7.1 Hz, 2H), 2.15 (quintet, J=6.5 Hz, 2H); IR (neat) 1693, 1590 cm⁻¹.

5-[2-(Benzyloxy)phenoxy]pentanoic acid (21.9). White solid. m p 74-75° C.

¹H NMR (500 MHz, CDCl₃) δ 11.02 (br s, 1H), 7.44 (d, J=7.4 Hz, 2H), 7.36 (t, J=7.4 Hz, 2H), 7.29 (t, J=7.4 Hz, 1H), 6.95-6.85 (m, 4H), 5.12 (s, 2H), 4.05 (t, J=5.9 Hz, 2H), 2.45 (t, J=7.1 Hz, 2H), 1.92-1.82 (m, 4H).

6-[2-(Benzyloxy)phenoxy]hexanoic acid (21.10). White solid. m p 77-78° C.

1H NMR (500 MHz, CDCl₃) δ 10.92 (br s, 1H), 7.44 (d, J=7.3 Hz, 2H), 7.36 (t, J=7.3 Hz, 2H), 7.29 (t, J=7.3 Hz, 1H), 6.95-6.84 (m, 4H), 5.12 (s, 2H), 4.03 (t, J=6.4 Hz, 2H), 2.36 (t, J=7.2 Hz, 2H), 1.85 (quintet, J=6.7 Hz, 2H), 1.71 (quintet, J=7.3 Hz, 2H), 1.59-1.51 (m, 2H).

### Carboxylic Acid Chlorides 22

To a solution of acid 21 (1 equiv.) in anhydrous CH₂Cl₂ at room temperature, under an argon atmosphere was added oxalyl chloride (2 equiv.) over a 2-min period. The mixture was stirred for 2 h, solvent and excess oxalyl chloride were removed under reduced pressure, and the crude carboxylic acid chloride (22) was used in the next step without further purification.

### Trifluoromethyl Ketones 23

To a solution of carboxylic acid chloride 22 in anhydrous CH₂Cl₂ at −78° C. under an argon atmosphere were added successively trifluoroacetic anhydride (6 equiv.) and dry pyridine (8 equiv.). The reaction mixture was stirred at −78° C. for 2 hours, and then it was allowed to warm to 0° C. and stirred for an additional 2 hours period. Water was added dropwise, the resulting mixture was warmed to room temperature, and extracted with CH₂Cl₂. The organic layer was washed with brine, dried (MgSO₄) and the solvent was evaporated. Following the workup, the crude mixture was chromatographed on a silica gel column (eluting with 30% diethyl ether-hexane), and the fraction that contains the product was concentrated and dried in high vacuum (in the presence of P₂O₅) to give compound 23 in 57-63% yield (from 21).

### Selected Data of Synthesized Trifluoromethyl Ketones (23)

1,1,1-Trifluoro-5-[4-(benzyloxy)phenoxy]-2-pentanone (23.1). White solid. m p 59-61° C.

¹H NMR (500 MHz, CDCl₃) δ 7.41 (d, J=7.3 Hz, 2H), 7.37 (t, J=7.3 Hz, 2H), 7.30 (t, J=7.3 Hz, 1H), 6.89 (m as d, J=9.0 Hz, 2H), 6.79 (m as d, J=9.0 Hz, 2H), 5.01 (s, 2H), 3.96 (t, J=5.7 Hz, 2H), 2.93 (t, J=7.0 Hz, 2H), 2.14 (quintet, J=6.5 Hz, 2H); IR (neat) 1765, 1509 cm⁻¹.

1,1,1-Trifluoro-6-[4-(Benzyloxy)phenoxy]-2-hexanone (23.2). White solid. m p 95.5-96° C.

1H NMR (500 MHz, CDCl₃) δ 7.42 (d, J=7.3 Hz, 2H), 7.38 (t, J=7.3 Hz, 2H), 7.31 (t, J=7.3 Hz, 1H), 6.90 (d, J=8.9 Hz, 2H), 6.81 (d, J=8.9 Hz, 2H), 5.01 (s, 2H), 3.93 (t, J=6.4 Hz, 2H), 2.82 (t, J=7.1 Hz, 2H), 1.88 (quintet, J=7.1 Hz, 2H), 1.81 (quintet, J=6.6 Hz, 2H); IR (neat) 1759, 1509 cm⁻¹.

1,1,1-Trifluoro-7-[4-(Benzyloxy)phenoxy]-2-heptanone (23.3). White solid. m p 59-60° C. IR (neat) 1761, 1509 cm⁻¹.

1,1,1-Trifluoro-8-[4-(Benzyloxy)phenoxy]-2-octanone (23.4). White solid. m p 82-83° C.

¹H NMR (500 MHz, CDCl₃) δ 7.42 (d, J=7.3 Hz, 2H), 7.38 (t, J=7.3 Hz, 2H), 7.31 (t, J=7.3 Hz, 1H), 6.89 (d, J=8.9 Hz, 2H), 6.81 (d, J=8.9 Hz, 2H), 5.01 (s, 2H), 3.90 (t, J=6.4 Hz, 2H), 2.73 (t, J=7.1 Hz, 2H), 1.80-1.67 (m, 4H), 1.52-1.45 (m, 2H), 1.44-1.36 (m, 2H).

1,1,1-Trifluoro-5-[3-(Benzyloxy)phenoxy]-2-pentanone (23.5). Colorless viscous oil.

¹H NMR (500 MHz, CDCl₃) δ 7.42 (d, J=7.3 Hz, 2H), 7.38 (t, J=7.3 Hz, 2H), 7.31 (t, J=7.3 Hz, 1H), 7.16 (t, J=8.2 Hz, 1H), 6.58 (dd, J=8.2 Hz, J=2.0 Hz, 1H), 6.52 (t, J=2.0 Hz, 1H), 6.48 (dd, J=8.2 Hz, J=2.0 Hz, 1H), 5.03 (s, 2H), 3.96 (t, J=5.9 Hz, 2H), 2.92 (t, J=6.9 Hz, 2H), 2.14 (quintet, J=6.5 Hz, 2H); IR (neat) 1763, 1591 cm⁻¹.

1,1,1-Trifluoro-6-[3-(Benzyloxy)phenoxy]-2-hexanone (23.6). Colorless viscous oil.

¹H NMR (500 MHz, CDCl₃) δ 7.42 (d, J=7.3 Hz, 2H), 7.38 (t, J=7.3 Hz, 2H), 7.32 (t, J=7.3 Hz, 1H), 7.17 (t, J=8.2 Hz, 1H), 6.58 (dd, J=8.2 Hz, J=2.0 Hz, 1H), 6.53 (t, J=2.0 Hz, 1H), 6.49 (dd, J=8.2 Hz, J=2.0 Hz, 1H), 5.04 (s, 2H), 3.96 (t, J=5.9 Hz, 2H), 2.81 (t, J=6.8 Hz, 2H), 1.91-1.78 (m, 4H).

1,1,1-Trifluoro-7-[3-(Benzyloxy)phenoxy]-2-heptanone (23.7). Colorless viscous oil.

1,1,1-Trifluoro-5-[2-(Benzyloxy)phenoxy]-2-pentanone (23.8). White solid. m p 50-51° C.

¹H NMR (500 MHz, CDCl₃) δ 7.42 (d, J=7.4 Hz, 2H), 7.36 (t, J=7.4 Hz, 2H), 7.30 (t, J=7.4 Hz, 1H), 6.96-6.89 (m, 4H), 5.09 (s, 2H), 4.06 (t, J=5.9 Hz, 2H), 2.98 (t, J=7.0 Hz, 2H), 2.16 (quintet, J=6.5 Hz, 2H); IR (neat) 1763, 1593 cm⁻¹.

1,1,1-Trifluoro-6-[2-(Benzyloxy)phenoxy]-2-hexanone (23.9). Colorless viscous oil.

¹H NMR (500 MHz, CDCl₃) δ 7.42 (d, J=7.4 Hz, 2H), 7.35 (t, J=7.4 Hz, 2H), 7.29 (t, J=7.4 Hz, 1H), 6.93 (d, J=7.4 Hz, 1H), 6.91-6.86 (m and t overlapping, especially 6.90, t, J=3.9 Hz, 3H), 5.10 (s, 2H), 4.04 (t, J=5.9 Hz, 2H), 2.80 (t, J=6.9 Hz, 2H), 1.93-1.82 (m, 4H).

1,1,1-Trifluoro-7-[2-(Benzyloxy)phenoxy]-2-heptanone (23.10). White solid. m p 31-32° C.

1H NMR (500 MHz, CDCl₃) δ 7.43 (d, J=7.3 Hz, 2H), 7.36 (t, J=7.3 Hz, 2H), 7.30 (t, J=7.3 Hz, 1H), 6.95-6.85 (m, 4H), 5.11 (s, 2H), 4.03 (t, J=6.4 Hz, 2H), 2.70 (t, J=7.1 Hz, 2H), 1.86 (quintet, J=6.7 Hz, 2H), 1.75 (quintet, J=7.3 Hz, 2H), 1.59-1.50 (m, 2H).

1,1,1-Trifluoro-5-phenoxy-2-pentanone (23.11). Colorless viscous oil.

¹H NMR (500 MHz, CDCl₃) δ 7.28 (t, J=7.4 Hz, 2H), 6.95 (t, J=7.4 Hz, 1H), 6.87 (d, J=7.4 Hz, 2H), 4.00 (t, J=5.8 Hz, 2H), 2.95 (t, J=7.0 Hz, 2H), 2.17 (quintet, J=6.4 Hz, 2H); ¹³C NMR (126 MHz, CDCl₃) δ 191.6 (q, J=35 Hz, C═O), 158.9, 129.9, 121.4, 116.0 (q, J=292 Hz, CF₃), 114.8, 66.1, 33.5, 22.8; IR (neat) 1763, 1601, 1588, 1498 cm⁻¹; mass spectrum m/z (relative intensity) 232 (M⁺, 25), 139 (24), 94 (100), 77 (16), 69 (27). Exact mass calculated for C₁₁H₁₁O₂F₃; 232.0711; found, 232.0714.

1,1,1-Trifluoro-6-phenoxy-2-hexanone (23.12). White solid. m p 50-51° C.

¹H NMR (500 MHz, CDCl₃) δ 7.28 (t, J=7.4 Hz, 2H), 6.94 (t, J=7.4 Hz, 1H), 6.88 (d, J=7.4 Hz, 2H), 3.98 (t, J=5.9 Hz, 2H), 2.83 (t, J=6.7 Hz, 2H), 1.95-1.80 (m, 4H); ¹³C NMR (126 MHz, CDCl₃) δ 191.7 (q, J=35 Hz, C═O), 159.2, 129.9, 121.2, 116.0 (q, J=291 Hz, CF₃), 114.8, 67.5, 36.4, 28.6, 19.8; IR (neat) 1759, 1601, 1585, 1500 cm⁻¹.

### Trifluoromethyl Ketones 24

To a solution of trifluoromethyl ketone 23 (1 equiv.) in EtOH was added 10% Pd/C (7% w/w), and the resulting suspension was stirred vigorously under hydrogen atmosphere, overnight at room temperature. The catalyst was removed by filtration through Celite, and the filtrate was evaporated under reduced pressure. The residue obtained was chromatographed on a silica gel column (eluting with 60% diethyl ether-hexane), and the fraction that contains the product was concentrated and dried in high vacuum (in the presence of P₂O₅) to give compound 24 in 70-97% yield. Especially in case of compound 24.5 the hydrate was isolated in 80% yield.

### Selected Data of Synthesized Trifluoromethyl Ketones (24)

1,1,1-Trifluoro-5-[4-(hydroxy)phenoxy]-2-pentanone (24.1). Colorless viscous oil.

1H NMR (500 MHz, CDCl₃) δ 6.76 (m as br s, 4H), 4.51 (br s, 1H), 3.95 (t, J=5.8 Hz, 2H), 2.95 (t, J=7.0 Hz, 2H), 2.15 (quintet, J=6.5 Hz, 2H); ¹³C NMR (126 MHz, CDCl₃) δ 191.5 (q, J=35 Hz, C═O), 152.7, 149.8, 116.2, 115.7, 115.6 (q, J=292 Hz, CF₃), 66.7, 33.2, 22.5; IR (neat) 3379 br, 1763, 1509 cm⁻¹.

1,1,1-Trifluoro-6-[4-(hydroxy)phenoxy]-2-hexanone (24.2). White solid. m p 63-64° C.

¹H NMR (500 MHz, CDCl₃) δ 6.76 (m as br s, 4H), 4.57 (br s, 1H), 3.92 (t, J=6.4 Hz, 2H), 2.82 (t, J=7.1 Hz, 2H), 1.88 (quintet, J=7.1 Hz, 2H), 1.81 (quintet, J=6.6 Hz, 2H); IR (neat) 3398 br, 1754, 1509 cm⁻¹.

1,1,1-Trifluoro-7-[4-(hydroxy)phenoxy]-2-heptanone (24.3). Colorless viscous oil.

IR (neat) 3386 br, 1762, 1509 cm⁻¹.

1,1,1-Trifluoro-8-[4-(hydroxy)phenoxy]-2-octanone (24.4). White solid. m p 61-62° C.

¹H NMR (500 MHz, CDCl₃) δ 6.77 (m as d, J=9.1 Hz, 2H), 6.75 (m as d, J=9.1 Hz, 2H), 4.40 (br s, 1H), 3.89 (t, J=6.4 Hz, 2H), 2.73 (t, J=7.1 Hz, 2H), 1.80-1.67 (m, 4H), 1.52-1.45 (m, 2H), 1.44-1.36 (m, 2H).

1,1,1-Trifluoro-2,2-dihydroxy-5-[3-(hydroxy)phenoxy]pentane (24.5). White solid. m p 76-77° C.

¹H NMR (500 MHz, CDCl₃/DMSO-d₆) δ 8.53 (br s, exchange with D₂₀, 1H), 7.06 (t, J=8.2 Hz, 1H), 6.47-6.42 (m, 2H), 6.39 (dd, J=8.2 Hz, J=1.9 Hz, 1H), 5.49 (br s, exchange with D₂O, 2H), 3.99 (t, J=6.1 Hz, 2H), 2.05 (m, 2H), 1.95 (t, J=7.1 Hz, 2H); IR (neat) 3300 br, 1605 cm⁻¹.

1,1,1-Trifluoro-6-[3-(hydroxy)phenoxy]-2-hexanone (24.6). Colorless viscous oil.

¹H NMR (500 MHz, CDCl₃) δ 7.11 (t, J=8.2 Hz, 1H), 6.46 (dd, J=8.2 Hz, J=2.2 Hz, 1H), 6.42 (dd, J=8.2 Hz, J=2.2 Hz, 1H), 6.39 (t, J=2.2 Hz, 1H), 5.19 (br s, 1H), 3.94 (t, J=5.9 Hz, 2H), 2.81 (t, J=6.8 Hz, 2H), 1.90-1.77 (m, 4H).

1,1,1-Trifluoro-7-[3-(hydroxy)phenoxy]-2-heptanone (24.7). Orange viscous oil.

1,1,1-Trifluoro-5-[2-(hydroxy)phenoxy]-2-pentanone (24.8). Colorless viscous oil.

¹H NMR (500 MHz, CDCl₃) δ 6.95 (d, J=7.7 Hz, 1H), 6.89 (m as quintet, J=3.9 Hz, 1H), 6.83 (d, J=4.2 Hz, 2H), 5.52 (br s, 1H), 4.11 (t, J=6.0 Hz, 2H), 2.96 (t, J=6.9 Hz, 2H), 2.23 (quintet, J=6.5 Hz, 2H).

1,1,1-Trifluoro-6-[2-(hydroxy)phenoxy]-2-hexanone (24.9). White solid. m p 51-52° C.

¹H NMR (500 MHz, CDCl₃) δ 6.94 (d, J=7.7 Hz, 1H), 6.90-6.86 (m, 1H), 6.85-6.82 (m, 2H), 5.60 (br s, 1H), 4.07 (t, J=5.7 Hz, 2H), 2.83 (t, J=6.3 Hz, 2H), 1.94-1.84 (m, 4H).

1,1,1-Trifluoro-7-[2-(hydroxy)phenoxy]-2-heptanone (24.10). White semi-solid.

¹H NMR (500 MHz, CDCl₃) δ 6.84 (d, J=7.3 Hz, 1H), 6.80-6.72 (m, 3H), 5.58 (br s, 1H), 3.95 (t, J=6.4 Hz, 2H), 2.66 (t, J=7.1 Hz, 2H), 1.75 (quintet, J=6.7 Hz, 2H), 1.66 (quintet, J=7.3 Hz, 2H), 1.46-1.38 (m, 2H).

Trifluoromethyl ketones 27.1-4 (shown in Scheme 6) were synthesized by a method depicted in Scheme 6. 4-Phenyl-butyric acid (25.1), 5-phenyl-pentanoic acid (25.2), 6-phenyl-hexanoic acid (25.3) and 5-(4-methoxy-phenyl)-pentanoic acid (25.4) were commercially available starting materials.

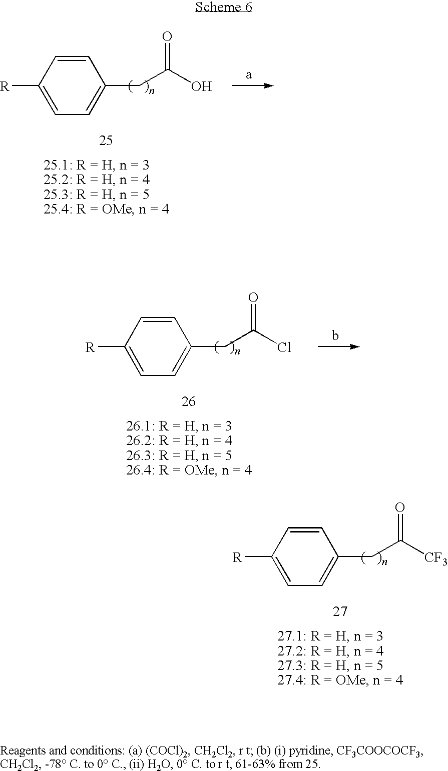

### Experimental Procedures

The synthesis of compounds 27 was carried out analogous to the preparation of compounds 23.

### Selected Data of Synthesized Analogs 27.

1,1,1-Trifluoro-5-phenyl-2-pentanone (27.1). Colorless viscous oil.

IR (neat) 1762, 1604, 1498, 1454, 1403 cm⁻¹.

1,1,1-Trifluoro-6-phenyl-2-hexanone (27.2). Colorless viscous oil.

¹H NMR (500 MHz, CDCl₃) δ 7.27 (t, J=7.5 Hz, 2H), 7.17 (t, J=7.5 Hz, 1H), 7.15 (d, J=7.5 Hz, 2H), 2.70 (t, J=7.2 Hz, 2H), 2.63 (t, J=7.7 Hz, 2H), 1.76-1.62 (m, 4H); ¹³C NMR (126 MHz, CDCl₃) δ 191.7 (q, J=35 Hz, C=0), 142.0, 128.8, 128.7, 126.3, 116.0 (q, J=292 Hz, CF₃), 36.6, 35.8, 30.8, 22.4; IR (neat) 1763, 1604, 1497, 1454, 1404 cm⁻¹.

1,1,1-Trifluoro-7-phenyl-2-heptanone (27.3). Colorless viscous oil.

¹H NMR (500 MHz, CDCl₃) δ 7.27 (t, J=7.5 Hz, 2H), 7.18 (t, J=7.5 Hz, 1H), 7.16 (d, J=7.5 Hz, 2H), 2.69 (t, J=7.2 Hz, 2H), 2.61 (t, J=7.7 Hz, 2H), 1.70 (quintet, J=7.6 Hz, 2H), 1.64 (quintet, J=7.6 Hz, 2H), 1.37 (quintet, J=7.7 Hz, 2H); IR (neat) 1763, 1604, 1497, 1454, 1402 cm⁻¹; mass spectrum m/z (relative intensity) 244 (M⁺, 21), 175 (8), 117 (20), 91 (100), 77 (6). Exact mass calculated for C₁₃H₁₅OF₃; 244.1075; found, 244.1073.

1,1,1-Trifluoro-6-(4-methoxy-phenyl)-2-hexanone (27.4). Colorless viscous oil.

¹H NMR (500 MHz, CDCl₃) δ 7.07 (d, J=8.4 Hz, 2H), 6.82 (d, J=8.4 Hz, 2H), 3.77 (s, 3H), 2.71 (t, J=6.9 Hz, 2H), 2.58 (t, J=7.4 Hz, 2H), 1.70 (quintet, J=7.1 Hz, 2H), 1.62 (quintet, J=6.8 Hz, 2H); IR (neat) 1763, 1612, 1584, 1512 cm⁻¹.

Trifluoromethyl ketones 30 and 35 (shown in Scheme 7) were synthesized by a method depicted in Scheme 7. 3-(Methoxycarbonyl)phenylboronic acid, 3-benzyloxyphenylboronic acid and 3-benzyloxybromobenzene (28) were commercially available starting materials while (3-bromophenyl)acetic acid methyl ester (31) was synthesized from commercially available 3-bromophenylacetic acid following a method disclosed in Luning, U et al., Eur. J. Org. Chem., 2002, 3294-3303.

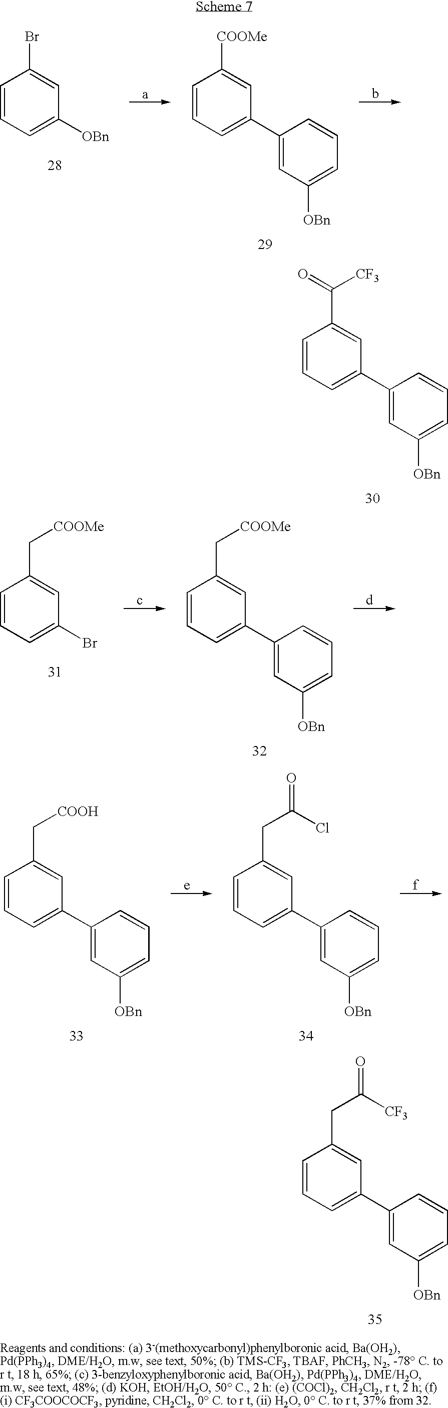

### Experimental Procedures

### 3′-Benzyloxy-biphenyl-3-carboxylic acid methyl ester (29)

A degassed mixture of 3-benzyloxy-phenyl bromide (28) (0.176 g, 0.67 mmol), 3-methoxycarbonylphenylboronic acid (0.18 g, 1 mmol), barium hydroxide (0.25 g, 1.47 mmol), Pd(PPh₃)₄ (0.077 g, 0.067 mmol), DME (5 mL) and H₂O (3 mL) was microwaved with vigorous stirring using a CEM-discover system (ram time: 2 min, hold time: 5 min, temperature: 120° C., pressure: 200 psi, power: 250 W). The crude reaction mixture filtered through a plug of celite and concentrated in vacuo. The residue obtained was purified by flash column chromatography (25% diethyl ether-hexane) to give the title compound (29) (0.118 g, 60% yield) as a viscous liquid.

¹H NMR (500 MHz, CDCl₃) δ 8.27 (t, J=1.5 Hz, 1H), 8.20 (dd, J=8.0 Hz, J=1.5 Hz, 1H), 7.76 (dd, J=8.0 Hz, J=2.0 Hz, 1H), 7.50 (t, J=8.0 Hz, 1H), 7.47 (d, J=7.5 Hz, 2H), 7.42-7.32 (m, 4H), 7.25-7.22 (m, 2H), 7.00 (dd, J=8.2 Hz, J=2.0 Hz, 1H), 5.13 (s, 2H), 3.95 (s, 3H).

### 1,1,1-Trifluoro-2-(3-benzyloxy-biphenyl-3-yl)-2-ethanone (30)

A solution of 29 (0.1 g, 0.314 mmol) in anhydrous toluene (5 mL) was cooled to −78° C., under nitrogen, and trifluoromethylrimethylsilane (62.5 mg, 0.44 mmol) was added. The mixture was stirred for 15 min at −78° C., then a 1M anhydrous solution of tetrabutylammonium fluoride in THF (0.026 ml, 0.026 mmol) was added and the resultant mixture was gradually warmed to room temperature. After stirring for 12 h at room temperature, the reaction mixture was diluted with 4N HCl solution (2 mL) and stirred for an additional 2 h period. The organic layer was separated and the aqueous layer was extracted with diethyl ether (20 mL). The combined organic layer was washed with aqueous saturated NaHCO₃ solution (5 mL) and brine, dried (MgSO₄) and concentrated under reduced pressure. The residue was purified by flash column chromatography on silica gel (25% diethyl ether-hexane) and the fraction that contains the product 30 and its hydrate form (2:1 ratio by ¹H NMR) was concentrated and dried in high vacuum (in the presence of P₂O₅) to give pure compound 30 (0.0876 g, 76% yield) as a viscous liquid.

¹H NMR (500 MHz, CDCl₃) δ 8.26 (s, 1H), 8.04 (d, J=7.5 Hz, 1H), 7.90 (d, J=8.0 Hz, 1H), 7.61 (t, J=7.5 Hz, 1H), 7.47 (d, J=8.0 Hz, 2H), 7.44-7.38 (m, 3H), 7.3 (t, J=7.2 Hz, 1H), 7.22-7.20 (m, 2H), 7.03 (dd, J=8.0 Hz, J=2.5 Hz, 1H), 5.08 (s, 2H).

2-(3-Benzyloxy-biphenyl-3-yl)-acetic acid methyl ester (32) was synthesized following the procedure described for the preparation of 29 using 3-bromo-phenyl acetic acid methyl ester (31) (0.31 g, 1.35 mmol), 3-benzyloxy-phenyl boronic acid (0.45 g, 2 mmol), barium hydroxide (0.5 g, 3 mmol) and Pd(PPh₃)₄ (0.15 g, 0.13 mmol), in DME (10 mL)/water (4 mL). Purification by flash column chromatography on silica gel gave pure compound 32 (0.22 g, 49% yield) as a white solid (m p 50-52° C.).

¹H NMR (500 MHz, CDCl₃) δ 7.49-7.45 (m, 4H), 7.42-7.32 (m, 5H), 7.27 (d, J=7.0 Hz, 1H), 7.21 (t, J=2.5 Hz, 1H) 7.19 ((dd, J=7.5 Hz, J=1.0 Hz, 1H), 6.97 (dd, J=8.0 Hz, J=2.5 Hz 1H), 5.1 (s, 2H), 3.71 (s, 3H), 3.69 (s, 2H).

### 2-(3-Benzyloxy-biphenyl-3-yl)-acetic acid (33)

A mixture of 26 (0.1 g, 0.3 mmol) and potassium hydroxide (0.08 g, 1.2 mmol) in wet EtOH (5 mL) was heated at 50° C., under nitrogen for 2 hours. The reaction mixture was cooled to room temperature and the solvent evaporated under reduced pressure. The residue obtained was dissolved in water (5 mL) and the pH was adjusted to 1 using 5% aqueous HCl solution (2 mL). The precipitated crude acid was isolated by filtration and dissolved in ethyl acetate. The resulting solution was washed with brine, dried (MgSO₄) and concentrated under reduced pressure to give 33 as a white solid (0.087 g, 91%), which was used in the next step without further purification.

### 1,1,1-Trifluoro-3-(3-benzyloxy-biphenyl-3-yl)-2-propanone (35)

To a solution of acid 33 (0.08 g, 0.25 mmol) in anhydrous CH₂Cl₂ at room temperature, under nitrogen, was added oxalyl chloride (0.25 mL, 0.5 mmol) over a 2-min period. The mixture was stirred for 2 hours, solvent and excess oxalyl chloride were removed under reduced pressure, and the crude carboxylic acid chloride (34) was used in the next step without further purification.

To a solution of 34 in anhydrous CH₂Cl₂ at 0° C. under a nitrogen atmosphere were added successively trifluoroacetic anhydride (1 mL, 1.5 mmol) and dry pyridine (0.16 mmol, 0.16 mL). The reaction mixture was stirred at 0° C. for 10 min, and then it was allowed to warm to room temperature and stirred for an additional 2 hours period. Water was added dropwise at 0° C., the resulting mixture was warmed to room temperature, and extracted with CH₂Cl₂. The organic layer was washed with dilute aqueous HCl solution, and saturated aqueous NaHCO₃ solution, dried (MgSO₄) and the solvent was evaporated. Following the workup, the crude mixture was chromatographed on a silica gel column (eluting with 30% diethyl ether-hexane) to give compound 35 (0.033 g, 36% yield) as a viscous liquid.

¹H NMR (500 MHz, CDCl₃) δ 7.56 (d, J=8.0 Hz, 1H), 7.49 (d, J=7.0 Hz, 2H), 7.47-7.41 (m, 4H), 7.40-7.35 (m, 3H), 7.23-7.19 (m, 3H), 7.03 (dd, J=8.0 Hz, J=2.5 Hz, 1H), 5.15 (s, 2H), 4.01 (s, 2H).

### 4. Synthesis of Carbamates

The carbamates 41.1, 41.2 or 41.3 shown in Scheme 8 were synthesized by a method depicted in Scheme 8 starting from commercially available 4-(4-methoxyphenyl)butanol (36).

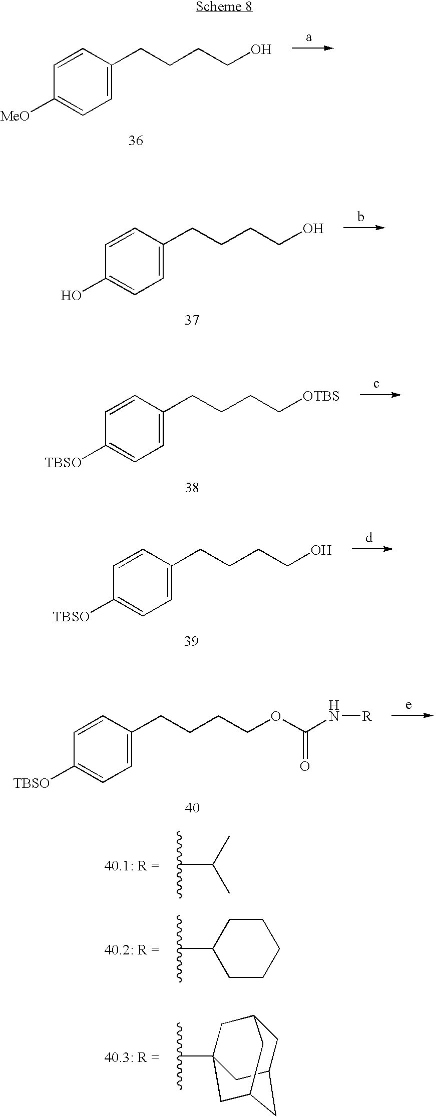

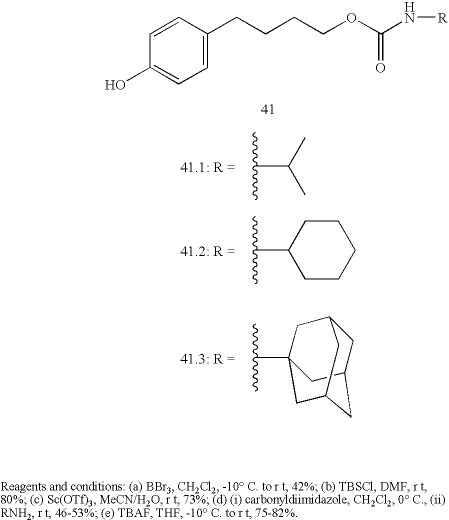

### Experimental Procedures

### 4-(4-Hydroxyphenyl)butanol (37)

To a stirred solution of 4-(4-methoxyphenyl)butanol (1 equiv.) in dry dichloromethane at −10° C. under an argon atmosphere was added boron tribromide (2.7 equiv., using a 1 M solution of boron tribromide in CH₂Cl₂). Stirring was continued at that temperature until completion of the reaction (4 hours). Unreacted boron tribromide was destroyed by addition of aqueous saturated NaHCO₃ solution at 0° C. The resulting mixture diluted with CH₂Cl₂ and water, the organic phase was separated, washed with brine, dried (MgSO₄) and evaporated. Purification by flash column chromatography on silica gel (30% diethyl ether-hexane) afforded the title compound in 42% yield.

### 1-(tert-Butyldimethylsilyloxy)-4-(tert-butyldimethylsilyloxybutyl)-benzene (38)

To a solution of imidazole (4 equiv.) in DMF was added 4-(4-hydroxyphenyl)butanol (1 equiv.) in DMF followed by tert-butyldimethylsilyl chloride (3 equiv.) in DMF. The reaction was allowed to stir at room temperature for 15 hours and then quenched by addition of saturated aqueous NaHCO₃ solution. The resulting mixture was extracted with diethyl ether, the ethereal extract was washed with water and brine, and dried over MgSO₄. Solvent evaporation and purification by flash column chromatography on silica gel (3% diethyl ether-hexane) afforded the title compound in 80% yield.

### 4-(4-tert-Butyldimethylsilyloxy)butanol (39)

To a solution of 1-(tert-butyldimethylsilyloxy)-4-(tert-butyldimethylsilyloxybutyl)-benzene (1 equiv.) in a mixture of acetonitrile/water (1:2.5) at room temperature was added scandium triflate (0.05 equiv.). The reaction mixture was stirred for 1 hour, diluted by addition of CH₂Cl₂ and the organic phase was separated. The aqueous phase was extracted with CH₂Cl₂ and the combined organic layer washed with brine, dried (MgSO₄) and evaporated. Purification by flash column chromatography on silica gel (20% diethyl ether-hexane) gave the title compound in 73% yield.

### Intermediate Carbamates (40)

To a suspension of carbonyldiimidazole (1.5 equiv.) in anhydrous dichloromethane at 0° C. was added 4-(4-tert-butyldimethylsilyloxy)butanol (1 equiv.) in dichloromethane. The reaction mixture was stirred at room temperature for 1 hour and then the appropriate amine (1.1 equiv.) was added. Stirring was continued until completion of the reaction (8-10 hours). The reaction mixture was diluted with diethyl ether and 10% aqueous HCl solution. The organic phase was separated, washed with brine, dried (MgSO₄) and evaporated. Purification by flash column chromatography on silica gel (10% diethyl ether-hexane) gave intermediate carbamate 40 in 46-53% yield.

### Carbamates (41)

To a stirred solution of intermediate carbamate 40 (1 equiv.) in THF at −10° C. was added dropwise tetra-n-butylammonium fluoride hydrate (1.3 equiv.) in THF. The reaction mixture was allowed to warm to room temperature, stirred for 1 hour and diluted with diethyl ether. The organic phase was separated, washed with water and brine, dried (MgSO₄) and evaporated. Purification by flash column chromatography on silica gel gave carbamate 41 in 75-82% yield.

### Selected data of synthesized carbamates (41)

4-(4-Hydroxyphenyl)butanol isopropylcarbamate (41.1). ¹H NMR (400 MHz, CDCl₃) δ 7.01 (d, J=8.4 Hz, 2H), 6.76 (d, J=8.4 Hz, 2H), 4.55 (br s, 1H), 4.06 (t as br s, 2H), 3.81 (m, 1H), 2.54 (t, J=5.8 Hz, 2H), 1.71-1.59 (m, 4H), 1.14 (d, J=6.5 Hz, 6H).

4-(4-Hydroxyphenyl)butanol cyclohexylcarbamate (41.2). ¹H NMR (400 MHz, CDCl₃) δ 6.99 (d, J=8.3 Hz, 2H), 6.75 (d, J=8.3 Hz, 2H), 6.23 (br s, 1H), 4.51 (br s, 1H), 4.05 (t as br s, 2H), 3.48 (m, 1H), 2.55 (t, J=5.8 Hz, 2H), 1.97-1.85 (m, 2H), 1.75-1.05 (m, 12H).

### 5. Synthesis of Ureas

Ureas 43.1 and 43.1 (shown in Scheme 9) were synthesized by a method depicted in Scheme 9 starting from commercially available 3-phenyl-propyl isocyanate (42) and 2-aminomethyl-pyridine or 2-aminopyridine.

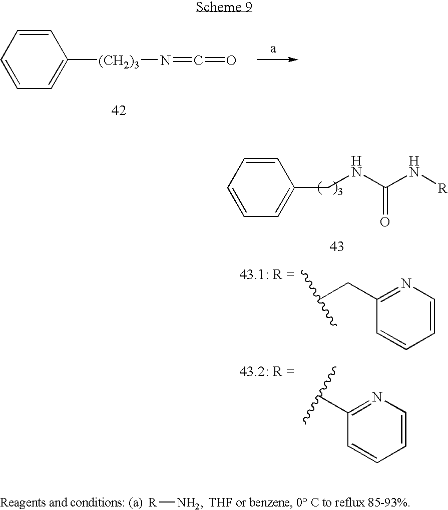

### Experimental Procedures

### N-(3-phenylpropyl)-N′-(2-pyridinylmethyl)-urea (43.1)

To a solution of 3-phenylpropyl isocyanate (1.8 mmol) in anhydrous THF (10 mL) at 0° C. under an argon atmosphere was added 2-aminomethyl-pyridine (1.8 mmol). The reaction mixture was stirred at 0° C. for 10 min, the solvent was evaporated under reduced pressure, and the resultant solid was recrystallized from CH₂Cl₂/Et₂O to give pure 43.1 in 92% yield. White solid. m p 89-90° C. When anhydrous benzene was used as solvent the product was directly crystallized out and isolated by filtration (93% yield).

¹H NMR (500 MHz, CDCl₃) δ 8.47 (d, J=4.4 Hz, 1H), 7.61 (td, J=7.6 Hz, J=1.1 Hz, 1H), 7.28-7.22 (m, 3H), 7.19-7.10 (m, 4H), 5.97 (t, J=4.9 Hz, 1H, NH), 5.30 (br s, 1H, NH), 4.45 (d, J=5.4 Hz, 2H), 3.20 (td as q, J=6.4 Hz, 2H), 2.61 (t, J=7.6 Hz, 2H), 1.79 (quintet, J=7.3 Hz, 2H); IR (neat), 3320, 3028, 2941, 2860, 1620, 1594, 1568, cm⁻¹.

### N-(3-phenylpropyl)-N′-(2-pyridinyl)-urea (43.2)

To a stirred solution of 3-phenylpropyl isocyanate (2 mmol) in anhydrous THF (15 mL) at 0° C. under an argon atmosphere was added 2-amino-pyridine (2 mmol). Following the addition, the reaction mixture was heated under reflux for 2 h, the solvent was evaporated under reduced pressure, and the resultant solid was recrystallized from CH₂Cl₂/Et₂O to give pure 43.2 in 85% yield. White solid. m p 127-128° C.

¹H NMR (500 MHz, CDCl₃) δ 9.72 and 9.66 (s and br s, overlapping, 2H, NH), 8.16 (d, J=4.3 Hz, 1H), 7.58 (t, J=7.1 Hz, 1H), 7.30 (t, J=7.4 Hz, 2H), 7.24 (d, J=7.4 Hz, 2H), 7.21 (t, J=7.4 Hz, 1H), 6.94 (d, J=7.1 Hz, 1H), 6.87 (m as t, J=6.4 Hz, 1H), 3.44 (td as q, J=6.4 Hz, 2H), 2.75 (t, J=7.6 Hz, 2H), 1.97 (quintet, J=7.2 Hz, 2H); IR (neat) 3221, 3054, 2980, 2918, 1682, 1602, 1583, 1549, 1480 cm⁻¹.

### 6. Synthesis of α-keto-heterocycles

α-Keto-oxadiazoles 49.1, 49.2 and 50 (shown in Scheme 10) were synthesized by a method depicted in Scheme 10 starting from 44.1 or 44.2 and 2-methyl-oxadiazole (47). Phenol (44.1) and 4-benzyloxy-phenol (44.2) were commercially available while 2-methyl-oxadiazole (47) was prepared by a method disclosed in Ainsworth, C et al., J. Org. Chem. Soc., 1966, 31, 3442-3444 and in Ohmoto, K et al., J. Med. Chem., 2001, 44, 1268-1285.

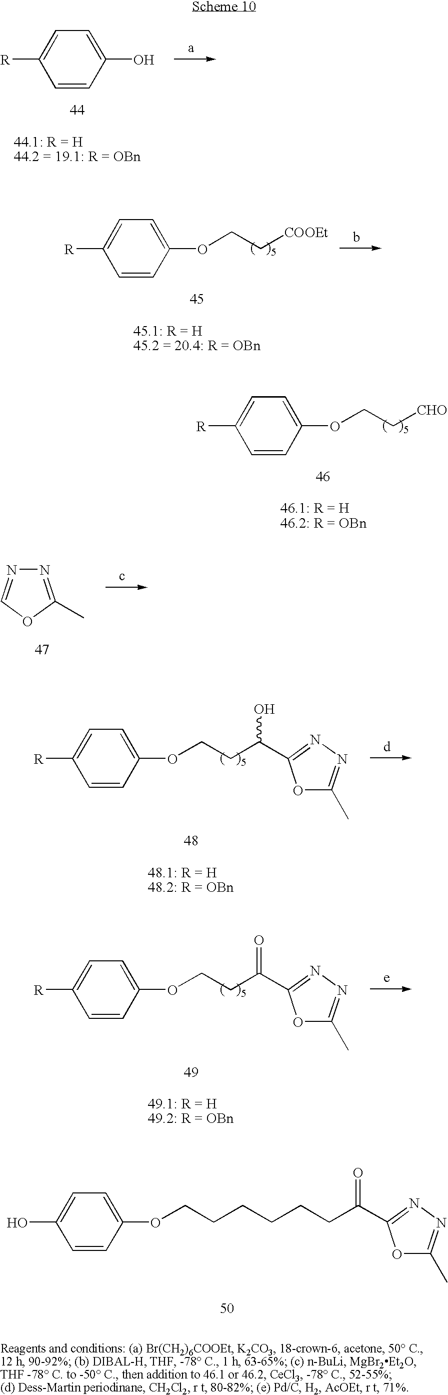

### Experimental Procedures

### 7-(Phenoxy)heptanoic acid ethyl ester (45.1)

To a solution of 44.1 (0.7 g, 7.5 mmol) in dry acetone (50 mL), under a nitrogen atmosphere, was added 18-crown-6 (1.584 g, 6 mmol), anhydrous potassium carbonate (2.07 g, 15 mmol), and ethyl 7-bromoheptanoate (1.18 g, 5 mmol) successively. The mixture was stirred at 50° C. overnight and then it was cooled to room temperature and the solvent removed in vacuo. The residue obtained was partitioned between diethyl ether (50 mL), and water (10 mL). The organic phase was separated and the aqueous layer extracted with diethyl ether. The combined organic layer was washed with brine, dried (MgSO₄) and the solvent was removed under reduced pressure. Purification by flash column chromatography (20% diethyl ether-hexane) afforded 45.1 (1.72 g, 92% yield) as a colorless liquid.

¹H NMR (500 MHz, CDCl₃) δ 7.27 (dt, J=7.7 Hz, J=1.5 Hz, 2H), 6.92 (dt, J=7.7 Hz, J=1.5 Hz 1H), 6.89 (d, J=7.7 Hz, 2H), 4.12 (q, J=7.0 Hz, 2H), 3.95 (t, J=6.2 Hz, 2H), 2.31 (t, J=7.7 Hz, 2H), 1.78 (quintet, J=6.5 Hz, 2H), 1.66 (quintet, J=7.5 Hz, 2H), 1.49 (quintet, J=7.2 Hz, 2H), 1.40 (quintet, J=8.2, 2H), 1.25 (t, J=7.0 Hz, 2H).

7-[4-(Benzyloxy)phenoxy]heptanoic acid ethyl ester (45.2/20.4). An alternative method for the synthesis of the title compound was carried out analogous to the preparation of 45.1 using 44.2 (0.45 g, 2.255 mmol), 18-crown-6 (1.056 g, 4 mmol), potassium carbonate (1.38 g, 10 mmol), and Br(CH₂)₆COOEt, (0.8 g, 3.37 mmol) in dry acetone (40 mL). Purification by flash column chromatography on silica gel (20% diethyl ether-hexane) gave 45.2/20.4 (1.08 g, 90% yield) as a white solid (m p 57-61° C.).

¹H NMR (500 MHz, CDCl₃) δ 7.42 (d, J=7.5 Hz, 2H), 7.37 (t, J=7.5 Hz, 2H), 7.31 (t, J=7.5 Hz, 1H), 6.89 (d, J=8.7 Hz, 2H), 6.82 (d, J=8.7 Hz, 2H), 5.01 (s, 2H), 4.12 (q, J=7.0 Hz, 2H), 3.89 (t, J=6.5 Hz, 2H), 2.30 (t, J=7.5 Hz, 2H), 1.76 (quintet, J=6.7 Hz, 2H), 1.66 (quintet, J=7.5 Hz, 2H), 1.47 (quintet, J=7.2 Hz, 2H), 1.38 (quintet, J=6.7 Hz, 2H), 1.25 (t, J=7.0 Hz, 2H).

### 7-(Phenoxy)heptanal (46.1)

To a stirred solution of 45.1 (0.56 g, 2.24 mmol) in dry THF (20 mL), at −78° C., under a nitrogen atmosphere was added diisobutylaluminum hydride (5 mL, 5 mmol, using a 1M solution in hexanes) dropwise. The reaction mixture was stirred at the same temperature for 30 min and then quenched by dropwise addition of potassium sodium tartrate (10% solution in water). The resulting mixture was warmed to room temperature and stirred vigorously for 1 h. The organic layer was separated and the aqueous phase extracted with diethyl ether. The combined organic layer was washed with brine, dried (MgSO₄) and concentrated in vacuo. The residue was purified by column chromatography on silica gel, eluting with 25% diethyl ether-hexane to give 46.1 (0.26 g, 65% yield) as a colorless viscous liquid.

¹H NMR (500 MHz, CDCl₃) δ 9.80 (s, 1H), 7.27 (t, J=7.5 Hz, 2H), 6.93 (d, J=7.5 Hz, 2H), 6.89 (d, J=7.5 Hz, 2H), 3.95 (t, J=6.2 Hz, 2H), 2.45 (t, J=7.2 Hz, 2H), 1.79 (quintet, J=6.7 Hz, 2H), 1.67 (quintet, J=7.0 Hz, 2H), 1.50 (quintet, J=6.7 Hz, 2H), 1.41 (quintet, J=7.7 Hz, 2H).

7-[4-(Benzyloxy)phenoxy]heptanal 46.2 was synthesized analogous to the preparation of 46.1 using 45.2/20.4 (0.624 g, 2 mmol) and diisobutylaluminum hydride (4.5 mL, 4.5 mmol, using a 1M solution in hexanes) in THF (20 mL). Purification by flash column chromatography on silica gel gave 46.2 (0.39 g, 63% yield) as a white solid (m p 65-67° C.).

¹H NMR (500 MHz, CDCl₃) δ 9.80 (s, 1H), 7.42 (d, J=7.2 Hz, 2H), 7.38 (t, J=7.2 Hz, 2H), 7.31 (t, J=7.2 Hz, 1H), 6.90 (d, J=8.7 Hz, 2H), 6.82 (d, J=8.7 Hz, 2H), 5.01 (s, 2H), 3.90 (t, J=6.2 Hz, 2H), 2.44 (dt, J=7.2 Hz, J=2.0 Hz, 2H), 1.76 (quintet, J=7.5 Hz, 2H), 1.67 (quintet, J=7.5 Hz, 2H), 1.48 (quintet, J=7.2 Hz, 2H), 1.40 (quintet, J=7.7 Hz, 2H).

### 7-Phenoxy-1-(5-methyl-1,3,4-oxadiazol-2-yl)-heptan-1-ol (48.1)

To a stirred solution of 47 (0.252 g, 3 mmol) in anhydrous THF (5 mL), at −78° C., under a nitrogen atmosphere, was added n-BuLi (1.2 mL, 3 mmol, using a 2.5 M solution in hexanes) dropwise. Stirring was continued for 15 min at −78° C. and then MgBr₂.Et₂O (0.774 g, 3 mmol) was added. The resulting mixture was warmed to −50° C. over a 2 hours period, and then it was transferred by cannula to a cooled (−78° C.) slurry of 46.1 (0.125 g, 0.6 mmol) and CeCl₃, (0.738 g, 3 mmol) in anhydrous THF (6 mL), which was previously stirred at room temperature for 2 hours under nitrogen. Following the addition, the resultant mixture was allowed to warm to room temperature over a 4 hours period. The reaction mixture was quenched with dropwise addition of 5% aqueous AcOH solution (10 mL), diluted with AcOEt (20 mL) and the organic phase was separated. The aqueous layer extracted with AcOEt, the combined organic layer was washed with aqueous saturated NaHCO₃ solution and brine, dried (MgSO₄) and the solvent was evaporated under reduced pressure. The residue obtained was purified by flash column chromatography on silica gel (75% ethyl acetate-hexane) to give 48.1 (92.5 mg, 53% yield) as a white solid (m p 50-52° C.).

¹H NMR (500 MHz, CDCl₃) δ 7.27 (t, J=7.7 Hz, 2H), 6.93 (t, J=7.7 Hz, 1H), 6.89 (d, J=7.7 Hz, 2H), 4.91 (t, J=6.2 Hz, 1H), 3.95 (t, J=6.7 Hz, 2H), 2.80 (br s, 1H), 2.54 (s, 3H), 1.98-1.90 (m, 2H), 1.78 (quintet, J=6.7 Hz, 2H), 1.54-1.40 (m, 6H).

7-(4-Benzyloxy-phenoxy)-1-(5-methyl-1,3,4-oxadiazol-2-yl)-heptan-1-ol (48.2). The synthesis was carried out analogous to the preparation of 48.1 using 46.2 (0.1 g, 0.32 mmol), cerium chloride (0.44 g, 1.6 mmol) and 47 (0.42 g, 1.6 mmol). Purification by flash column chromatography on silica gel gave pure 48.2 (0.077 mg, 55% yield) as a white solid (m p 98-100° C.).

1H NMR (500 MHz, CDCl₃) δ 7.42 (d, J=7.5 Hz, 2H), 7.38 (t, J=7.5 Hz, 2H), 7.31 (t, J=7.5 Hz, 1H), 6.90 (d, J=8.7 Hz, 2H), 6.82 (d, J=8.7 Hz, 2H), 5.01 (s, 2H), 4.90 (q, J=6.0 Hz, 1H), 3.89 (t, J=6.5 Hz, 2H), 2.54 (s, 3H), 2.51 (d, J=6.0 Hz, 1H), 2.00-1.92 (m, 2H), 1.75 (quintet, J=7.5 Hz, 2H), 1.56-1.40 (m, 6H).

### 7-Phenoxy-1-(5-methyl-1,3,4-oxadiazol-2-yl)heptan-1-one (49.1)

To a solution of 48.1 (64 mg, 0.22 mmol) in wet methylene chloride (5 mL) at room temperature, under nitrogen was added Dess-Martin periodinane (140 mg, 0.33 mmol) and the resulting suspension stirred for 2 hours. The reaction mixture was diluted with Na₂S₂O₃ (10% in H₂O) and saturated aqueous NaHCO₃ solution and the organic phase was separated. The aqueous layer was extracted with AcOEt and the combined organic layer was washed with brine, dried (MgSO₄) and evaporated under reduced pressure. The residue obtained was purified by flash column chromatography on silica gel (50% ethyl acetate-hexane) to give 49.1 (52 mg, 82% yield) as a white solid (m p 75-77° C.).

¹H NMR (500 MHz, CDCl₃) δ 7.27 (t, J=7.5 Hz, 2H), 6.93 (t, J=7.5 Hz, 1H), 6.89 (d, J=7.5 Hz, 2H), 3.95 (t, J=6.2 Hz, 2H), 3.15 (t, J=7.2 Hz, 2H), 2.64 (s, 3H), 1.84-1.77 (m, 4H), 1.52-1.44 (m, 4H).

7-(4-Benzyloxy-phenoxy)-1-(5-methyl-1,3,4-oxadiazol-2-yl)-heptan-1-one (49.2). The synthesis was carried out analogous to the preparation of 49.1 using 48.2 (60 mg, 0.15 mmol) and Dess-Martin periodinane (0.127 g, 0.3 mmol) in wet CH₂Cl₂ (5 mL). Purification by flash column chromatography on silica gel gave pure compound 49.2 (47.5 mg, 80% yield) as a white solid (m p 118-120° C.).

¹H NMR (500 MHz, CDCl₃) δ 7.42 (d, J=7.5 Hz, 2H), 7.38 (t, J=7.5 Hz, 2H), 7.31 (t, J=7.5 Hz, 1H), 6.90 (d, J=8.7 Hz, 2H), 6.82 (d, J=8.7 Hz, 2H), 5.01 (s, 2H), 3.90 (t, J=6.2 Hz, 2H), 3.14 (t, J=7.5 Hz, 2H), 2.64 (s, 3H), 1.84-1.74 (m, 4H), 1.54-1.44 (m, 4H).

7-(4-Hydroxy-phenoxy)-1-(5-methyl-1,3,4-oxadiazol-2-yl)-heptan-1-one (50). To a solution of 49.2 (30 mg, 0.076 mmol) in AcOEt (5 mL) was added 10% Pd/C (6 mg, 20% w/w) and the resulting suspension was stirred vigorously under hydrogen atmosphere, overnight at room temperature. The catalyst was removed by filtration through Celite, and the filtrate was evaporated under reduced pressure. The residue obtained was purified by flash column chromatography on silica gel (60% ethyl acetate-hexane) to give pure compound 50 (0.016 g, 71% yield) as a white solid (m p 134-135° C.).

¹H NMR (500 MHz, CDCl₃) δ 6.80-6.74 (m, 4H), 4.56 (br s, 1H), 3.89 (t, J=6.5 Hz, 2H), 3.14 (t, J=7.2 Hz, 2H), 2.64 (s, 3H), 1.84-1.74 (m, 4H), 1.54-1.44 (m, 4H).

α-Keto-oxadiazoles 54 and 57 (shown in Scheme 11) were synthesized by a method depicted in Scheme 11 starting from commercially available 3-benzyloxybromobenzene (28) and 3-anisaldehyde (55).

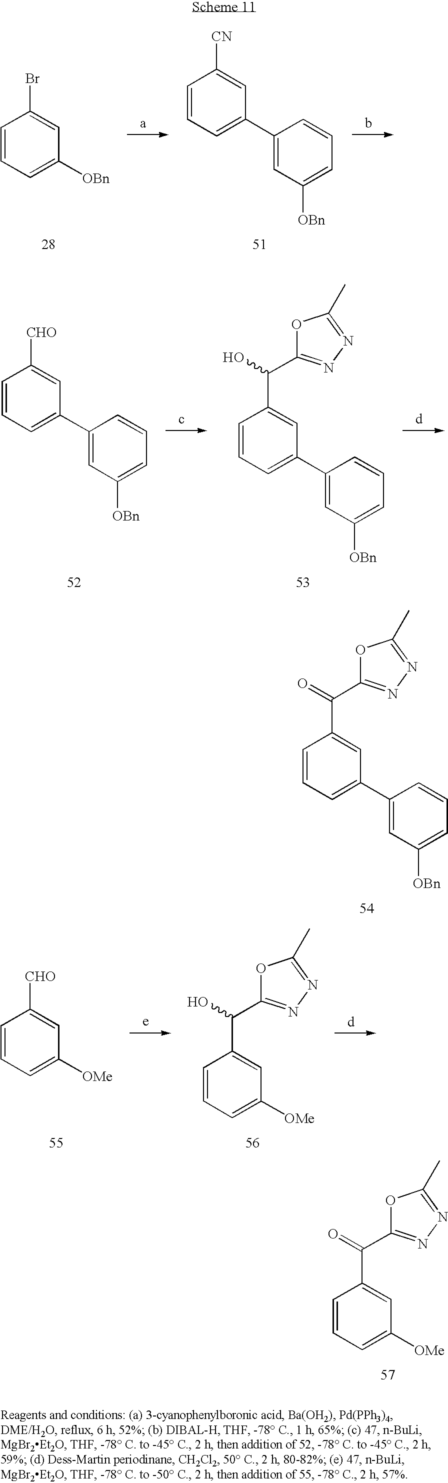

### Experimental Procedures

### 3-(3-Benzyloxy-phenyl)benzonitrile (51)

A degassed mixture of 3-benzyloxy-phenyl bromide (28) (0.2 g, 0.76 mmol), 3-cyanophenylboronic acid (0.223 g, 1.52 mmol), barium hydroxide (0.285 g, 1.67 mmol), Pd(PPh₃)₄ (0.088 g, 0.076 mmol), DME (5 mL) and H₂O (3 mL) was heated (80° C.) for 6 hours with vigorous stirring under an argon atmosphere. The reaction mixture was cooled to room temperature, diluted with ethyl acetate, and filtered through a plug of celite. The filtrate was diluted with brine; the organic phase was separated, dried (MgSO₄) and concentrated in vacuo. The residue obtained was purified by flash column chromatography (20% diethyl ether-hexane) to give 51 (0.130 g, 60% yield) as a viscous liquid.

¹H NMR (500 MHz, CDCl₃) δ 7.85 (t, J=2.5 Hz, 1H), 7.78 (dt, J=7.5 Hz, J=1.5 Hz, 1H), 7.63 (dt, J=8.0 Hz, J=1.5 Hz, 1H), 7.53 (t, J=8.0 Hz, 1H), 7.46 (d, J=7.5 Hz, 2H), 7.44-7.37 (m, 3H), 7.35 (t, J=7.0 Hz, 1H), 7.18-7.14 (m, 2H), 7.02 (dd, J=8.5 Hz, J=2.5 Hz, 1H), 5.17 (s, 2H).

### 3-(3-Benzyloxy-phenyl)benzaldehyde (52)

To a stirred solution of 51 (0.12 g, 0.42 mmol) in anhydrous THF (10 mL) at −78° C., under a nitrogen atmosphere was added diisobutylaluminum hydride (0.5 mL, 0.5 mmol, using a 1M solution in hexane) dropwise. The reaction mixture was stirred at the same temperature for 1 hour and then quenched by dropwise addition of potassium sodium tartrate (10% solution in water). The resulting mixture was warmed to room temperature, diluted with diethyl ether (20 mL) and stirred vigorously for 1 h. The organic phase was separated and the aqueous phase was extracted with diethyl ether. The combined organic layer was washed with brine, dried (MgSO₄) and evaporated under reduced pressure. The residue obtained was purified by flash column chromatography on silica gel (20% diethyl ether-hexane) to give 52 (0.091 g, 75% yield) as a viscous liquid.

¹H NMR (500 MHz, CDCl₃) δ 10.08 (s, 1H), 8.09 (t, J=1.5 Hz, 1H), 7.85 (dt, J=7.5 Hz, J=1.5 Hz, 2H), 7.60 (t, J=7.7 Hz, 1H), 7.47 (d, J=7.5 Hz, 2H), 7.44-7.32 (m, 3H), 7.35 (t, J=7.5 Hz, 1H), 7.27-7.21 (m, 2H), 7.02 (dd, J=7.7 Hz, J=2.0 Hz, 1H), 5.14 (s, 2H).

### 1-(3′-Benzyloxy-1,1′-biphenyl-3-yl)-1-(5-methyl-1,3,4-oxadiazol-2-yl)-methanol (53)

To a stirred solution of 47 (0.118 g, 1.5 mmol), in dry THF (5 mL), at −78° C., under a nitrogen atmosphere, was added n-BuLi (0.6 mL, 1.5 mmol, using a 2.5M solution in hexane) dropwise. Stirring continued for 10 min at −78° C. and then MgBr₂.Et₂O (0.4 g, 1.5 mmol) was added. The resulting mixture was warmed to −45° C. over a 2 hours period, and then it was cooled back to −78° C., and a solution of 52 (0.081 g, 0.28 mmol) in dry THF (5 mL) was added dropwise. Following the addition, the reaction mixture was warmed to −45° C. over a 2 hours period and then diluted with aqueous NH₄Cl solution (5 mL) and AcOEt (20 mL). The resulting mixture was gradually warmed to room temperature, the organic phase was separated and the aqueous phase extracted with AcOEt. The combined organic layer was washed with brine, dried (MgSO₄) and evaporated under reduced pressure. The residue obtained was purified by flash column chromatography on silica gel (75% ethyl acetate-hexane) to give 53 (0.052 g, 50% yield) as a colorless viscous liquid.

¹H NMR (500 MHz, CDCl₃) δ 7.69 (m as br s, 1H), 7.59-7.56 (m, 1H), 7.47-7.45 (m, 4H), 7.40 (t, J=7.0 Hz, 2H), 7.37-7.32 (m, 2H), 7.20 (t, J=2.0 Hz, 1H), 7.18 (d J=8.0 Hz, 1H), 6.98 (dd, J=8.5 Hz, J=2.0 Hz, 1H), 6.08 (s, 1H), 5.12 (s, 2H), 3.22 (br s, 1H), 2.45, (s, 3H).

### 1-(3′-Benzyloxy-1,1′-biphenyl-3-yl)-1-(5-methyl-1,3,4-oxadiazol-2-yl)-ketone (54)

To a solution of 53 (45 mg, 0.12 mmol) in wet CH₂Cl₂ (5 mL) at room temperature, under nitrogen, was added Dess-Martin periodinane (102 mg, 0.24 mmol) and the resulting suspension stirred for 2 hours at 50° C. The reaction mixture was cooled to room temperature, diluted with Na₂S₂O₃ (10% in H₂O) and saturated aqueous NaHCO₃ solution, and the organic phase was separated. The aqueous layer was extracted with AcOEt and the combined organic layer was washed with brine, dried MgSO₄) and evaporated under reduced pressure. The residue obtained was purified by flash column chromatography on silica gel (60% ethyl acetate-hexane) to give 54 (35.52 mg, 80% yield) as a white solid (m p 97-99° C.).

¹H NMR (500 MHz, CDCl₃) δ 8.70 (t, J=2.0 Hz, 1H), 8.54 (d, J=8.0 Hz, 1H), 7.90 (d, J=8.0 Hz, 1H), 7.62 (t, J=8.0 Hz, 1H), 7.48 (d, J=7.5 Hz, 2H), 7.42-7.40 (m, 3H), 7.34 (t, J=7.5 Hz, 1H), 7.27-7.25 (m, 2H), 7.01 (dd, J=7.0 Hz, J=2.0 Hz, 1H), 5.15 (s, 2H), 2.71 (s, 3H).

1-(3-Methoxy-phenyl)-1-(5-methyl-1,3,4-oxadiazol-2-yl)-methanol (56). The synthesis was carried out analogous to the preparation of 53 using 56 (0.14 g, 1.03 mmol) and 47 (0.29 g, 3.45 mmol). Purification by flash column chromatography on silica gel gave compound 55 (0.12 g, 53.4% yield) as a viscous liquid.

1H NMR (500 MHz, CDCl₃) δ 7.30 (t, J=7.5 Hz, 1H), 7.06-7.02 (m, 2H), 6.90 (dd, J=7.5 Hz, J=2.5 Hz, 1H), 6.05 (d, J=5.0 Hz, 1H), 3.83 (s, 3H), 3.67 (d, J=5.0 Hz, 1H), 2.49 (s, 3H).

1-(3-Methoxy-phenyl)-1-(5-methyl-1,3,4-oxadiazol-2-yl)-ketone (57) was synthesized as in 54 using 56 (0.1 g, 0.454 mmol) and Dess-Martin periodinane (0.38 g, 0.9 mmol) in wet CH₂Cl₂ (10 mL). Purification by flash column chromatography on silica gel gave compound 57 (0.080 g, 82% yield) as a viscous liquid.

¹H NMR (500 MHz, CDCl₃) δ 8.10 (dt, J=7.5 Hz, J=1.5 Hz, 1H), 7.99 (t, J=1.5 Hz, 1H), 7.47 (t, J=7.5 Hz, 1H), 7.24 (dd, J=7.5 Hz, J=1.5 Hz, 1H), 3.90 (s, 3H), 2.70 (s, 3H).

### 7. Synthesis of Saccharin Analogs

Saccharin analogs 59.1, 59.2, 59.3 and 60 (shown in Scheme 12) were synthesized by a method depicted in Scheme 12 starting from commercially available saccharin (58) and the appropriate bromide.

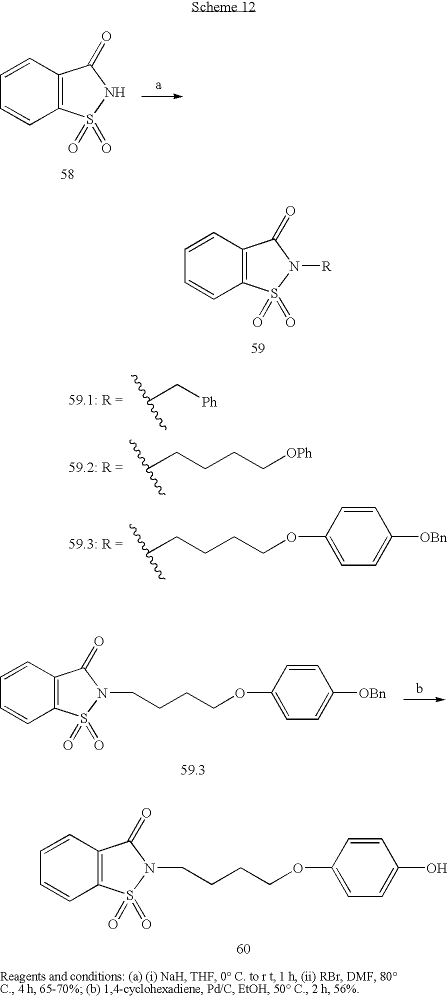

### Experimental Procedures

### N-(Phenylmethyl)saccharin (59.1)

To a stirred solution of saccharin 58 (0.154 g, 0.75 mmol) in anhydrous THF (10 mL) at 0° C., under nitrogen atmosphere was added NaH (0.019 g, 0.8 mmol, using a 60% dispersion in mineral oil) and the resulting slurry was gradually warmed to room temperature over 1 hour period. Solvent was removed under reduced pressure, and the saccharin sodium salt was dissolved in DMF (5 mL). To this solution, was added a solution of benzyl bromide (0.051 g, 0.3 mmol) in DMF (5 mL), under nitrogen, at room temperature and the mixture warmed to 80° C. and stirred for 4 hours. The reaction mixture was cooled to room temperature and diluted with dropwise addition of water (5 mL) and AcOEt (20 mL). The organic layer was separated and the aqueous layer extracted with AcOEt. The combined organic layer was washed with brine, dried (MgSO₄) and the solvent removed in vacuo. The residue obtained was purified by flash column chromatography on silica gel (50% diethyl ether-hexane) to give 59.1 (0.054 g, 66% yield), as a white solid (m p 106-108° C.).

¹H NMR (500 MHz, CDCl₃) δ 8.06 (d, J=7.0 Hz, 1H), 7.93 (d, J=7.0 Hz, 1H), 7.87 (td, J=7.0 Hz, J=1.2 Hz, 1H), 7.83 (td, J=7.0 Hz, J=1.2 Hz, 1H), 7.51 (d, J=7.5 Hz, 2H), 7.36 (t, J=7.5 Hz, 2H), 7.31 (t, J=7.0 Hz, 1H), 4.91 (s, 2H).

### N-(4-Phenoxy-butyl)saccharin (59.2)

The synthesis was carried out analogous to the preparation of 59.1 using 58 (0.23 g, 1.25 mmol), NaH (0.030 g, 1.25 mmol) and 4-phenoxy-butyl bromide (0.115 g, 0.5 mmol) in DMF (5 mL). Purification by flash column chromatography on silica gel gave 59.2 (0.1 g, 67% yield) as a white solid (m p 92-94° C.).

¹H NMR (500 MHz, CDCl₃) δ 8.07 (d, J=7.0 Hz, 1H), 7.93 (d, J=7.0 Hz, 1H), 7.87 (td, J=7.0 Hz, J=1.2 Hz, 1H), 7.83 (td, J=7.0 Hz, J=1.2 Hz, 1H), 7.27 (t, J=7.5 Hz, 2H), 6.93 (t, J=7.5 Hz, 1H), 6.90 (d, J=7.5 Hz, 2H), 4.02 (t, J=6.5 Hz, 2H), 3.88 (t, J=7.2 Hz, 2H), 2.07 (quintet, J=6.9 Hz, 2H), 1.92 (quintet, J=6.9 Hz, 2H).

### N-[4-(4-Benzyloxy-phenoxy)-butyl]saccharin (59.3)

The synthesis was carried out analogous to the preparation of 59.1 using 58 (0.307 g, 1.5 mmol), NaH (0.036 g, 1.5 mmol) and 4-(4-benzyloxy-phenoxy)-butyl bromide (0.2 g, 0.6 mmol) in DMF (5 mL). Purification by flash column chromatography on silica gel gave 59.3 (0.150 g, 66% yield) as a white solid (m p 82-84° C.).

¹H NMR (500 MHz, CDCl₃) δ 8.06 (d, J=7.0 Hz, 1H), 7.92 (d, J=7.0 Hz, 1H), 7.87 (td, J=7.0 Hz, J=1.2 Hz, 1H), 7.83 (td, J=7.0 Hz, J=1.2 Hz, 1H), 7.42 (d, J=7.5 Hz, 2H), 7.37 (t, J=7.5 Hz, 2H), 7.31 (t, J=7.5 Hz, 1H), 6.89 (d, J=9.0 Hz, 2H), 6.82 (d, J=9.0 Hz, 2H), 5.06 (s, 2H), 3.97 (t, J=6.4 Hz, 2H), 3.86 (t, J=7.2 Hz, 2H), 2.05 (quintet, J=6.9 Hz, 2H), 1.89 (quintet, J=6.9 Hz, 2H).

### N-[4-(4-Hydroxy-phenoxy)-butyl]saccharin (60)

To a stirred solution of 59.3 (0.1 g, 0.23 mmol) in EtOH (5 mL) was added 10% Pd/C (0.1 g, 100% w/w) and 1,4-cyclohexadiene (92 mg, 1.15 mmol) and the resulting suspension was stirred vigorously at 50° C. for 2 hours. The reaction mixture was cooled to room temperature, the catalyst was removed by filtration through Celite, and the filtrate was evaporated under reduced pressure. The residue obtained was purified by flash column chromatography on silica gel (60% diethyl ether-hexane) to give 60 (0.044 g, 56% yield) as a white solid (m p 107-109° C.).

¹H NMR (500 MHz, CDCl₃) δ 8.06 (d, J=7.0 Hz, 1H), 7.92 (d, J=7.0 Hz, 1H), 7.87 (td, J=7.0 Hz, J=1.2 Hz, 1H), 7.83 (td, J=7.0 Hz, J=1.2 Hz, 1H), 6.79 (d, J=9.0 Hz, 2H), 6.74 (d, J=9.0 Hz, 2H), 4.38 (br s, 1H), 3.96 (t, J=6.4 Hz, 2H), 3.89 (t, J=7.2 Hz, 2H), 2.05 (quintet, J=6.9 Hz, 2H), 1.89 (quintet, J=6.9 Hz, 2H).

### 8. Synthesis of α-keto-esters and α,α-difluoromethyl-ketones

α-Keto-esters 63.1 and 63.2 (shown in Scheme 13) as well as 3,3-difluoro-8-phenoxy-2-octanone (65, shown in Scheme 13) were synthesized by the methods depicted in Scheme 13 starting from commercially available 3-benzyloxy-phenol (61) and 5-phenoxypentyl bromide (62.1).

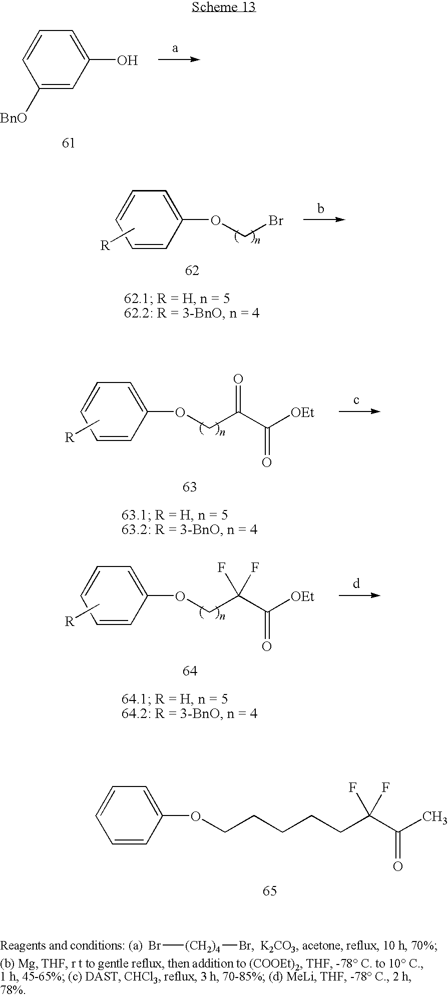

### Experimental Procedures

### 1-Bromo-4-[4-(benzyloxy)phenoxy]butane (62.2)

A mixture of 3-benzyloxyphenol (1 equiv.), dibromobutane (1.5 equiv) and anhydrous potassium carbonate was stirred and heated to refluxed in dry acetone for 10 hours, then it was cooled to room temperature and solid materials were filtered off. The filtrate was evaporated, water was added to the residue and the mixture was extracted with diethyl ether. The ethereal layer was washed with 10% sodium hydroxide solution, water and brine, dried (MgSO₄) and concentrated under reduced pressure. Purification by flash column chromatography on silica gel (10% diethyl ether-hexane) gave title compound as colorless viscous oil in 70% yield.

### α-Keto-esters (63)

To a three-neck round bottom flask containing Mg turnings (1.2 equiv.) equipped with a magnetic stirrer and dimroth condenser was added a solution of alkyl bromide 62 (1 equiv.) in anhydrous THF via syringe and external heating under argon atmosphere. The reaction mixture was refluxed gently for 30-40 min. and then it was cooled to room temperature, before conveying it to a dropping funnel. The Grignard reagent was added drop wise to a solution of diethyl oxalate (1.5 equiv) in THF at −78° C. The reaction mixture was warmed to 10° C. within 1 hour and then was quenched by the addition of saturated ammonium chloride solution. The organic layer was separated, the aqueous layer was extracted with diethyl ether and the combined organic layer was washed with brine, dried over MgSO₄ and evaporated. The residue obtained was purified by flash column chromatography on silica gel (diethyl ether-hexane) to give pure compound 63 in 45-65% yield.

### Selected data of synthesized α-Keto-esters (63)

2-Oxo-7-phenoxy-heptanoic acid ethyl ester (63.1). ¹H NMR (500 MHz, CDCl₃) δ 7.27 (t, J=7.6 Hz, 2H), 6.93 (t, J=7.6 Hz, 1H), 6.88 (d, J=7.6 Hz, 2H), 4.32 (q, J=7.5 Hz, 2H), 3.96 (t, J=6.0 Hz, 2H), 2.88 (t, J=7.5 Hz, 2H), 1.81 (qt, J=6.5 Hz, 2H), 1.72 (qt, J=7.5 Hz, 2H), 1.56-1.49 (m, 2H), 1.37 (t, J=7.5 Hz, 3H).

### α,α-Difluoro-esters (64)

To a stirred solution of α-keto-ester 63 (1 equiv.) in anhydrous chloroform at room temperature under an argon atmosphere was added diethylaminosulfur trifluoride (1.1 equiv.). The reaction mixture was heated under gentle reflux for 3 hours then it was cooled to room temperature and poured into ice-water. The organic layer was separated, washed with sat. NaHCO₃ solution and dried over MgSO₄. Volatiles were removed under reduced pressure and the crude product was purified by flash chromatography on silica gel (diethyl ether-hexane) to give pure compound 64 in 70-85% yield.

### 3,3-Difluoro-8-phenoxy-2-octanone (65)

To a solution of α,α-difluoro-ester 64.1(1 equiv.) in anhydrous THF at −78° C. under an argon atmosphere was added methyl lithium (1.5 equiv., using a 1.6 M solution in diethyl ether) dropwise. The reaction mixture was stirred at −78° C. for 2 hours and then it was quenched by the addition of saturated ammonium chloride solution. The organic phase was separated, the aqueous layer was extracted with diethyl ether and the combined organic layer was washed with water and brine, dried (MgSO₄) and evaporated under reduced pressure. Purification by flash column chromatography on silica gel (5% diethyl ether-hexane) gave the title compound as colorless oil in 78% yield.

3,3-Difluoro-8-phenoxy-2-octanone (65). ¹H NMR (500 MHz, CDCl₃) δ 7.28 (m as t, J=7.5 Hz, 2H), 6.93 (m as t, J=7.5 Hz, 1H), 6.89 (m as d, J=7.5 Hz, 2H), 3.96 (t, J=6.3 Hz, 2H), 2.33 (t, J=1.5 Hz, 3H), 2.06-1.95 (m, 2H), 1.83-1.77 (m, 2H), 1.56-1.51 (m, 4H).

While preferred embodiments of the foregoing invention have been set forth for purposes of illustration, the foregoing description should not be deemed a limitation of the invention herein. Accordingly, various modifications, adaptations and alternatives may occur to one skilled in the art without departing from the spirit and scope of the present invention.

What is claimed:

1. A method for treating excitotoxicity in a subject comprising:
providing at least one endocannabinoid/anandamide transport inhibitor or a physiologically acceptable salt thereof;
providing at least one fatty-acid amide hydrolase inhibitor or a physiologically acceptable salt thereof;
combining said endocannabinoid/anandamide transport inhibitor and said fatty-acid amide hydrolase inhibitor into a therapeutically effective medicament; and
administering said medicament to the subject.

2. The method of claim 1 wherein said at least one endocannabinoid/anandamide transport inhibitor has the following structural formula
X′—Y′—Z′ (I)
wherein:
X′ is selected from a fatty acid hydrophobic carbon chain containing from about 4 to about 30 carbon atoms having one or more nonconjugated cis double bonds in the middle portion of the chain and a terminal moiety selected from hydrogen, aryl, alkyl aryl, or aryl substituted with a member selected from the group consisting of hydroxy, halogen, —NO₂, —NH₂, —CH₃, —OCH₃ and —SCH₃; biphenyl; biphenyl substituted with between 1-6 substituents including OH, CH₃, halogen, SCH₃, NH₂, NHCOR, SO₂NHR, and NO₂; and biphenyl having an straight alkyl or branched alkyl group of about 1 to about 10 carbon atoms;
Y′ is selected from hydrogen, —O—, —S—, —NH—, —NH—C(O)—, —NH—C(O)—NH—, —NH—C(O)O—, —C(O)—NH—, —O—C(O)—, —O—C(O)—NH—, —C(O)—C(O)—NH, —NH—C(O)—C(O)—, —O—C(O)—O—, and —C(O)—O—;
Z′ is selected from the group consisting of hydrogen, aryl, substituted aryl, alkyl, hydroxy alkyl, alkyl aryl, hydroxy aryl, halogen substituted alkyl aryl, heterocyclics, hydroxy heterocyclic, cyclic glycerols and substituted cyclic glycerols, COCF₃, C(O)-alcohol, —(CH₂)ₘ—(C(CH₃)₂)ₚ—(CH₂)ₙ-T₂-T₃, —(CH₂)ₘ—(CH(CH₃))_{q}—(CH₂)ₙ-T₂-T₃;
where m and n are each independently selected from an integer between 0 through 6;
p and q are each independently 0 or 1;
T₂ is optionally present and is selected from aryl, a cyclic ring, a bicyclic ring, a tricyclic ring, a heterocyclic ring, a heterobicyclic ring, a heterotricyclic ring, a heteroaromatic ring, 1- or 2-glycerol, 1- or 2-cyclic glycerol, alkyl, alkenyl, and alkynyl; and
T₃ is selected from H, OH, SH, halogen, C(halogen)₃, CH(halogen)₂, O-alkyl, N₃, CN, NCS, NH₂, alkylamino, and dialkylamino.

3. The method of claim 2 wherein X′ has the structural formula II
CR′₃—(CR₂)ₐ-(cis-CH═CHCR₂)_{b}—(CR₂)_{c}— (II)
wherein:
R is selected from hydrogen and a straight or branched alkyl group having between 1 to 5 carbons;
R′ is selected from H; alkyl; unsubstituted phenyl; phenyl substituted with a member selected from the group consisting of hydroxyl, halogen, —NO₂, —NH₂, —SCH₃, —CH₃ and —OCH₃; unsubstituted biphenyl; biphenyl substituted with a member from the group consisting of hydroxyl, halogen, —NO₂, —NH₂, —SCH₃, —CH₃ and —OCH₃;
a and c are each independently selected from 0 and an integer between 1 through 10; and
b is an integer from 1 to 6.

4. The method of claim 1 wherein said at least one endocannabinoid/anandamide transport inhibitor is substantially pure.

5. A method for treating excitotoxicity in a subject comprising:
providing at least one endocannabinoid/anandamide transport inhibitor or a physiologically acceptable salt thereof, wherein said at least one endocannabinoid/anandamide transport inhibitor has the following structural formula
X′—Y′—Z′ (I)
wherein:
X′ is selected from a fatty acid hydrophobic carbon chain containing from about 4 to about 30 carbon atoms having one or more nonconjugated cis double bonds in the middle portion of the chain and a terminal moiety selected from hydrogen, aryl, alkyl aryl, or aryl substituted with a member selected from the group consisting of hydroxy, halogen, —NO₂, —NH₂, —CH₃, —OCH₃ and —SCH₃;
biphenyl; biphenyl substituted with between 1-6 substituents including OH, CH₃, halogen, SCH₃, NH₂, NHCOR, SO₂NHR, and NO₂; and biphenyl having an straight alkyl or branched alkyl group of about 1 to about 10 carbon atoms;
Y′ is selected from hydrogen, —O—, —S—, —NH—, —NH—C(O)—, —NH—C(O)—NH—, —NH—C(O)O—, —C(O)—NH—, —O—C(O)—, —O—C(O)—NH—, —C(O)—C(O)—NH, —NH—C(O)—C(O)—, —O—C(O)—O—, and —C(O)—O—;
Z′ is selected from the group consisting of hydrogen, aryl, substituted aryl, alkyl, hydroxy alkyl, alkyl aryl, hydroxy aryl, halogen substituted alkyl aryl, heterocyclics, hydroxy heterocyclic, cyclic glycerols and substituted cyclic glycerols, COCF₃, C(O)-alcohol, —(CH₂)ₘ—(C(CH₃)₂)ₚ—(CH₂)ₙ-T₂-T₃, —(CH₂)ₘ—(CH(CH₃))_{q}—(CH₂)ₙ-T₂-T₃;
where m and n are each independently selected from an integer between 0 through 6;
p and q are each independently 0 or 1;
T₂ is optionally present and is selected from aryl, a cyclic ring, a bicyclic ring, a tricyclic ring, a heterocyclic ring, a heterobicyclic ring, a heterotricyclic ring, a heteroaromatic ring, 1- or 2-glycerol, 1- or 2-cyclic glycerol, alkyl, alkenyl, and alkynyl; and
T₃ is selected from H, OH, SH, halogen, C(halogen)₃, CH(halogen)₂, O-alkyl, N₃, ON, NCS, NH₂, alkylamino, and dialkylamino;
providing at least one fatty-acid amide hydrolase inhibitor or a physiologically acceptable salt thereof;
wherein said at least one fatty-acid amide hydrolase inhibitor has the following structural formula
R—X—Y (III)
wherein:
R is selected from an alkyl group, a substituted alkyl group, an aryl group, a substituted aryl group, a heteroaryl group, a substituted heteroaryl group, a heterocyclic group and a substituted heterocyclic group;
X is selected from a straight chain hydrocarbyl group or a substituted straight chain hydrocarbyl group containing from about 4 to about 24 carbon atoms, a substituted aryl group, a heteroaryl group, a substituted heteroaryl group, a heterocyclic group or a substituted heterocyclic group; and
Y is a moiety capable of irreversibly binding with a nucleophilic group at the active site of an amidase enzyme;
combining said endocannabinoid/anandamide transport inhibitor and said fatty-acid amide hydrolase inhibitor into a therapeutically effective medicament; and
administering said medicament to the subject.

6. The method of claim 5 wherein Y is selected from 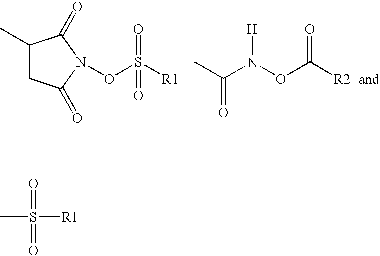
where:
R1 is selected from the group consisting of —F and —O-alkyl, where alkyl is a straight or branched moiety having between 1 to 4 carbons; and
R2 is a C1 to C4 straight or branched chain alkyl group.

7. The method of claim 5 wherein the FAAH inhibitor is: 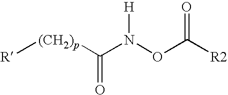
wherein:
R′ is selected from an aryl group, a substituted aryl group, a heteroaryl group, a substituted heteroaryl group, a heterocyclic group and a substituted heterocyclic group;
R2 is a C1 to C4 straight or branched chain alkyl group; and
p is an integer from about 6 to about 18.

8. The method of claim 5 wherein said at least one inhibitor is substantially pure.

9. The method of claim 5 wherein said excitotoxicity is associated with a condition selected from at least one of stroke, brain injury, brain trauma, hypoxia, ischemia, toxin exposure, tumor growth and excitotoxicity linked to dementia such as in Alzheimer's Disease.

10. The method of claim 1 wherein said at least one endocannabinoid/anandamide transport inhibitor has the following structural formula
X′—Y′—Z′ (I)
wherein:
X′ is selected from a fatty acid hydrophobic carbon chain containing from about 4 to about 30 carbon atoms having one or more nonconjugated cis double bonds in the middle portion of the chain and a terminal moiety selected from hydrogen, aryl, alkyl aryl, or aryl substituted with a member selected from the group consisting of hydroxy, halogen, —NO₂, —NH₂, —CH₃, —OCH₃ and —SCH₃; biphenyl; biphenyl substituted with between 1-6 substituents including OH, CH₃, halogen, SCH₃, NH₂, NHCOR, SO₂NHR, and NO₂; and biphenyl having an straight alkyl or branched alkyl group of about 1 to about 10 carbon atoms;
Y′ is selected from hydrogen, —O—, —S—, —NH—, —NH—C(O)—, —NH—C(O)—NH—, —NH—C(O)O—, —C(O)—NH—, —O—C(O)—, —O—C(O)—NH—, —C(O)—C(O)—NH, —NH—C(O)—C(O)—, —O—C(O)—O—, and —C(O)—O—;
Z′ is selected from the group consisting of hydrogen, aryl, substituted aryl, alkyl, hydroxy alkyl, alkyl aryl, hydroxy aryl, halogen substituted alkyl aryl, heterocyclics, hydroxy heterocyclic, cyclic glycerols and substituted cyclic glycerols, COCF₃, C(O)-alcohol, —(CH₂)ₘ—(C(CH₃)₂)ₚ—(CH₂)ₙ-T₂-T₃, —(CH₂)ₘ—(CH(CH₃))_{q}—(CH₂)ₙ-T₂-T₃;
where m and n are each independently selected from an integer between 0 through 6;
p and q are each independently 0 or 1;
T₂ is optionally present and is selected from aryl, a cyclic ring, a bicyclic ring, a tricyclic ring, a heterocyclic ring, a heterobicyclic ring, a heterotricyclic ring, a heteroaromatic ring, 1- or 2-glycerol, 1- or 2-cyclic glycerol, alkyl, alkenyl, and alkynyl; and
T₃ is selected from H, OH, SH, halogen, C(halogen)₃, CH(halogen)₂, O-alkyl, N₃, ON, NCS, NH₂, alkylamino, and dialkylamino;
and wherein said at least one fatty-acid amide hydrolase inhibitor has the following structural formula
R—X—Y (VI)
wherein:
Y is selected from the following structures, 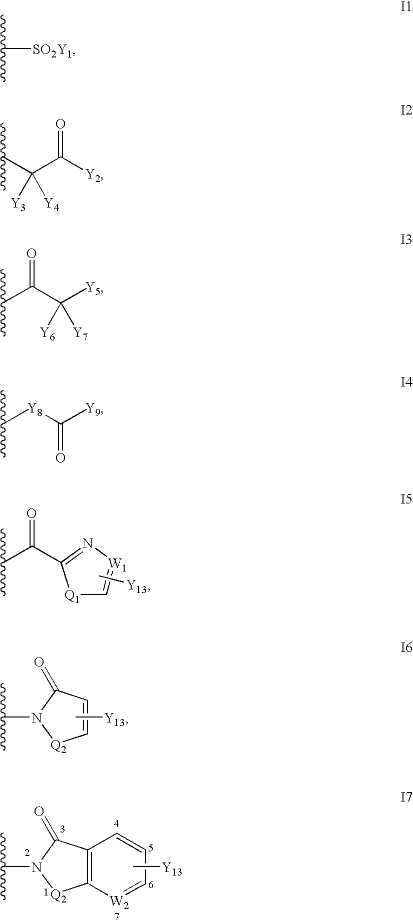
Y₁ is selected from —F, —Cl, —O-alkyl, —O-cycloalkyl, —O-heterocyclic, —O-aryl, —O-heteroaryl and —O-adamantlyl;
Y₂ is selected from —H, —OH, —NH₂, —OMe, —OEt, —CF₃, —C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄, -aryl-Y₁₄, -heteroaryl-Y₁₄, -cycloalkyl-Y₁₄, -heterocyclic-Y₁₄ and -adamantyl-Y₁₄;
Y₃ and Y₄ are each independently selected from —F, —Cl and —OH or Y3 and Y4 together with the common carbon atom form the structure >0=0;
Y₅ is selected from —F, —CONH₂, —SO₂NH₂, —COOH, —COOMe, —COOEt, —CF₃,
—C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄, -aryl-Y₁₄, -heteroaryl-Y₁₄, -cycloalkyl-Y₁₄, -adamantyl-Y₁₄ and -heterocyclic-Y₁₄;
Y₆ and Y₇ are each independently selected from —F, —CL and —OH;
Y₈ is selected from >NH and —O—;
Y₉ is selected from —OY₁₀ and —N(Y₁₁)Y₁₂;
Y₁₀ is selected from alkyl, aryl, heteroaryl, cycloalkyl, adamantyl, heterocyclic, —C₁₋₅-alkyl-Y₁₄, -aryl-Y₁₄, -heteroaryl-Y₁₄, -cycloalkyl-Y₁₄, -adamantyl-Y₁₄ and -heterocyclic-Y₁₄;
Y₁₁ is —H;
Y₁₂ is selected from alkyl, aryl, heteroaryl, cycloalkyl, adamantyl, heterocyclic, —O₁₋₆-alkyl-Y₁₄, —O₁₋₆-alkyl-aryl, —O₁₋₆-alkyl-heteroaryl, -aryl-Y₁₄, -heteroaryl-Y₁₄, -cycloalkyl-Y₁₄, -adamantyl-Y₁₄ and -heterocyclic-Y₁₄, or Y₁₁ and Y₁₂ together comprise part of a 5 or 6 membered saturated heterocyclic ring containing up to one additional heteroatom selected from N, O and S;
Y₁₃ is selected from —H, —OH, —SH, —NH₂, —CN, —N₃, —NCS, —NCO, —CONH₂, —SO₂NH₂, —COOH, —COOMe, —COOEt, —NO₂, —CF₃, —SO₃H, —P(O)(OH)₂, —CH₂—C≡CH, —CH═CH₂, fluoroalkyl, —O₁₋₆-alkyl, aryl, heteroaryl, cycloalkyl, adamantyl, heterocyclic, —O₁₋₆-alkyl-Y₁₄, -aryl-Y₁₄, -heteroaryl-Y₁₄, -cycloalkyl-Y₁₄, -adamantyl-Y₁₄ and -heterocyclic-Y₁₀;
Y₁₄ is selected from —OH, —SH, —NH₂, —CN, —N₃, —NCS, —NCO, —CONH₂, —SO₂NH₂, —COOH, —COOMe, —COOEt, —NO₂, —CF₃, —SO₃H, —P(O)(OH)₂, —CH₂—C≡CH and —CH═CH₂;
W₁ is selected from CH and N if Y₁₃ is not bonded to W₁, or W₁ is C if Y₁₃ is bonded to W₁;
W₂ is selected from CH and N if W₂ is not bonded to Y₁₃, or W₂ is C if W₂ is bonded to Y₁₃, if W₂ is N then it can occupy any position selected from 4, 5, 6 and 7 in structure 17;
Q₁ is selected from >CH₂, >O, >S and >NH if Q₁ is not bonded to Y₁₃, or Q₁ is selected from >CH and >N if Q₁ is bonded to Y₁₃;
Q₂ is selected from >SO₂, >C(O) and >S(O);
X is selected from —(CH₂)ₙ— and —(CH₂)ⱼ-A-(CH₂)ₖ—;
A is selected from —CH═CH—, —C≡C—, >C═O, >O, >S and >NH;
n is an integer from 0 to about 15;
j is an integer from 0 to about 10;
k is an integer from 0 to about 10;
R is selected from the following structures: 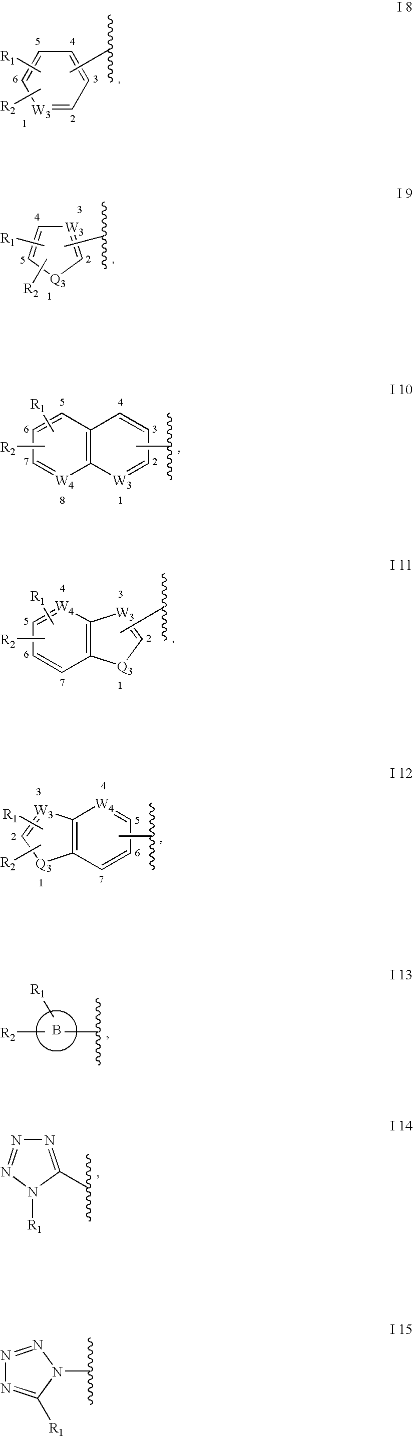
W₃ is selected from CH and N if W₃ is not bonded to X or R₁ or R₂, or W₃ is C if W₃ is bonded to X or R₁ or R₂, if W₃ is N then it can occupy any position selected from 1, 2, 3, 4, 5 and 6 in structure I 8; 2, 3, 4 and 5 in structure I 9; 1, 2, 3 and 4 in structure I 10; 2 and 3 in structure I 11 or 2 and 3 in structure I 12;
W₄ is selected from CH, N if W₄ is not bonded to X or R₁ or R₂, or W₄ is C if W₄ is bonded to X or R₁ or R₂, if W₄ is N then it can occupy any position selected from 5, 6, 7 and 8 in structure I 10; 4, 5, 6 and 7 in structure I 11 or 4, 5, 6 and 7 in structure I 12;
Q₃ is selected from >CH₂, >O, >S and >NH if Q₃ is not bonded to X or R₁ or R₂, or Q₃ is selected from >CH and >N if Q₃ is bonded to X or R₁ or R₂;
B is an adamantyl ring or a heteroadamantyl ring;
R₁ and R₂ are each independently selected from —H, —F, —Cl, —Br, —I, —OH, —SH, —NH₂, —CN, —N₃, —NCS, —NCO, —CONH₂, —SO₂NH₂, —COOH, —NO₂, —CHO, —CF₃, —SO₃H, —SO₂Cl, —SO₂F, —O—P(O)(OH)₂, —O—P(O)(O-alkyl)₂, —O—P(O)(OH)(O-alkyl), —P(O)(O-alkyl)₂,
—P(O)(OH)(O-alkyl), —Sn(alkyl)₃, —Si(alkyl)₃, —C≡CH, —CH₂—C≡CH, —CH═CH₂, -alkyl-R₃,
-cycloalkyl-R₃, -heterocyclic-R₃, -aryl-R₃, -heteroaryl-R₃, -alkyl-cycloalkyl-R₃, -alkyl-heterocyclic-R₃, -alkyl-aryl-R₃, -alkyl-heteroaryl-R₃, —Z-alkyl-R₃, —Z-cycloalkyl-R₃, —Z-heterocyclic-R₃, —Z-aryl-R₃, —Z-heteroaryl-R₃, —Z-alkyl-cycloalkyl-R₃, —Z-alkyl-heterocyclic-R₃, —Z-alkyl-aryl-R₃, —Z-alkyl-heteroaryl-R₃, -aryl-Z-alkyl-R₃, -aryl-Z-cycloalkyl-R₃, -aryl-Z-heterocyclic-R₃, -aryl-Z-aryl-R₃, -aryl-Z-heteroaryl-R₃, -aryl-Z-alkyl-cycloalkyl-R₃, -aryl-Z-alkyl-heterocyclic-R₃, -aryl-Z-alkyl-aryl-R₃, -aryl-Z-alkyl-heteroaryl-R₃, —CH(alkyl-R₃)₂,
—C(alkyl-R₃)₃, —N(alkyl-R₃)₂, —C(O)N(alkyl-R₃)₂ and —SO₂N(alkyl-R₃)₂;
Z is selected from —O, —S, —NH, —C(O), —C(O)O, —OC(O), —C(O)NH, —NHC(O), —SO, —SO₂, —SO₂NH, —NHSO₂, —SO₂O and —OSO₂;
R₃ is selected from —H, —F, —Cl, —Br, —I, -Me, -Et, —OH, —OAc, —SH, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —CH₂—C≡CH and —CH═CH₂.

11. The method of claim 10 wherein if Y is —SO₂—Y₁ (structure I 1) where Y₁ is F or O-alkyl and X is —(CH₂)ₙ— where n=4-15 or —(CH₂)ⱼ-A-(CH₂)ₖ—, where A is selected from O, S, —CH═CH— and j and k are each a positive integer such that the sum of j and k is equal to 4-15 and R is selected from structures I 8, I 9, I 10, I 11 or I 12 where R₁ is H; then R₂ can not be H, F, Cl, Br, I, NO₂, CF₃, CN, CHO, -aryl-R₃, -heteroaryl-R₃, —O-alkyl-R₃, —O-aryl-R₃, —C(O)—O-alkyl-R₃, —C(O)-alkyl-R₃, —C(O)NH-alkyl-R₃, —C(O)N(alkyl-R₃)₂ or —S-alkyl-R₃, where R₃═H; or
if Y is structure I 3 where Y₅ is F, Y₆ is F, Y₇ is F and X is —(CH₂)ₙ— where n=5-7; then R can not be phenyl, 2-hexyl-phenyl, 3-hexyl-phenyl, 4-heptyl-phenyl or 2-octyl-phenyl; or
if Y is structure I 3 where Y₅ is F, Y₆ is F, Y₇ is F and X is —(CH₂)ₙ— where n=3; then R can not be 2-butyl-naphthyl; or
if Y is structure I 4 where Y₈ is NH and Y₉ is OY₁₀ where Y₁₀ is alkyl, phenyl, pyridyl or C₁₋₅-alkyl-Y₁₄ where Y₁₄═NH₂ or NO₂ and X is —(CH₂)ₙ— where n=0-3; then R can not be naphthyl, indolyl or structure I 8 where W₁ is CH and R₁ and R₂ are each selected from O—C₁₋₁₆-alkyl, O—C₁₋₁₆-alkyl-phenyl, O—O₁₋₁₆-alkyl-pyridyl, phenyl, O-phenyl, O-pyridyl or C(O)NH—O₁₋₁₆-alkyl; or
if Y is structure I 5 where W₁ is CH or N, Q₁ is O or S, Y₁₃ is H, C₁₋₆-alkyl, aryl or heteroaryl and X is —(CH₂)ₙ— where n=3-9 or X is —(CH₂)ⱼ-A-(CH₂)ₖ— where A is O, S or NH and the sum of j and k is equal to 2-8; then R cannot be aryl; or
if Y is structure I 5 where W₁ is N, Q₁ is O or S, Y₁₃ is selected from phenyl, 2-pyridyl, 3-pyridyl, 4-pyridyl and 2-furyl and X is —(CH₂)ₙ— where n=5-8; then R can not be structure I 8 where W₁ is CH, R₁ is H and R₂ is H; or
if Y is structure I 5 where W₁ is CH, Q₁ is O or S, Y₁₃ is selected from phenyl, 2-pyridyl, 3-pyridazinyl, 4-pyrimidinyl, 2-pyrimidinyl, 5-pyrimidinyl, 3-pyrazinyl, 2-thiophenyl, 2-furyl, 2-thiazolyl or 2-oxazolyl and X is —(CH₂)ₙ— where n=1-10 then R can not be I 8 where W₁ is CH, R₁ is H and R₂ is H; or
if Y is structure I 4 where Y₈ is O and Y₉ is N(Y₁₁)Y₁₂ and X is (CH₂)ₙ— where n=0-3 then R can not be selected from structures I 8, I 9, I 10, I 11 and I 12; or
if Y is structure I 4 where Y₈ is NH and Y₉ is N(Y₁₁)Y₁₂ where Y₁₁ is H and Y₁₂ is cyclohexyl and X is —(CH₂)ₙ— where n=0, then R can not be naphthyl.

12. The method of claim 10 wherein said at least one inhibitor is substantially pure.

13. The method of claim 10 wherein said excitotoxicity is associated with a condition selected from at least one of stroke, brain injury, brain trauma, hypoxia, ischemia, toxin exposure, tumor growth and excitotoxicity linked to dementia such as in Alzheimer's Disease.

14. A medicament comprising therapeutically effective amounts of at least one endocannabinoid/anandamide transport inhibitor and at least one fatty-acid amide hydrolase inhibitor or physiologically acceptable salts thereof.

15. The medicament of claim 14 wherein said at least one endocannabinoid/anandamide transport inhibitor and said at least one fatty-acid amide hydrolase inhibitor or physiologically acceptable salts thereof are substantially pure.

16. The medicament of claim 14 comprising at least one material selected from an adjuvant, an excipient, a flavoring, a stabilizer and a preservative.

17. The medicament of claim 14 comprising a physiologically acceptable vehicle or carrier.

18. A method of treating excitotoxicity associated with a condition selected from at least one of stroke, brain injury, brain trauma, hypoxia, ischemia, toxin exposure, tumor growth and excitotoxicity linked to dementia such as in Alzheimer's Disease, comprising:
providing a medicament including a therapeutically effective amount of at least one compound that inhibits endocannabinoid/anandamide transport and at least one compound that inhibits FAAH; and
administering said medicament.

19. The method of claim 18 wherein said medicament is administered systemically.

20. The method of claim 18 wherein said medicament is administered locally.
